(19) United States
(12) Patent Application Publication
(10) Pub. No.: US 2007/0036786 A1
(43) Pub. Date: Feb. 15, 2007

(54) Method of treating autoimmune disease using humanized anti-CD16A antibodies

(76) Inventors: Nadine Tuaillon, Gettysburg, PA (US); Leslie S. Johnson, Darnestown, MD (US); Ezio Bonvini, Rockville, MD (US); Kathryn E. Stein, Potomac, MD (US)

Correspondence Address: JONES DAY, 222 EAST 41ST ST, NEW YORK, NY, 10017, US

(21) Appl. No.: 11/485,556

(22) Filed: Jul. 11, 2006

Related U.S. Application Data
(60) Provisional application No. 60/698,623, filed on Jul. 11, 2005.

(51) Int. Cl. A61K 39/395

(52) U.S. Cl. 424/144.1

(57) ABSTRACT

CD16A binding proteins useful for the reduction of a deleterious immune response are described. In one aspect, humanized anti-CD16A antibodies, optionally lacking effector function, are used for treatment of immune disorders such as idiopathic thrombocytopenic purpura and autoimmune hemolytic anemia.

This application claims the benefit of U.S. Provisional Application No. 60/698,623, filed Jul. 11, 2005, which is herein incorporated by reference in its entirety.

### FIELD OF THE INVENTION

The invention relates to CD16A binding proteins and methods for treatment of immune disorders. The invention finds application in the fields of biomedicine and immunology.

### BACKGROUND

Fcγ receptors (FcγR) are cell surface receptors that bind the Fc region of immunoglobulin G (IgG) molecules. Among other functions, these receptors couple the formation of antibody-antigen complexes to effector cell responses. For example, cross-linking of activating Fcγ receptors by immune complexes can result in the phagocytosis of pathogens, killing of foreign and transformed cells by direct cytotoxicity, the clearance of toxic substances, and the initiation of an inflammatory response. Notably, the Fcγ receptors play a key role in autoimmunity. Autoantibody binding to activating Fc receptors triggers the pathogenic sequelae of autoimmune diseases such as idiopathic thrombocytopenic purpura, arthritis, systemic lupus erythrematosus, autoimmune hemolytic anemia, and others.

In humans and rodents there are three classes of Fcγ receptors, designated FcγRI, FcγRII, and FcγRIII (see, Ravetch and Bolland, 2001 Annual Rev. Immunol. 19:275-90; and Ravetch and Kinet, 1991, Annual Rev. Immunol. 9:457-92). FcγRI sites are generally occupied by monomeric IgG, while RII and RIII receptors are generally unoccupied and available to interact with immune complexes. FcγRI, also called CD64, binds monomeric IgG with high affinity, and is present on monocytes and macrophages. FcγRII, also called CD32, binds to multimeric IgG (immune complexes or aggregated IgG) with moderate affinity, and is present on a variety of cell types, including B cells, platelets, neutrophils, macrophages and monocytes. FcγRIII, also called CD16, binds to multimeric IgG with moderate affinity and is the predominant activating FcγR on myeloid cells. FcγRIII is found in two forms. FcγRIIIA (CD16A), a transmembrane signaling form (50-65 kDa), is expressed by NK cells, monocytes, macrophages, and certain T cells. FcγRIIIB (CD16B), a glycosyl-phosphatidyl-inositol anchored form (48 kDa) form, is expressed by human neutrophils. See, e.g., Scallon et al., 1989, Proc. Natl. Acad. Sci. U.S.A. 86:5079-83 and Ravetch et al., 1989, J Exp. Med. 170:481-97. Protein and nucleic acid sequences for CD16A are reported in Genbank as accession numbers P08637 (protein) and X52645 (nucleic acid) and in SWISS-PROT as accession number CAA36870. Protein and nucleic acid sequences for CD16B are reported in Genbank as accession numbers 075015 (protein) and X16863 (nucleic acid) and in SWISS-PROT as CAA34753.

### SUMMARY OF THE INVENTION

In one aspect, the invention provides a CD16A binding protein that may be used for treatment of an individual with an autoimmune disease. CD16A binding proteins of the invention are other than mouse antibodies, and include chimeric, human and humanized anti-CD16A monoclonal antibodies, fragments thereof, single chain antibodies, and other binding proteins comprising a V_{H} domain and/or a V_{L} domain.

In one aspect the CD16A binding protein comprises a Fc region derived from a human IgG heavy chain (e.g., a Fc region derived from human IgG₁) where the Fc region lacks effector function and/or is modified to reduce binding to an Fc receptor. In one embodiment, the CD16A binding protein is not glycosylated, for example, due to a substitution at residue 297 of the Fc region.

In another aspect the CD16A binding protein comprises a Fc region derived from a human IgG heavy chain (e.g., a Fc region derived from human IgG₁) where the Fc region has reduced effector function and/or is modified to reduce binding to an Fc receptor.

In one aspect, the CD16A binding protein is a humanized 3G8 antibody with a V_{H} domain comprising three complementarity determining regions (CDRs) derived from the V_{H} domain of mouse monoclonal antibody (mAb) 3G8. In one embodiment, the V_{H} domain has the sequence of the V_{H} domain of Hu3G8VH-1. In one embodiment, the CDRs of the binding protein have the sequence of the mouse CDRs. In some versions, the V_{H} domain CDRs differ from those of 3G8 at least by one or more of the following substitutions: Val at position 34 in CDR1, Leu at position 50 in CDR2, Phe at position 52 in CDR2, Asn at position 54 in CDR2, Ser at position 60 in CDR2, Ser at position 62 in CDR2, Tyr at position 99 in CDR3, and Asp at position 101 of CDR3. In one embodiment, the V_{H} domain has the sequence of the V_{H} domain of Hu3G8VH-22. In one embodiment V_{H} domain comprises an FR3 domain having the sequence of SEQ ID NO:51. The V_{H} domain may be linked to an antibody heavy chain constant domain, for example the human Cγ1 constant domain.

In some versions the CD16A binding protein has a V_{H} domain having a sequence set forth in Table 4. In some versions the CD16A binding protein has a V_{H} domain that differs from the sequence of Hu3G8VH-1 by one or more of the substitutions shown in Table 1.

In one aspect, the CD16A binding protein is a humanized 3G8 antibody with a V_{L} domain comprising three complementarity determining regions (CDRs) derived from the V_{L} domain of mouse monoclonal antibody 3G8. In one embodiment, the CDRs of the binding protein have the sequence of the mouse CDRs. In some versions, the V_{L} domain CDRs differ from those of 3G8 at least by one or more of the following substitutions: Arg at position 24 in CDR1; Ser at position 25 in CDR1; Tyr at position 32 in CDR1; Leu at position 33 in CDR1, Ala at position 34 in CDR1; Asp, Trp or Ser at position 50 in CDR2; Ala at position 51 in CDR2; Ser at position 53 in CDR2; Ala or Gln at position 55 in CDR2; Thr at position 56 in CDR2; Tyr at position 92 in CDR3, Ser at position 93 in CDR3; and Thr at position 94 in CDR3. In one embodiment, the V_{L} domain has the sequence of the V_{L} domain of Hu3G8VL-1, Hu3G8VL-22 or Hu3G8VL-43. The V_{L} domain may be linked to an antibody light chain constant domain, for example the human C_{K} constant region.

In some versions the CD16A binding protein has a V_{L} domain having a sequence set forth in Table 4. In some versions the CD16A binding protein has a V_{L} domain that differs from the sequence of Hu3G8VL-1 by one or more of the substitutions shown in Table 2.

In one aspect, the CD16A binding protein comprises both a V_{H} domain and a V_{L} domain, as described above (which may be prepared by coexpression of polynucleotides encoding heavy and light chains). Optionally the humanized heavy chain variable region comprises a sequence set forth in Table 4 and/or the humanized light chain variable region comprises a sequence set forth in Table 4. For example, in exemplary embodiments, the binding protein has a heavy chain variable region having the sequence of SEQ ID NO:113 and a light chain variable region having the sequence of SEQ ID NO:96, 100 or 118. In another exemplary embodiment, the binding protein has a heavy chain variable region having the sequence of SEQ ID NO:109 and light chain variable regions having the sequence of SEQ ID NO:96. In another exemplary embodiment, the binding protein has a heavy chain variable region having the sequence of SEQ ID NO:104 and a light chain variable region having the sequence of SEQ ID NO:96.

In an embodiment, the CD16A binding protein is tetrameric antibody comprising two light chains and two heavy chains, said light chains comprising a V_{L} domain and a light chain constant domain and said heavy chains comprising a V_{H} domain and a heavy chain constant domain. In an embodiment, the light chain constant domain is human C_{K} and/or the heavy chain constant region is Cγ1.

In one embodiment of the invention, the CD16A binding protein comprises an antigen binding site that binds CD16A or sCD16A with a binding constant of less than 5 nM.

In one embodiment, the CD16A binding protein comprises an aglycosyl Fc region that has reduced binding to at least one Fc effector ligand compared to a reference CD16A binding protein that comprises an unmodified Fc region (e.g., a human IgG₁ Fc domain glycosylated at position 297). The Fc effector ligand can be FcγRIII or the C1q component of complement.

The invention encompasses a CD16A binding protein, such as a human or humanized anti-CD16A monoclonal antibody, comprising an Fc region that is not glycosylated. As used herein an Fc region which is “not glycosylated” encompasses Fc regions wherein the entire Fc region contains no glycosylation sites, or wherein a specific region within the Fc region is not glycosylated, or wherein a specific residue within the Fc region is not glycosylated. In one specific embodiment, the invention provides a CD16A binding protein comprising an Fc region derived from human IgG₁, where the amino acid corresponding to position 297 of the C_{H}2 domains of the Fc region are aglycosyl (herein referred to a GMA-161). In another embodiment, the invention provides a CD16A binding protein that competes for binding with the GMA-161 and/or binds to the same epitope of CD16A as GMA-161. The present invention also encompasses molecules comprising an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of GMA-161.

The present invention also encompasses antibodies or fragments thereof comprising an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of GMA-161. The present invention further encompasses antibodies or fragments thereof, said antibodies or antibody fragments comprising an amino acid sequence of one or more CDRs that is at least 45%, at least 50%, at least 55%, at least 60%, at least 65%, at least 70%, at least 75%, at least 80%, at least 85%, at least 90%, at least 95%, or at least 99% identical to the amino acid sequence of one or more CDRs of GMA-161. The determination of percent identity of two amino acid sequences can be determined by any method known to one skilled in the art, including BLAST protein searches.

In one embodiment, the invention provides a CD16A binding protein that is a humanized antibody that binds to CD16A and inhibits the binding of Fc receptor to CD16.

In an aspect, the invention provides a pharmaceutical composition comprising of CD16A binding protein described herein and a pharmaceutically acceptable excipient.

In an aspect, the invention provides an isolated polynucleotide, optionally an expression vector, encoding a V_{H} domain of a CD16A binding protein described herein. In an aspect, the invention provides an isolated nucleic acid, optionally an expression vector, encoding a V_{L} domain of a CD16A binding protein described herein. In an aspect, the invention provides a cell, optionally a mammalian cell, comprising a polynucleotide described herein. In an aspect, the invention provides a cell line, optionally a mammalian cell line, expressing a CD16A binding protein described herein.

The invention further provides a method of reducing a deleterious immune response (or undesired immune response) in a mammal comprising administering to a mammal a CD16A binding protein described herein. In an embodiment, reducing the deleterious immune response comprises protecting against antibody-mediated platelet depletion.

In one aspect, the invention provides a method of treating a deleterious immune response in a mammal without inducing neutropenia in the mammal (e.g., severe neutropenia or moderate neutropenia), where the method comprises administering to the mammal a CD16A binding protein having an Fc region derived from human IgG, and where the amino acid at position 297 of the Fc region is aglycosyl.

In embodiments of the above-described methods, the deleterious immune response is an inflammatory response, for example, an inflammatory response caused by an autoimmune disease. In an embodiment, the inflammatory response is caused by idiopathic thrombocytopenic purpura (ITP), rheumatoid arthritis (RA), systemic lupus erythrematosus (SLE), autoimmune hemolytic anemia (AHA), scleroderma, autoantibody triggered urticaria, pemphigus, vasculitic syndromes, systemic vasculitis, Goodpasture's syndrome, multiple sclerosis (MS), psoriatic arthritis, ankylosing spondylitis, Sjögren's syndrome, Reiter's syndrome, Kawasaki's disease, polymyositis and dermatomyositis. Other examples of diseases or conditions that can be treated according to the invention also include any diseases susceptible to treatment with intravenous immunoglobulin (IVIG) therapy (e.g., allergic asthma). The invention provides CD16A binding proteins that both protect against autoimmune diseases and do not result in significant neutrophil diminution in a mammal. In an embodiment, the CD16A binding proteins are anti-CD16A antibodies. These CD16A binding proteins are particularly advantageous for use as human therapeutics. In one aspect, the invention provides a method of treating an autoimmune disease in a mammal without neutrophil diminution or neutropenia in the mammal, by administering a CD16A binding protein having an Fc region derived from human IgG and an aglycosyl amino acid at position 297 of each of the C_{H}2 domains of the Fc region.

In yet another aspect, the invention provides a method of inhibiting the binding of IgG antibodies to FcγRIII on a cell by contacting the cell with a CD16A binding protein under conditions in which the CD16A binding protein binds the FcγRIII on the cell.

In one aspect, the invention provides a method of making a CD16A binding protein with improved therapeutic efficacy in treating a deleterious immune response, comprising the following steps: i) obtaining a first CD16A binding protein, where the first CD16A binding protein comprises an Fc region derived from IgG; and ii) modifying the Fc region of the first CD16A binding protein to produce a second CD16A binding protein that is aglycosylated at position 297 of the Fc region, where the second CD16A binding protein is more effective in treating the deleterious immune response when administered to a mammal than the first CD16A binding protein.

In one aspect, the invention provides a method of making a CD16A binding protein with improved therapeutic efficacy in treating a deleterious immune response, comprising the following steps: i) obtaining a first CD16A binding protein, wherein the first CD16A binding protein comprises an Fc region derived from IgG; and ii) modifying the Fc region of the first CD16A binding protein to produce a second CD16A binding protein that has reduced binding to an Fc effector ligand compared to the unmodified Fc region of the first CD16A binding protein, where the second CD16A binding protein is more effective in treating the deleterious immune response when administered to a mammal than the first CD16A binding protein. In one embodiment, the Fc effector ligand is FcγRIII or the C1q component of complement.

In one aspect the method involves administering a CD16A binding protein to reduce a deleterious immune response in a subject without eliciting one or more significant deleterious effects that result from 3G8 administration, or eliciting significantly lower levels of such effects than does administration of murine 3G8.

In one embodiment of the invention, the improved therapeutic efficacy in treating a deleterious immune response comprises improved effectiveness at protecting against antibody-mediated platelet depletion. The deleterious immune response is optionally due to idiopathic thrombocytopenic purpura (ITP) or the administration of routine monoclonal antibody 6A6 to a muFcγRIII−/−, huFcγRIIIA transgenic mouse.

The invention provides the use of a CD16A binding protein comprising an Fc region derived from a human IgG heavy chain, wherein the Fc region lacks effector function, for treatment of an immune disorder or for preparation of a medicament for treatment of an immune disorder. In other embodiments, the invention provides the use of a CD16A binding protein comprising an Fc region derived from a human IgG heavy chain, wherein the Fc region has reduced effector function, for treatment of an immune disorder or for preparation of a medicament for treatment of an immune disorder.

The use of therapeutic monoclonal antibodies is limited by problems of “first dose” side effects. First dose side effects, range from mild flu-like symptoms to severe toxicity, can be mild to severe, and include symptoms, such as, high fever, chills/rigors, headache, tremor, nausea/vomiting, diarrhea, abdominal pain, malaise, muscle/joint aches and pains, and generalized weakness. The first dose side effects are believed to be caused by lymphokine production and cytokine release stimulated by the Fc region of a mAb binding to and activating an FcγR on an FcγR-containing cell.

The invention thus encompasses CD16A binding proteins that reduced or eliminate at least one symptom associated with first dose side effects by reducing or eliminating binding of the Fc to one or more FcγR. Such CD16A binding proteins comprise a variant Fc region having one or more amino acid modifications, relative to a wild-type Fc region. The modification decreases or eliminates binding of the Fc to one or more FcγRs, relative to a comparable wild-type Fc region. The modification is typically an amino acid substitution. However, the modification can be an amino acid insertion and/or deletion. Typically, the modification occurs in the CH2 and/or hinge region. Alternatively, binding of Fc to one or more FcγRs can be reduced or eliminated by altering or eliminating one or more glycosyl groups on one in more Fc regions. Fc glycosylation can be altered or eliminated by methods well know in the art. For example, Fc glycosylation can be altered by producing the Fc in a cell that is deficient in fucosylation (e.g., fuc6 null cells), or eliminated by deglycosylation enzymes or an amino acid modification that alters or eliminates a glycosylation site (e.g., the N-X-S/T glycosylation site at positions 297-299 in the CH2 domain). FcγR binding can be measured using standard methods known in the art and exemplified herein. The antibodies of the invention are thus particularly useful because they have reduced or no in vivo toxicity caused by lymphokine production or cytokine release.

Methods of measuring lymphokine production and cytokine release are known and routine in the art and encompassed herein. For example, cytokine release may be measured by measuring secretion of cytokines including but not limited to TNF-α, GM-CSF, IFN-γ. See, e.g., U.S. Pat. No. 6,491,916; Isaacs et al., 2001, Rheumatology, 40: 724-738; each of which is incorporated herein by reference in its entirety. Lymphokine production may be measured by measuring secretion of lymphokines including but not limited to Interleukin-2 (IL-2), Interleukin-4 (IL-4), Interleukin-6 (IL-6), Interleukin-12 (IL-12), Interleukin-16 (IL-16), PDGF, TGF-α, TGF-β, TNF-α, TNF-β, GCSF, GM-CSF, MCSF, IFN-α, IFN-β, TFN-γ, IGF-I, IGF-II. For example, see, Isaacs et al., 2001, Rheumatology, 40: 724-738; Soubrane et al., 1993, Blood, 81(1): 15-19; each of which is incorporated herein by reference in its entirety.

As used herein, the term “Fc region” is used to define a C-terminal region of an IgG heavy chain. Although the boundaries may vary slightly, the human IgG heavy chain Fc region is defined to stretch from Cys226 to the carboxy terminus. The Fc region of an IgG comprises two constant domains, CH2 and CH3. The CH2 domain of a human IgG Fc region usually extends from amino acids 231 to amino acid 341. The CH3 domain of a human IgG Fc region usually extends from amino acids 342 to 447. The CH2 domain of a human IgG Fc region (also referred to as “Cγ2” domain) usually extends from amino acid 231-340. The CH2 domain is unique in that it is not closely paired with another domain. Rather, two N-linked branched carbohydrate chains are interposed between the two CH2 domains of an intact native IgG.

Throughout the present specification, the numbering of the residues in an IgG heavy chain is that of the EU index as in Kabat et al., Sequences of Proteins of Immunological Interest, 5^{th} Ed. Public Health Service, NH1, MD (1991), expressly incorporated herein by reference. The “EU index as in Kabat” refers to the numbering of the human IgG1 EU antibody.

The “hinge region” is generally defined as stretching from Glu216 to Pro230 of human IgG1. Hinge regions of other IgG isotypes may be aligned with the IgG1 sequence by placing the first and last cysteine residues forming inter-heavy chain S-S binds in the same positions.

Examples of Fc modifications that will reduce or eliminate at least one symptom associated with first dose side effect include, but are not limited to, those having a substitution at position 233 with proline; or a substitution at position 234 with alanine, or a substitution at position 235 with alanine, or a substitution at position 234 with alanine and at position 235 with an alanine, or a substitution at position 238 with arginine; or a substitution at position 265 with alanine; or a substitution at position 265 with glutamic acid; or a substitution at position 270 with alanine; or a substitution at position 270 with asparagine; or a substitution at position 297 with alanine or glutamine; or a substitution at position 298 with proline or asparagine; or a substitution at position 299 with any amino acid except serine or threonine; or a substitution at position 265 with alanine and at position 297 with alanine; or a substitution at position 265 with alanine and at position 297 with glutamine; or a substitution at position 265 with glutamic acid and at position 297 with alanine; or a substitution at position 265 with glutamic acid and at position 297 with glutamine. The invention further encompasses the combinations of any of the variants listed herein.

The invention encompasses methods for reducing or eliminating at least one symptom associated with first dose side effect in a patient comprising administering an effective amount of one or more antibodies of the invention. The methods of the invention reduce at least one symptom associated with cytokine release syndrome including but not limited to high fever, chills/rigors, headache, tremor, nausea/vomiting, diarrhea, abdominal pain, malaise, muscle/joint aches and pains, and generalized weakness.

### BRIEF DESCRIPTION OF THE FIGURES

FIG. 1 shows results from an ELISA for binding of sCD16A by CD16A binding proteins. Hu3G8-24.43 is an antibody with the heavy chain of Hu3G8VH-24, and the light chain of Hu3G8VL-43. Hu3G8-5.1 is an antibody with the heavy chain of Hu3G8VH-5, and the light chain of Hu3G8VL-1. Ch3G8 is the chimeric 3G8 antibody. Hu1gG1 is an irrelevant immunoglobulin.

FIG. 2 shows results of an assay for binding of humanized and chimeric antibodies to CHO-K1 cells expressing the extracellular domain of CD16A. Hu3G8-22.1 is an antibody with the heavy chain of Hu3G8VH-22, and the light chain of Hu3G8VL-1. Hu3G8-5.1 is an antibody with the heavy chain of Hu3G8VH-5, and the light chain of Hu3G8VL-1. Hu3G8-22.43 is an antibody with the heavy chain of Hu3G8VH-22, and the light chain of Hu3G8VL-43. N297Q indicates the antibody is aglycosylated.

FIG. 3 shows results of a cell based competition assay. The aglycosylated humanized antibodies shown compete with aglycosylated chimeric antibody for binding to CHO-K1 cells expressing the extracellular domain of CD16A.

FIG. 4 shows inhibition of binding of sCD16A to immune complexes. Hu3G8-1.1 is an antibody with the heavy chain Hu3G8VH-1, and the light chain Hu3G8VL-1.

FIG. 5 shows ITP protection in mice injected i.v. with mAb 3G8 (0.5 μg/g) or human IVIG (1 mg/g) one hour before ch6A6 i.p. injection.

FIG. 6 shows ITP protection in mice injected i.v. with mAb 3G8 (0.5 μg/g) or human IVIG (1 mg/g) one hour before ch6A6 i.v. injection.

FIG. 7 shows the absence of ITP protection in mice injected i.v. with ch3G8 (0.5 μg/g) one hour before 6A6 i.p. injection.

FIG. 8 shows protection from ITP in mice injected i.v. with ch3G8 N297Q one hour before ch6A6 i.p. injection.

FIG. 9 shows protection from ITP in mice injected i.v. with ch3G8 N297Q one hour before ch6A6 i.v. injection.

FIG. 10 shows the results of FACS scans of neutrophils following administration of CD16A binding protein or controls. The x-axis shows labeling with antibodies to CD16, and the y-axis shows labeling with antibodies to the Gr-1 antigen. The upper right quadrant shows neutrophils; the upper left quadrant shows other granulocytes and neutrophils that no longer stain with 3G8-FITC.

FIG. 11 shows prevention of AIHA with a humanized anti-CD16A antibody.

FIG. 12 shows inhibition of ch4D5 mediated antibody-dependent cell-mediated cytotoxicity (ADCC) by humanized 3G8 antibodies.

FIGS. 13A-B show inhibition of ch4-4-20 mediated ADCC by mouse 3G8 (FIG. 13A) and humanized 3G8 antibodies (FIG. 13B).

FIG. 14 shows protection of FcγRIII−/−, hCD16A, hCD32A mice against ITP by administration of hu3G8-5.1.

FIGS. 15A-B show protection of FcγRIII−/−, hCD16A mice against ITP by administration of hu3G8-5.1 N297Q. FIG. 15(A) shows data points for each dose at indicated times. FIG. 15(B) shows dose response at the 5 hour time point.

FIGS. 16A-B show the therapeutic effect of administration of aglycosylated humanized antibody subsequent to mice in which thrombocytopenia has been induced. FIG. 16(A) shows administration of Hu3G8-5.1-N297Q. FIG. 16(B) shows administration of Hu3G8-22.1-N297Q and Hu3G8-22.43-N297Q.

FIG. 17 shows the therapeutic effect of a humanized anti-CD16A antibody in treatment of autoimmune hemolytic anemia.

### DETAILED DESCRIPTION

1. Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The practice of the present invention will employ, unless otherwise indicated, conventional techniques of molecular biology (including recombinant techniques), microbiology, cell biology, biochemistry, nucleic acid chemistry, and immunology, which are within the skill of the art. Such techniques are explained fully in the literature, such as, Current Protocols in Immunology (J. E. Coligan et al., eds., 1999, including supplements through 2001); Current Protocols in Molecular Biology (F. M. Ausubel et al., eds., 1987, including supplements through 2001); Molecular Cloning: A Laboratory Manual, third edition (Sambrook and Russet, 2001); PCR: The Polymerase Chain Reaction, (Mullis et al., eds., 1994); The Immunoassay Handbook (D. Wild, ed., Stockton Press NY, 1994); Bioconjugate Techniques (Greg T. Hermanson, ed., Academic Press, 1996); Methods of Immunological Analysis (R. Masseyeff, W. H. Albert, and N. A. Staines, eds., Weinheim: VCH Verlags gesellschaft mbH, 1993); Harlow and Lane Using Antibodies: A Laboratory Manual Cold Spring Harbor Laboratory Press, Cold Spring Harbor, N.Y., 1999; and Beaucage et al. eds., Current Protocols in Nucleic Acid Chemistry John Wiley & Sons, Inc., New York, 2000).

The terms “heavy chain,” “light chain,” “variable region,” “framework region,” “constant domain,” and the like, have their ordinary meaning in the immunology art and refer to domains in naturally occurring immunoglobulins and the corresponding domains of synthetic (e.g., recombinant) binding proteins (e.g., humanized antibodies). The basic structural unit of naturally occurring immunoglobulins (e.g., IgG) is a tetramer having two light chains and two heavy chains. Usually naturally occurring immunoglobulin is expressed as a glycoprotein of about 150,000 daltons, although, as described below, IgG can also be produced in a nonglycosylated form. The amino-terminal (“N”) portion of each chain includes a variable region of about 100 to 110 or more amino acids primarily responsible for antigen recognition. The carboxy-terminal (“C”) portion of each chain defines a constant region, with light chains having a single constant domain and heavy chains usually having three constant domains and a hinge region. Thus, the structure of the light chains of an IgG molecule is N-V_{L}-C_{L}-C and the structure of IgG heavy chains is N-V_{H}-C_{H1}-H-C_{H2}-CH₃-C (where H is the hinge region). The variable regions of an IgG molecule consist of the complementarity determining regions (CDRs), which contain the residues in contact with antigen and non-CDR segments, referred to as framework segments, which maintain the structure and determine the positioning of the CDR loops. Thus, the V_{L} and V_{H} domains have the structure N-FR1, CDR1, FR2, CDR2, FR3, CDR3, FR4-C.

As used herein, the terms “CD16A binding protein,” “CD16A antibody,” and “anti-CD16A antibody,” are used interchangeably and refer to a variety of immunoglobulin-like or immunoglobulin-derived proteins. “CD16A binding proteins” bind CD16A via an interaction with V_{L} and/or V_{H} domains (as distinct from Fc-mediated binding). Examples of CD16A binding proteins include chimeric, humanized and human antibodies (e.g., comprising 2 heavy and 2 light chains), fragments thereof (e.g., Fab, Fab′, F(ab′)₂, and Fv fragments), bifunctional or multifunctional antibodies (see, e.g., Lanzavecchia et al., 1987, Eur. J. Immunol. 17:105), single chain antibodies (see, e.g., Bird et al., 1988, Science 242:423-26), fusion proteins (e.g, phage display fusion proteins), “minibodies” (see, e.g., U.S. Pat. No. 5,837,821) and other antigen binding proteins comprising a V_{L} and/or V_{H} domain or fragment thereof. In one aspect, the CD16A binding protein is a “tetrameric antibody” i.e., having generally the structure of a naturally occurring IgG and comprising both variable and constant domains, (i.e., two light chains comprising a V_{L} domain and a light chain constant domain, such as human C and two heavy chains comprising a V_{H} domain and a heavy chain hinge and constant domains, such as human Cγ1). Except as expressly noted, the mouse antibody 3G8 is specifically excluded from the definition of CD16A binding protein.

When referring to binding proteins or antibodies (as broadly defined herein) the assignment of amino acids to each domain is in accordance with the definitions of Kabat, SEQUENCES OF PROTEINS OF IMMUNOLOGICAL INTEREST (National Institutes of Health, Bethesda, Md., 1987 and 1991). Amino acids from the variable regions of the mature heavy and light chains of immunoglobulins are designated by the position of an amino acid in the chain. Kabat described numerous amino acid sequences for antibodies, identified an amino acid consensus sequence for each subgroup, and assigned a residue number to each amino acid. Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody light chain occupies the equivalent position to an amino acid at position 50 of a mouse antibody light chain. Thus, as used herein in the context of chimeric or humanized antibodies, a reference such as “at position 297 of the Fc region” refers to the amino acid position in an immunoglobulin chain, region of an immunoglobulin chain, or region of a polypeptide derived from an immunoglobulin chain, that corresponds to position 297 of the corresponding human immunoglobulin.

The “Fc region” of immunoglobulins refers to the C-terminal region of an immunoglobulin heavy chain. Although the boundaries of the Fc region may vary somewhat, usually the Fc region is from about position 226-230 extending to the carboxy terminus of the polypeptide (and encompassing the C_{H}2 and C_{H}3 domains). Sequences of human Fc regions are found in Kabat, supra. In addition, a variety of allotypic variants are known to exist.

An “Fc effector ligand” is a ligand that binds to the Fe region of an IgG antibody, thereby activating effector mechanisms resulting in the clearance and destruction of pathogens. Fc effector ligands include three cellular Fc receptors types—FcRγI, FcRγII, and FcRγIII. The multiple isoforms of each of the three Fc receptor types are also included. Accordingly, the term “Fc effector ligand” includes both FcγRIIIA (CD16A) and FcγRIIIB (CD16B). The term “Fc effector ligand” also includes the neonatal Fc receptor (Fcγn) and the C1q component of complement. Binding of IgG to the Fc receptors triggers a variety of biological processes including antibody-dependent cell-mediated cytotoxicity (ADCC), release of inflammatory mediators, control of antibody production, clearance of immune complexes and destruction of antibody-coated particles. Binding of the C1q component of complement to IgG activates the complement system. Activation of complement plays important roles in opsonization, lysis of cell pathogens, and inflammatory responses.

“Effector function” as used herein refers to a biochemical event that results from the interaction of an antibody Fc region with an Fc receptor or ligand. Effector functions include but are not limited to antibody-dependent cell-mediated cytotoxicity (ADCC), antibody dependent cell mediated phagocytosis (ADCP), and complement-dependent cytotoxicity (CDC). Effector functions include both those that operate after the binding of an antigen and those that operate independent of antigen binding.

As used herein, an Fc region that “lacks effector function” does not bind the Fc receptor and/or does not bind the C1q component of complement and trigger the biological responses characteristic of such binding. As used herein an Fc region that has “reduced effector function” has reduced affinity for an Fc receptor and/or has reduced affinity for the C1q component of complement and thus triggers the biological responses characteristic of such binding less effectively. Molecules comprising Fc regions with reduced effector function may have reduced effector function activity by at least 10%, at least 20%, at least 50%, at least 80%, at least 80%, at least 90%, or at least 99% compared to a molecule comprising a wild-type Fc region have wild-type effector function activity.

The term “glycosylation site” refers to an amino acid residue that is recognized by a mammalian cell as a location for the attachment of sugar residues. Amino acid residues to which carbohydrates, such as oligosaccharides, are attached are usually asparagine (N-linkage), serine (O-linkage), and threonine (O-linkage) residues. The specific sites of attachment usually have a characteristic sequence of amino acids, referred to as a “glycosylation site sequence.” The glycosylation site sequence for N-linked glycosylation is: -Asn-X-Ser- or -Asn-X-Thr-, where X can be any of the conventional amino acids, other than proline. The Fc region of human IgG has two glycosylation sites, one in each of the C_{H}2 domains. The glycosylation that occurs at the glycosylation site in the C_{H}2 domain of human IgG is N-linked glycosylation at the asparagine at position 297 (Asn 297).

The term “chimeric,” when referring to antibodies, has the ordinary meaning in the art and refers to an antibody in which a portion of a heavy and/or light chain is identical to or homologous with an antibody from one species (e.g., mouse) while the remaining portion is identical to or homologous with an antibody of another species (e.g., human).

As used herein, the term “humanized” has its usual meaning in the art. In general terms, humanization of a non-human antibody involves substituting the CDR sequences from non-human immunoglobulin V_{L} and V_{H} regions into human framework regions. Further, as used herein, “humanized” antibodies may comprise additional substitutions and mutations in the CDR and/or framework regions introduced to increase affinity or for other purposes. For example, substitution of nonhuman framework residues in the human sequence can increase affinity. See, e.g., Jones et al., 1986, Nature 321:522-25; Queen et al., 1989, Proc. Natl. Acad. Sci. U.S.A. 86:10029-33; Foote and Winter, 1992, J Mol. Biol. 224:487-99; Chothia et al., 1989, Nature 342:877-83; Riechmann et al., 1988, Nature 332:323-27; Co et al., 1991, Proc. Natl. Acad. Sci. U.S.A. 88:2869-73; Padlan, 1991, Mol. Immunol. 28:489-98. The resulting variable domains have non-human CDR sequences and framework sequences derived from human antibody framework sequence(s) or a human consensus sequence (e.g., as disclosed in Kabat, supra). A variety of different human framework regions may be used singly or in combination as a basis for the humanized monoclonal antibodies of the present invention. The framework sequences of a humanized antibody are “substantially human,” by which is meant that at least about 70% of the human antibody sequence, usually at least about 80% human, and most often at least about 90% of the framework sequence is from human antibody sequence. In some embodiments, the substantially human framework comprises a serine at position 113 of the V_{H} FR4 domain (e.g., SEQ ID NO:64). As used herein, a “humanized antibody” includes, in addition to tetrameric antibodies, single chain antibodies, antibody fragments and the like that comprise CDRs derived from a nonhuman antibody and framework sequences derived from human framework regions.

As used herein, “mammals” include humans, non-human primates, rodents, such as, mice and rats, and other mammals.

As used herein, “neutropenia” has its ordinary meaning, and refers to a state in which the number of neutrophils circulating in the blood is abnormally low. The normal level of neutrophils in human blood varies slightly by age and race. The average adult level is about 1500 cells/mm³ of blood. Neutrophil counts less than 500 cells/mm³ result in great risk of severe infection. Generally, in humans, severe neutropenia is defined by a blood neutrophil count less than about 500 cells/mm³, and moderate neutropenia is characterized by a blood neutrophil count from about 500-1000 cells/mm³.

As used herein, “treatment” refers to clinical intervention in an attempt to alter the disease course of the individual or cell being treated, and can be performed either for prophylaxis or during the course of clinical pathology. Therapeutic effects of treatment include without limitation, preventing occurrence or recurrence of disease, alleviation of symptoms, diminishment of any direct or indirect pathological consequences of the disease, decreasing the rate of disease progression, amelioration or palliation of the disease state, and remission or improved prognosis.

An “effective amount” is an amount sufficient to effect a beneficial or desired clinical result upon treatment. An effective amount can be administered to a patient in one or more doses. A “therapeutically effective amount” is an amount that is sufficient to palliate, ameliorate, stabilize, reverse or slow the progression of the disease, or otherwise reduce the pathological consequences of the disease, or reduce the symptoms of the disease. The amelioration or reduction need not be, and usually is not, permanent, but may be for a period of time ranging from at least one hour, at least one day, or at least on week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition and the form and effective concentration of the binding protein administered. An “inflammation reducing amount” is an amount that reduces inflammation in a subject. A reduction in inflammation can be assessed by art known criteria, including decreased C-reactive protein levels, decreased consumption of complement, reduced immune complex deposition at sites of inflammation (e.g., joints in subjects with RA, kidney in subjects with lupus, myelin sheath, etc.), reduced cytokine release, migration of macrophages and neutrophils, and the like.

“Substantial sequence identity,” as used herein, refers to two or more sequences or subsequences (e.g., domains) that have at least about 80% amino acid residue identity, preferably at least about 90%, or at least about 95% identity when compared and aligned for maximum correspondence. Sequence identity between two similar sequences (e.g., antibody variable regions) can be measured by (1) aligning the sequences to maximize the total number of identities across the entire length of the sequences, or across the entire length of the shorter of the two sequences, if of different lengths (and where the length of the aligned sequences or shorter of the aligned sequences is “L” residues); (2) counting the number of positions (not including the number “E” residues designated as excluded from the comparison) at which there is an amino acid identity, where the number of identities is designated “N”; (3) and dividing the N by the “L” minus “E.” For example, in a comparison of two sequences each of length 80 residues, in which 6 specific residues are excluded from the comparison and for which there are 65 identities in the remaining 74 positions, the sequence identity would be N/(L−E) or 65/(80−6) or 87.8%. (Residues might be specified as “excluded” from the calculation when, for illustration but not limitation, they are in a non-antibody domain of fusion protein.) Alternatively, optimal alignment and sequence identity can be calculated by computerized implementations of algorithms described in Smith & Waterman, 1981, Adv. Appl. Math. 2:482 [local homology algorithm], Needleman & Wunsch, 1970, J. Mol. Biol. 48:443 [homology alignment algorithm], Pearson & Lipman, 1988, Proc. Natl. Acad. Sci. USA 85:2444, [search for similarity method], or Altschul et al., 1990, J. Mol. Biol. 215:403-10 [BLAST algorithm]. See Ausubel et al., supra and GAP, BESTFIT, FASTA, and TFASTA in the Wisconsin Genetics Software Package, Genetics Computer Group, 575 Science Dr., Madison, Wis.). When using any of the aforementioned algorithms, the default parameters (for Window length, gap penalty, etc.) are used. An amino acid or nucleic acid sequence is “substantially similar to” a second sequence when the degree of sequence identity is at least about 70% identical, preferably at least about 80%, or at least about 90%, or even at least about 95%, identical. Sequences that are substantially identical are also substantially similar.

As used herein, a polypeptide, polypeptide domain or region, or amino acid sequence is “derived from” another when the two sequences are identical or substantially similar and have a similar biological function. For example, in a humanized mouse monoclonal antibody the complementary determining regions (CDRs) are “derived from” the corresponding CDRs of the mouse monoclonal antibody, and the variable domain framework regions can be “derived from” framework sequences of the corresponding human antibody. It will be apparent that one domain, etc., can be derived from a parental domain, etc., even though the two differ in sequence due to, for example, the introduction of mutations that affect, or alternatively do not change, binding affinity or other properties of the protein in which the domain, etc., is contained, such as those described herein. It will also be understood that normally a domain, etc., “derived from” a parental domain, etc., is made, produced or designed using materials (e.g. genetic material) or information (e.g., nucleotide or amino acid sequence) from the parental molecule.

Standard abbreviations are used for amino acids: alanine, Ala (A); serine, Ser (S); threonine, Thr (T); aspartic acid, Asp (D); glutamic acid, Glu (E); asparagine, Asn (N); glutamine, Gln (Q); arginine, Arg (R); lysine, Lys (K); isoleucine, Ile (I); leucine, Leu (L); methionine, Met (M); valine, Val (V); phenylalanine, Phe (F); tyrosine, Tyr (Y); tryptophan, Trp (W); glycine, Gly (G); histidine, His (H); proline, Pro (P); and cysteine, Cys (C).

As used herein, “GMA-161” refers to a CD16A binding protein wherein the V_{L} chain has the sequence as forth in SEQ ID NO: 98 and the V_{H} chain has the sequence as set forth in SEQ ID NO: 120.

2. Introduction

The FcγRIIIA receptor, CD16A, plays a role in coupling cytotoxic and immune complex antibodies to effector responses. It is believed that the interaction of the FcγRIIIA receptor and immunoglobulin aggregates (e.g. immune complexes) present in autoimmune diseases and other pathogenic conditions results in a deleterious inflammatory response in subjects. Without intending to be bound by a specific mechanism, it is believed that reducing the interaction of the FcγRIIIA receptor (generally referred to herein as “CD16A” or “the CD16A receptor”) and immunoglobulin aggregates will alleviate this inflammatory response. Also without intending to be bound by a specific mechanism, it is believed that one method for reducing the interaction of CD16A and immunoglobulin aggregates is by use of anti-CD16A antibodies, or other CD16A binding proteins, to block the interaction.

Monoclonal antibody 3G8 (“mAb 3G8”) is a mouse monoclonal antibody that binds the Fc-binding domain of human CD16A and B with a Kₐ of 1×10⁹ M⁻¹ (Fleit et al., 1982, Proc. Natl. Acad. Sci. U.S.A. 79:3275-79). 3G8 blocks the binding of human IgG₁ immune complexes to isolated human NK cells, monocytes and neutrophils, as well as to CD16A-transfected 293 cells. Experiments in which mAb 3G8 has been administered to human patients for treatment of idiopathic thrombocytopenic purpura (ITP) have been conducted (Clarkson et al., 1986, N. Engl. J Med. 314:1236-39; Soubrane, et al., 1993, Blood 81:15-19). Administration of the 3G8 antibody was reported to result in increased platelet levels and was accompanied by one or more significant side effects, including a HAMA response, cytokine release syndrome, and/or pronounced neutropenia.

The present invention provides novel CD16A binding proteins, including humanized and/or aglycosylated monoclonal antibodies, and methods for reducing a deleterious immune response in a subject by administering the proteins. Administration of these binding proteins is shown to be protective in well established models for two distinct autoimmune diseases: autoimmune hemolytic anemia (AHA) and idiopathic thrombocytopenic purpura. These results are indicative of efficacy of this treatment for other autoimmune diseases as well. Moreover, the inventors have discovered that, unexpectedly, administration of anti-CD16A antibodies with altered effector function (e.g., aglycosylated antibodies) protects against the deleterious immune responses characteristic of autoimmune disorders without inducing acute severe neutropenia. Thus, the invention provides new reagents and methods for antibody-mediated effected treatment of autoimmune conditions without pronounced side-effects observed using alternative treatments.

3. CD16A Binding Proteins

A variety of CD16A binding proteins may be used in the methods of the invention. Suitable CD16A binding proteins include human or humanized monoclonal antibodies as well as CD16A binding antibody fragments (e.g., scFv or single chain antibodies, Fab fragments, minibodies) and another antibody-like proteins that bind to CD16A via an interaction with a light chain variable region domain, a heavy chain variable region domain, or both.

In some embodiments, the CD16A binding protein for use according to the invention comprises a V_{L} and/or V_{H} domain that has one or more CDRs with sequences derived from a non-human anti-CD16A antibody, such as a mouse anti-CD16A antibody, and one or more framework regions derived from framework sequences of one or more human immunoglobulins. A number of non-human anti-CD16A monoclonal antibodies, from which CDR and other sequences may be obtained, are known (see, e.g., Tamm and Schmidt, 1996, J. Immunol. 157:1576-81; Fleit et al., 1982, Proc. Natl. Acad. Sci. U.S.A. 79:3275-79; LEUKOCYTE TYPING II: HUMAN MYELOID AND HEMATOPOIETIC CELLS, Reinherz et al., eds. New York: Springer-Verlag; 1986; LEUCOCYTE TYPING III: WHITE CELL DIFFERENTIATION ANTIGENS McMichael A J, ed., Oxford: Oxford University Press, 1986); LEUKOCYTE TYPING IV: WHITE CELL DIFFERENTIATION ANTIGENS, Kapp et al., eds. Oxford Univ. Press, Oxford; LEUKOCYTE TYPING V: WHITE CELL DIFFERENTIATION ANTIGENS, Schlossman et al., eds. Oxford Univ. Press, Oxford; LEUKOCYTE TYPING VI: WHITE CELL DIFFERENTIATION ANTIGENS, Kishimoto, ed. Taylor & Francis. In addition, as shown in the Examples, new CD16A binding proteins that recognize human CD16A expressed on cells can be obtained using well known methods for production and selection of monoclonal antibodies or related binding proteins (e.g., hybridoma technology, phage display, and the like). See, for example, O'Connel et al., 2002, J. Mol. Biol. 321:49-56; Hoogenboom and Chames, 2000, Imm. Today 21:371078; Krebs et al., 2001, J. Imm. Methods 254:67-84; and other references cited herein. Monoclonal antibodies from a non-human species can be chimerized or humanized using techniques of antibody humanization known in the art.

Alternatively, fully human antibodies against CD16A can be produced using transgenic animals having elements of a human immune system (see, e.g., U.S. Pat. Nos. 5,569,825 and 5,545,806), using human peripheral blood cells (Casali et al., 1986, Science 234:476), by screening a DNA library from human B cells according to the general protocol outlined by Huse et al., 1989, Science 246:1275, and by other methods.

It is contemplated that, for some purposes, it may be advantageous to use CD16A binding proteins that bind the CD16A receptor at the same epitope bound by 3G8, or at least sufficiently close to this epitope to block binding by 3G8. Methods for epitope mapping and competitive binding experiments to identify binding proteins with the desired binding properties are well known to those skilled in the art of experimental immunology. See, for example, Harlow and Lane, cited supra; Stahl et al., 1983, Methods in Enzymology 9:242-53; Kirkland et al., 1986, J. Immunol. 137:3614-19; Morel et al., 1988, Molec. Immunol. 25:7-15; Cheung et al., 1990, Virology 176:546-52; and Moldenhauer et al., 1990, Scand. J. Immunol. 32:77-82. Also see Examples and §3G(i), infra. For instance, it is possible to determine if two antibodies bind to the same site by using one of the antibodies to capture the antigen on an ELISA plate and then measuring the ability of the second antibody to bind to the captured antigen. Epitope comparison can also be achieved by labeling a first antibody, directly or indirectly, with an enzyme, radionuclide or fluorophore, and measuring the ability of an unlabeled second antibody to inhibit the binding of the first antibody to the antigen on cells, in solution, or on a solid phase.

It is also possible to measure the ability of antibodies to block the binding of the CD16A receptor to immune complexes formed on ELISA plates. Such immune complexes are formed by first coating the plate with an antigen such as fluorescein, then applying a specific anti-fluorescein antibody to the plate. This immune complex then serves as the ligand for soluble Fc receptors such as sFcγRIIIa. Alternatively a soluble immune complex may be formed and labeled, directly or indirectly, with an enzyme radionuclide or fluorophore. The ability of antibodies to inhibit the binding of these labeled immune complexes to Fc receptors on cells, in solution or on a solid phase can then be measured.

CD16A binding proteins of the invention may or may not comprise a human immunoglobulin Fc region. Fc regions are not present, for example, in scFv binding proteins. Fc regions are present, for example, in human or humanized tetrameric monoclonal IgG antibodies. As described in detail below, in some embodiments of the present invention, the CD16A binding protein includes an Fc region that has an altered effector function, e.g., reduced affinity for an effector ligand such as an Fc receptor or CI component of complement compared to the unaltered Fc region (e.g., Fc of naturally occurring IgG₁ proteins). In one embodiment the Fc region is not glycosylated at the Fc region amino acid corresponding to position 297. Such antibodies lack Fc effector function.

Thus, in some embodiments of the invention, the CD16A binding protein does not exhibit Fc-mediated binding to an effector ligand such as an Fc receptor or the C1 component of complement due to the absence of the Fc domain in the binding protein while, in other cases, the lack of binding or effector function is due to an alteration in the constant region of the antibody.

The invention encompasses CD16A binding proteins, comprising a variant Fc region, having one or more amino acid modifications (e.g., substitutions, but also including insertions or deletions) in one or more regions, which modifications alter, e.g., increase or decrease, the affinity of the variant Fc region for an FcγR. Examples of such variant Fc regions are disclosed in U.S. application Ser. No. 10/754,922, filed on Jan. 9, 2004 and Ser. No. 10/902,588, filed on Jul. 28, 2004 each of which is incorporated herein by reference in its entirety. In some embodiments, the invention encompasses CD16A binding proteins comprising a variant Fc region, wherein said variant Fc region comprises at least one amino acid modification relative to a wild-type Fc region, which variant Fc region does not bind any FcγR or binds with a reduced affinity, relative to a comparable molecule comprising the wild-type Fc region, as determined by standard assays (e.g., in vitro assays) known to one skilled in the art. Examples of such variants include but are not limited to a substitution at position 233 with proline; or a substitution at position 238 with arginine; or a substitution at position 265 with alanine; or a substitution at position 265 with glutamic acid; or a substitution at position 270 with alanine; or a substitution at position 270 with asparagine; or a substitution at position 297 with alanine or glutamine; or a substitution at position 298 with proline or asparagine; or a substitution at position 299 with any amino acid except serine or threonine; or a substitution at position 234 with alanine; or a substitution at position 235 with alanine; or a substitution at position 234 with alanine and at position 235 with alanine; or a substitution at position 265 with alanine and at position 297 with alanine; or a substitution at position 265 with alanine and at position 297 with glutamine; or a substitution at position 265 with glutamic acid and at position 297 with alanine; or a substitution at position 265 with glutamic acid and at position 297 with glutamine. The invention further encompasses the combinations of any of the variants listed herein.

Preferably, said one or more amino acid modification increases the affinity of the variant Fc region for FcγRIIIA and/or FcγRIIA. In a preferred embodiment, the molecules of the invention further specifically bind FcγRIIB (via the Fc region) with a lower affinity than a comparable molecule (i.e., having the same amino acid sequence as the molecule of the invention except for the one or more amino acid modifications in the Fc region) comprising the wild-type Fc region binds FcγRIIB. In some embodiments, the invention encompasses molecules with variant Fc regions, having one or more amino acid modifications, which modifications increase the affinity of the variant Fc region for FcγRIIIA and/or FcγRIIA and enhance the affinity of the variant Fc region for FcγRIIB relative to a comparable molecule with a wild-type Fc region. In other embodiments, the invention encompasses molecules with variant Fc regions, having one or more amino acid modifications, which modifications increase the affinity of the variant Fc region for FcγRIIIA and/or FcγRIIA but do not alter the affinity of the variant Fc regions for FcγRIIB relative to a comparable molecule with a wild-type Fc region.

4. CD16A Binding Proteins Comprising CDR Sequences Similar to mAb 3G8 CDR Sequences

CD16A binding proteins that can be used in the practice of the invention include proteins comprising a CDR sequence derived from (i.e., having a sequence the same as or similar to) the CDRs of the mouse monoclonal antibody 3G8. Complementary cDNAs encoding the heavy chain and light chain variable regions of the mouse 3G8 monoclonal antibody, including the CDR encoding sequences, were cloned and sequenced as described in the Examples. The nucleic acid and protein sequences of 3G8 are provided below and are designated SEQ ID NOs:1 and 2 (V_{L}) and SEQ ID NOs:3 and 4 (V_{H}). Using the mouse variable region and CDR sequences, a large number of chimeric and humanized monoclonal antibodies, comprising complementary determining regions derived from 3G8 CDRs were produced and their properties analyzed. To identify humanized antibodies that bind CD16A with high affinity and have other desirable properties, antibody heavy chains comprising a V_{H} region with CDRs derived from 3G8 were produced and combined (by coexpression) with antibody light chains comprising a V_{L} region with CDRs derived from 3G8 to produce a tetrameric antibody for analysis. Properties of the resulting tetrameric antibodies were determined as described below. As described below, CD16A binding proteins comprising 3G8 CDRs, such as the humanized antibody proteins described herein below, may be used according to the invention to reduce a deleterious immune response.

A. V_{H} Region

In one aspect, the CD16A binding protein of the invention may comprise a heavy chain variable domain in which at least one CDR (and usually three CDRs) have the sequence of a CDR (and more typically all three CDRs) of the mouse monoclonal antibody 3G8 heavy chain and for which the remaining portions of the binding protein are substantially human (derived from and substantially similar to, the heavy chain variable region of a human antibody or antibodies).

In an aspect, the invention provides a humanized 3G8 antibody or antibody fragment containing CDRs derived from the 3G8 antibody in a substantially human framework, but in which at least one of the CDRs of the heavy chain variable domain differs in sequence from the corresponding mouse antibody 3G8 heavy chain CDR. For example, in one embodiment, the CDR(s) differs from the 3G8 CDR sequence at least by having one or more CDR substitutions shown in Table 1 (e.g., valine at position 34 in CDR1, leucine at position 50 in CDR2, phenylalanine at position 52 in CDR2, tyrosine at position 52 in CDR2, aspartic acid at position 52 in CDR2, asparagine at position 54 in CDR2, serine at position 60 in CDR2, serine at position 62 in CDR2, tyrosine at position 99 in CDR3, and/or aspartic acid at position 101 of CDR3). Suitable CD16A binding proteins may comprise 0, 1, 2, 3, or 4, or more of these substitutions (and often have from 1 to 4 of these substitutions) and optionally can have additional substitutions as well.

In one embodiment, a CD16A binding protein may comprise a heavy chain variable domain sequence that is the same as, or similar to, the V_{H} domain of the Hu3G8VH-1 construct, the sequence of which is provided in Table 4. For example, the invention provides a CD16A binding protein comprising a V_{H} domain with a sequence that (1) differs from the V_{H} domain of Hu3G8VH-1 by zero, one, or more than one of the CDR substitutions set forth in Table 1; (2) differs from the V_{H} domain of Hu3G8VH-1 by zero, one or more than one of the framework substitutions set forth in Table 1; and (3) is at least about 80% identical, often at least about 90%, and sometimes at least about 95% identical, or even at least about 98% identical to the Hu3G8VH-1 V_{H} sequence at the remaining positions.

Exemplary V_{H} domains of CD16A binding proteins of the invention have the sequence of Hu3G8VH-5 and Hu3G8VH-22, as shown in Tables 3 and 6.

The V_{H} domain may have a sequence that differs from that of Hu3G8VH-1 (Table 4) by at least one, at least two, at least three, at least four, at least five, or at least six of the substitutions shown in Table 1. These substitutions are believed to result in increased affinity for CD16A and/or reduce the immunogenicity of a CD16A binding protein when administered to humans. In certain embodiments, the degree of sequence identity with the Hu3G8VH-1 V_{H} domain at the remaining positions is at least about 80%, at least about 90%, at least about 95% or at least about 98%.

TABLE 1
V_{H} Domain Substitutions

| | Kabat | | |
|---|---|---|---|
| No. | Position | Region | Substitutions |
| 1 | 2 | FR1 | Ile |
| 2 | 5 | FR1 | Lys |
| 3 | 10 | FR1 | Thr |
| 4 | 30 | FR1 | Arg |
| 5 | 34 | CDR1 | Val |
| 6 | 50 | CDR2 | Leu |
| 7 | 52 | CDR2 | Phe or |
| | | | Tyr or |
| | | | Asp |
| 8 | 54 | CDR2 | Asn |
| 9 | 60 | CDR2 | Ser |
| 10 | 62 | CDR2 | Ser |
| 11 | 70 | FR3 | Thr |
| 12 | 94 | FR3 | Gln or |
| | | | Lys or |
| | | | Ala or |
| | | | His |
| 13 | 99 | CDR3 | Tyr |
| 14 | 101 | CDR3 | Asp |

For illustration and not limitation, the sequences of a number of CD16A binding protein VH domains are shown in Table 4. As described in the Examples, infra, heavy chains comprising these sequences fused to a human Cγ1 constant region were coexpressed with the hu3G8VL-1 light chain (described below) to form tetrameric antibodies, and binding of the antibodies to CD16A was measured to assess the effect of amino acid substitutions compared to the hu3G8VH-1 V_{H} domain. Constructs in which the VH domain has a sequence of hu3G8VH-1, 2, 3, 4, 5, 8, 12, 14, 16, 17, 18, 19, 20, 22, 23, 24, 25, 26, 27, 28, 29, 30, 31, 32, 33, 34, 35, 36, 37, 42, 43, 44, and 45 showed high affinity binding, with hu3G8VH-6 and -40 VH domains showing intermediate binding. CD16A binding proteins comprising the V_{H} domains of hu3G8VH-5 and hu3G8VH-22 are considered to have particularly favorable binding properties.

B. V_{L} Region

Similar studies were conducted to identify light chain variable domain sequences with favorable binding properties. In one aspect, the invention provides a CD16A binding protein containing a light chain variable domain in which at least one CDR (and usually three CDRs) has the sequence of a CDR (and more typically all three CDRs) of the mouse monoclonal antibody 3G8 light chain and for which the remaining portions of the binding protein are substantially human (derived from and substantially similar to, the heavy chain variable region of a human antibody or antibodies).

In one aspect, the invention provides a humanized 3G8 antibody or antibody fragment containing CDRs derived from the 3G8 antibody in a substantially human framework, but in which at least one of the CDRs of the light chain variable domain differs in sequence from the mouse monoclonal antibody 3G8 light chain CDR. In one embodiment, the CDR(s) differs from the 3G8 sequence at least by having one or more amino acid substitutions in a CDR, such as, one or more substitutions shown in Table 2 (e.g., arginine at position 24 in CDR1; serine at position 25 in CDR1; tyrosine at position 32 in CDR1; leucine at position 33 in CDR1; aspartic acid, tryptophan or serine at position 50 in CDR2; serine at position 53 in CDR2; alanine or glutamine at position 55 in CDR2; threonine at position 56 in CDR2; serine at position 93 in CDR3; and/or threonine at position 94 in CDR3). In various embodiments, the variable domain can have 0, 1, 2, 3, 4, 5, or more of these substitutions (and often have from 1 to 4 of these substitutions) and optionally, can have additional substitutions as well.

In one embodiment, a suitable CD16A binding protein may comprise a light chain variable domain sequence that is the same as, or similar to, the V_{L} domain of the Hu3G8VL-1 construct, the sequence of which is provided in Table 5. For example, the invention provides a CD16A binding protein comprising a V_{L} domain with a sequence that (1) differs from the V_{L} domain of Hu3G8VL-1 by zero, one, or more of the CDR substitutions set forth in Table 2, (2) differs from the V_{L} domain of Hu3G8VL-1 by zero, one or more of the framework substitutions set forth in Table 2; and (3) is at least about 80% identical, often at least about 90%, and sometimes at least about 95% identical, or even at least about 98% identical to the Hu3G8VL-1 V_{L} sequence at the remaining positions.

Exemplary V_{L} domains of CD16A binding proteins of the invention have the sequence of Hu3G8VL-I or Hu3G8VL-43, as shown in Tables 4 and 6.

The V_{L} domain may have a sequence that differs from that of Hu3G8VL-1 (Table 5) by zero, one, at least two, at least 3, at least 4, at least 5, at least 6, at least 7, at least 8, or at least 9 of the substitutions shown in Table 2. These substitutions are believed to result in increased affinity for CD16A and/or reduce the immunogenicity of a CD16A binding protein when administered to humans. In certain embodiments, the degree of sequence identity at the remaining positions is at least about 80%, at least about 90%, at least about 95% or at least about 98%.

TABLE 2
V_{L} Domain Substitutions

| | Kabat | | |
|---|---|---|---|
| No. | Position | Region | Substitutions |
| 1 | 24 | CDR1 | Arg |
| 2 | 25 | CDR1 | Ser |
| 3 | 32 | CDR1 | Tyr |
| 4 | 33 | CDR1 | Leu |
| 5 | 50 | CDR2 | Asp or |
| | | | Trp or |
| | | | Ser |
| 6 | 51 | CDR2 | Ala |
| 7 | 53 | CDR2 | Ser |
| 8 | 55 | CDR2 | Ala or |
| | | | Gln |
| 9 | 56 | CDR2 | Thr |
| 10 | 93 | CDR3 | Ser |
| 11 | 94 | CDR3 | Thr |

For illustration and not limitation, the sequences of a number of CD16A binding protein V_{L} domains are shown in Table 5. As described in the Examples, infra, light chains comprising these sequences fused to a human CK constant domain were coexpressed with the Hu3GVH-1 heavy chain (described above) to form tetrameric antibodies, and the binding of the antibodies to CD16A was measured to assess the effect of amino acid substitutions compared to the Hu3G8VL-1 V_{L} domain. Constructs in which the V_{L} domain has a sequence of hu3G8VL-1, 2, 3, 4, 5, 10, 16, 18, 19, 21, 22, 24, 27, 28, 32, 33, 34, 35, 36, 37, and 42 showed high affinity binding and hu3G8VL-15, 17, 20, 23, 25, 26, 29, 30, 31, 38, 39, 40 and 41 showed intermediate binding. CD16A binding proteins comprising the V_{L} domains of hu3G8VL-1, hu3G8VL-22, and hu3G8VL-43 are considered to have particularly favorable binding properties.

C. Combinations of V_{L} and/or V_{H} Domains

As is known in the art and described elsewhere herein, immunoglobulin light and heavy chains can be recombinantly expressed under conditions in which they associate to produce a tetrameric antibody, or can be so combined in vitro. Similarly, combinations of V_{L} and/or V_{H} domains can be expressed in the form of single chain antibodies, and still other CD16A binding proteins that comprise a V_{L} and/or V_{H} domain can be expressed by known methods. It will thus be appreciated that a 3G8-derived V_{L}-domain described herein can be combined with a 3G8-derived V_{H}-domain described herein to produce a CD16A binding protein, and all such combinations are contemplated.

For illustration and not for limitation, examples of useful CD16A binding proteins are those comprising at least one V_{H} domain and at least one V_{L} domain, where the V_{H} domain is from hu3G8VH-1, hu3G8VH-22 or hu3G8VH-5 and the V_{L} domain is from hu3G8VL-1, hu3G8VL-22 or hu3G8VL-43. In particular, humanized antibodies that comprise hu3G8VH-22 and either hu3G8VL-1, hu3G8VL-22 or hu3G8VL-43, or hu3G8VH-5 and hu3G8VL-1 have favorable properties.

It will be appreciated by those of skill that the sequences of V_{L} and V_{H} domains described here can be further modified by art-known methods such as affinity maturation (see Schier et al., 1996, J. Mol. Biol. 263:551-67; Daugherty et al., 1998, Protein Eng. 11:825-32; Boder et al., 1997, Nat. Biotechnol. 15:553-57; Boder et al., 2000, Proc. Natl. Acad. Sci. U.S.A. 97:10701-705; Hudson and Souriau, 2003, Nature Medicine 9:129-39). For example, the CD16A binding proteins can be modified using affinity maturation techniques to identify proteins with increased affinity for CD16A and/or decreased affinity for CD16B.

D. Constant Domains and Fc Region

As noted above, the CD16A binding protein of the invention may contain light chain and/or heavy chain constant regions (including the hinge region connecting the C_{H1} and C_{H2} domains in IgG molecules). It is contemplated that a constant domain from any type (e.g., IgM, IgG, IgD, IgA and IgE) of immunoglobulin may be used. The constant domain for the light chain can be lambda or kappa. The constant domain for the heavy chain can be any isotype (e.g., IgG₁ IgG₂, IgG₃ and IgG₄). Chimeric constant domains, portions of constant domains, and variants of naturally occurring human antibody constant domains (containing deletions, insertions or substitutions of amino acid residues) may be used. For instance, a change in the amino acid sequence of a constant domain can be modified to provide additional or different properties, such as altered immunogenicity or half-life of the resultant polypeptide. The changes range from insertion, deletion or substitution of a small number (e.g., less than ten, e.g., one, two, three or more) amino acid residues to substantial modifications of the constant region domain. Changes contemplated include those that affect the interaction with membrane receptors, complement fixation, persistence in circulation, and other effector functions. For example, the hinge or other regions can be modified as described in U.S. Pat. No. 6,277,375. In particular, it will often be advantageous to delete or alter amino acids of the Fc region. For example, the Fc region can be modified to reduce or eliminate binding to Fc effector ligands such as FcγRIII and the C1q component of complement, such that the antibodies lack (or have substantially reduced) effector function. Antibodies having such modified Fc regions induce little or no antibody-dependent cell-mediated cytotoxicity (ADCC) and/or complement-mediated lysis when administered to a mammal, compared to unmodified antibodies. Assays to identify antibodies lacking effector function are known in the art. See, e.g., U.S. Pat. Nos. 6,194,551, 6,528,624 and 5,624,821; European Patent No. EP 0 753 065 B1; and PCT Publication WO 00/42072.

The CD16A binding protein of the invention may include a human IgG₁ Fc domain comprising one or more amino acid substitutions or deletions (relative to the parental naturally occurring IgG₁) that result in a reduced interaction between the Fc domain of the binding protein and FcγRIIA and/or FcγRIIIA (e.g., to minimize potential activation of macrophages and/or minimize neutrophil diminution) and/or increased binding of the Fc region to FcγRIIB (e.g., to increase FcγRIIB-mediated inhibition of effector cell activation; see Bolland and Ravetch, 1999, Adv. in Immunol. 72:149). Specific mutations effecting the desired changes in binding can be identified by selection using display of mutant Fc libraries expressed on the surface of microorganisms, viruses or mammalian cells, and screening such libraries for mutant Fc variants having the desired property or properties. In addition, the literature reports that particular residues or regions of the Fc are involved in Fcγ interactions such that deletion or mutation of these residues would be expected to result in reduced FcγR binding. The binding site on human antibodies for FcγR was reported to be the residues 233-239 (Canfield et al., 1991, J. Exp. Med. 173:1483-91; Woof et al., 1986, Mol. Imm. 23:319-30; Duncan et al., 1988, Nature 332:563). The crystal structure of FcγRIII complexed with human IgG1 Fc revealed potential contacts between the receptor and its ligand and also revealed that a single FcγRIII monomer binds to both subunits of the Fc homodimer in an asymmetric fashion. Alanine-scanning mutagenesis of the Fc region confirmed the importance of most of the predicted contact residues (Shields et al., 2001, J. Biol. Chem. 276:6591-6604).

Exemplary Fc region mutations include, for example, L235E, L234A, L235A, and D265A, which have been shown to have low affinity for all FcR, into Cγ-1 (Clynes et al., 2000, Nat. Med. 6:443-46; Alegre et al., 1992, J. Immunol. 148:3461-68; Xu et al., 2000, Cell Immunol. 200:16-26). Additional Fc region modifications purported to affect FcR binding are described in WO 00/42072 (e.g., “class 4” Fc region variants) and WO 02/061090.

Fc binding to FcγRIIA and FcγRIIIA or other proteins can be measured by any of a number of methods, including ELISA to measure binding to isolated recombinant FcγR and RIA or FACS to measure binding to cells. Immune complexes and heat aggregated or chemically crosslinked Fc or IgG can be used to test affinity for FcRs in such assays. In one embodiment, immune complexes are produced by expressing an Fc in the context of an Fab with affinity for an antigen (such as fluorescein) and mixing the antibody and antigen to form an immune complex.

E. Fc Regions with Reduced Binding to Fc Effector Ligands Due to Aglycosylation or Changes in Glycosylation

As discussed above, in CD16A binding proteins of the invention that comprise Fc domains (e.g., anti-CD16A monoclonal antibodies) the Fc domain can be modified to achieve desired properties. In a particular aspect, the invention provides a CD16A binding protein, such as a human or humanized anti-CD16A monoclonal antibody, comprising an Fc region that is not glycosylated. In some embodiments, the aglycosylated antibodies are produced my modification of the Fc region. Examples of such modifications include but are not limited to a substitution at position 297 with alanine or glutamine; or a substitution at position 298 with proline or asparagine; or a substitution at position 299 with any amino acid except serine or threonine. In other embodiments, the aglycosylated antibodies of the invention may be produced in a cell line that has a mutation in its glycosylation pathway. Such cell lines are known to those skilled in the art and include for example fuc6. As demonstrated in Example 10, infra, the inventors have discovered that, unexpectedly, administration of anti-CD16A antibodies with altered effector function (aglycosylated antibodies) protects against autoimmune disorders without inducing acute severe neutropenia. On the basis of this discovery, therapeutic anti-CD16A antibodies can be designed to protect against autoimmune diseases without inducing dangerous side effects.

In one embodiment, the invention provides a CD16A binding protein comprising an Fc region derived from human IgG₁, where the amino acids corresponding to position 297 of the C_{H}2 domains of the Fc region are aglycosyl. The terms “aglycosyl” or “aglycosylated,” when referring to an Fc region in its entirety, a specific region within the Fc region, or a specific amino acid residue in the Fc region, mean that no carbohydrate residues are attached to the specified region or residue.

The invention encompasses a CD16A binding protein, such as a human or humanized anti-CD16A monoclonal antibody, comprising an Fc region that is not glycosylated. As used herein an Fc region which is “not glycosylated” (or “aglycosylated”) encompasses Fc regions wherein the entire Fc region contains no glycosylation sites, or wherein a specific region within the Fc region is not glycosylated, or wherein a specific residue within the Fc region is not glycosylated.

The invention encompasses a CD16A binding protein, such as a human or humanized anti-CD16A monoclonal antibody, comprising an Fc region that is not glycosylated. In one specific embodiment, the invention provides a CD16A binding protein comprising an Fc region derived from human IgG₁, where the amino acid corresponding to position 297 of the C_{H}2 domains of the Fc region are aglycosyl (herein referred to a GMA-161). In another embodiment, the invention provides a CD16A binding protein that competes for binding with the GMA-161 and/or binds to the same epitope of CD16A as GMA-161. The present invention also encompasses molecules comprising an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of GMA-161.

The present invention also encompasses antibodies or fragments thereof comprising an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, or at least 99% identical to the amino acid sequence of the variable heavy chain and/or light chain of GMA-161. The present invention further encompasses antibodies or fragments thereof, said antibodies or antibody fragments comprising an amino acid sequence of one or more CDRs that is at least 45%, at least 50%, at least 55%, at least 60%, at least 65%, at least 70%, at least 75%, at least 80%, at least 85%, at least 90%, at least 95%, or at least 99% identical to the amino acid sequence of one or more CDRs of GMA-161. The determination of percent identity of two amino acid sequences can be determined by any method known to one skilled in the art, including BLAST protein searches.

In one aspect, the CD16A binding protein comprises both a V_{H} domain and a V_{L} domain, as described above (which may be prepared by coexpression of polynucleotides encoding heavy and light chains). Optionally the humanized heavy chain variable region comprises or consists of a sequence set forth in SEQ ID NOs: 109 or 120 and/or a humanized light chain variable region comprises a sequence set forth SEQ ID NOs: 96 or 98. For example, in exemplary embodiments, the binding protein has a heavy chain variable region having the sequence of SEQ ID NO:109 or 120 and a light chain variable region having the sequence of SEQ ID NO: 96 or 98. In another exemplary embodiment, the binding protein has a heavy chain variable region having the sequence of SEQ ID NO:109 and light chain variable regions having the sequence of SEQ ID NO:96. In another exemplary embodiment, the binding protein has a heavy chain variable region having the sequence of SEQ ID NO:109 and light chain variable regions having the sequence of SEQ ID NO:98. In another exemplary embodiment, the binding protein has a heavy chain variable region having the sequence of SEQ ID NO:120 and light chain variable regions having the sequence of SEQ ID NO:98. In another exemplary embodiment, the binding protein has a heavy chain variable region having the sequence of SEQ ID NO:120 and light chain variable regions having the sequence of SEQ ID NO:98.

In one embodiment, a CD16A binding protein may comprise a heavy chain variable domain sequence that is the same as, or similar to, the V_{H} domain as set forth in SEQ ID NO: 109 or 120. In one embodiment, the invention provides a CD16A binding protein comprising a V_{H} domain with a sequence that differs from the V_{H} domain of SEQ ID NO: 109 or 120 by zero, one, or more than one amino acid modifications. In other embodiments, the invention provides a CD16A binding protein that is at least about 80% identical, often at least about 90%, and sometimes at least about 95% identical, or at least about 98% identical to the V_{H} sequence of SEQ ID NO: 109 or 120.

In other embodiments, the V_{H} domain may have a sequence that differs from that of SEQ ID NO: 109 or 120 by at least one, at least two, at least three, at least four 4, at least five, or at least six of the modifications as set forth herein. In certain embodiments, the degree of sequence identity with the V_{H} domain is at least about 80%, at least about 90%, at least about 95% or at least about 98%.

In one embodiment, a CD16A binding protein may comprise a light chain variable domain sequence that is the same as, or similar to, the V_{L} domain as set forth in SEQ ID NO: 96 or 98. In one embodiment, the invention provides a CD16A binding protein comprising a V_{L} domain with a sequence that differs from the V_{L} domain of SEQ ID NO: 96 or 98 by zero, one, or more than one amino acid modification. In other embodiments, the invention provides a CD16A binding protein that is at least about 80% identical, often at least about 90%, and sometimes at least about 95% identical, or at least about 98% identical to the V_{L} sequence of SEQ ID NO: 96 or 98.

In other embodiments, the V_{L} domain may have a sequence that differs from that of SEQ ID NO: 96 or 98 by at least one, at least two, at least three, at least four 4, at least five, or at least six of the modifications as set forth herein. In certain embodiments, the degree of sequence identity with the V_{L} domain is at least about 80%, at least about 90%, at least about 95% or at least about 98%.

Human IgG antibodies that are aglycosylated show decreased binding to Fc effector ligands such as Fc receptors and C1q (see, e.g., Jefferis et al., 1995, Immunology Letters 44:111-17; Tao et al., 1989, J. of Immunology, 143:2595-2601; Friend et al., 1999, Transplantation 68:1632-37; Radaev and Sun, 2001, J. of Biological Chemistry 276:16478-83; Shields et al., 2001, J. of Biological Chemistry 276:6591-6604, and U.S. Pat. No. 5,624,821). Without intending to be bound by a particular mechanism, it is believed that the aglycosylation of the amino acid at position 297 of the Fc domains of CD16A binding proteins described herein results in reduced binding to CD16A and the C1q component of complement. Such aglycosylated antibodies lack effector function.

In human IgG1 constant regions, the residue at position 297 is asparagine. In one embodiment of the present invention, the residue at, or corresponding to, position 297 of the Fc region of the CD16A binding protein is other than asparagine. Substitution of another amino acid residue in the place of asparagine eliminates the N-glycosylation site at position 297. Substitution of any amino acid residues which will not result in glycosylation upon expression of the CD16A binding protein in a mammalian cell is appropriate for this embodiment. For instance, in some embodiments of the invention, the amino acid residue at position 297 is glutamine or alanine. In some embodiments, the amino acid residue at position 297 is cysteine, which is optionally linked to PEG.

In other embodiments of the invention, the residue at position 297 may or may not be asparagine, but is not glycosylated. This can be accomplished in a variety of ways. For example, amino acid residues other than the asparagine at position 297 are known to be important for N-linked glycosylation at position 297 (see Jefferis and Lund, 1997, Chem. Immunol. 65:111-28), and the substitution of residues at positions other than position 297 of the C_{H}2 domain can result in a CD16A binding protein aglycosylated at residue 297. For illustration and not limitation, a residue at position 299 in the C_{H}2 domain that is other than threonine or serine will result in an antibody that is aglycosylated at position 297. Similarly, substitution of the amino acid at position 298 with proline will produce an antibody with an aglycosylated amino acid at position 297. In other embodiments of the invention, Fc domains of IgG₂ or IgG₄ are used rather than IgG₁ domains.

Modification of the amino acid residues of CD16A binding proteins is well within the ability of the ordinarily skilled practitioner, and can be achieved by mutation of a polynucleotide encoding the binding protein or portion thereof. The CD16A binding protein comprising an IgG-derived Fc region need not necessarily be mutated at the amino acid level to be aglycosylated. Binding proteins aglycosylated at position 297 of the IgG-derived Fc region can be produced by expressing the CD16A binding protein in certain cells (e.g., E. coli; see PCT publication WO 02061090A2), cell lines or under certain cell culture growth conditions where glycosylation at Asn 297 does not take place. Alternatively, carbohydrate groups may be removed from a CD16A binding protein following expression of the protein, e.g., enzymatically. Methods for removing or modifying carbohydrate groups on proteins are known and include use of endoglycosidases and peptide:N-glycosidases.

It will be apparent that a variety of methods can be used to modify the Fc region of a CD16A binding protein to change its properties. Accordingly, unless otherwise specified, as used herein the term “modifying” in the context of modifying the Fc region of a CD16A binding protein includes modifying the protein itself directly, modifying the polynucleotide that encodes the protein and/or modifying or selecting a suitable expression system for production of the protein.

In addition to CD16A binding proteins that are aglycosylated at the position corresponding to arginine 297, variants with reduced binding to Fc effector ligands due to only partial removal, or modification, of the carbohydrate at that position may be used in the present invention. For example, the Fc region can be modified to include a non-naturally occurring carbohydrate that does not bestow binding protein with effector function. As used herein, a “modified Fc region” is an Fc region that has been derived from a parent Fc region, but which differs in glycosylation pattern from the parent Fc region.

In some embodiments, the invention encompasses methods of modifying the carbohydrate content of an antibody of the invention by modifying, e.g., deleting a glycosylation site. Methods for modifying the carbohydrate content of antibodies are well known in the art and encompassed within the invention, see, e.g., U.S. Pat. No. 6,218,149; EP 0 359 096 B1; U.S. Publication No. 2002/0028486; WO 03/035835; U.S. Publication No. 2003/0115614; U.S. Pat. No. 6,218,149; U.S. Pat. No. 6,472,511; all of which are incorporated herein by reference in their entirety. In other embodiments, the invention encompasses methods of modifying the carbohydrate content of an antibody of the invention by deleting one or more endogenous carbohydrate moieties of the antibody. In a specific embodiment, the invention encompasses shifting the glycosylation site of the Fc region of an antibody, by modifying positions adjacent to 297.

F. Production of CD16A Binding Proteins

CD16A binding proteins of the invention can be produced using a variety of methods well known in the art, including de novo protein synthesis and recombinant expression of nucleic acids encoding the binding proteins. The desired nucleic acid sequences can be produced by recombinant methods (e.g., PCR mutagenesis of an earlier prepared variant of the desired polynucleotide) or by solid-phase DNA synthesis. Usually recombinant expression methods are used. In one aspect, the invention provides a polynucleotide that comprises a sequence encoding a CD16A binding protein disclosed herein or a CD16A binding fragment thereof, for example a sequence encoding a V_{L} or V_{H} described herein, or antibody heavy chain or light chain described herein. Because of the degeneracy of the genetic code, a variety of nucleic acid sequences encode each immunoglobulin amino acid sequence, and the present invention includes all nucleic acids encoding the binding proteins described herein.

Recombinant expression of antibodies is well known in the art and can be carried out, for example, by inserting nucleic acids encoding light and heavy chain variable regions, optionally linked to constant regions, into expression vectors. Expression vectors typically include control sequences such as a promoter, an enhancer, and a transcription termination sequence to which DNA segments encoding polypeptides (e.g., immunoglobulin chains) are operably linked to ensure the expression of immunoglobulin polypeptides. Expression vectors are typically replicable in the host organisms either as episomes or as an integral part of the host chromosomal DNA. The light and heavy chains can be cloned in the same or different expression vectors.

Immunoglobulin light and heavy chains are expressed using standard methods. A multiple polypeptide chain antibody or antibody fragment species can be made in a single host cell expression system wherein the host cell produces each chain of the antibody or antibody fragment and assembles the polypeptide chains into a multimeric structure to form the antibody or antibody fragment in vivo. See e.g., Lucas et al., 1996, Nucleic Acids Res., 24:1774-79. When heavy and light chains are cloned on separate expression vectors, the vectors are co-transfected to obtain expression and assembly of intact immunoglobulins. Alternatively, recombinant production of antibody heavy and light chains in separate expression hosts followed by assembly of antibody from separate heavy and light chains in vitro is known. See, e.g., U.S. Pat. No. 4,816,567 and Carter et al., 1992, Bio/Technology 10:163-67.

The CD16A binding proteins are conveniently expressed in prokaryotic or eukaryotic cells. Useful hosts for antibody expression include bacteria (see, e.g., PCT publication WO 02/061090), yeast (e.g., Saccharomyces), insect cell culture (Putlitz et al., 1990, Bio/Technology 8:651-54), plants and plant cell cultures (Larrick and Fry, 1991, Hum. Antibodies Hybridomas 2:172-89), and mammalian cells. Methods for expression are well known in the art. For example, in E. coli, vectors using the lac promoter to drive expression of heavy and light chains fused to various prokaryotic secretion signal sequences such as pelB have resulted in successful secretion of scFv and Fab fragments into the periplasmic space or into the culture medium (Barbas et al., 1991, Proc. Natl. Acad. Sci. U.S.A. 88:7978-82). A vector derived from pET25b in which the lac promoter has been inserted in place of the T7 promoter may be used.

Mammalian cells are especially useful for producing CD16A binding proteins, including tetrameric antibodies or fragments thereof. A number of suitable host cell lines capable of secreting intact heterologous proteins are known, and include CHO cell lines, COS cell lines, HeLa cells, L cells and myeloma cell lines. Expression vectors for mammalian cells can include expression control sequences, such as an origin of replication, a promoter, an enhancer, ribosome binding sites, RNA splice sites, polyadenylation sites, and transcriptional terminator sequences. Examples of expression control sequences are promoters derived from endogenous genes, cytomegalovirus, SV40, adenovirus, bovine papillomavirus, and the like. In one embodiment, binding proteins are expressed using the CMV immediate early enhancer/promoter in the vector pCDNA3.1 or a similar vector. To facilitate secretion, the genes can be fused to a gene cassette containing the signal sequence of a mouse V_{H} gene described by Orlandi et al., 1989, Proc. Natl. Acad. Sci. U.S.A. 86:3833-37, which has been widely used for high-level secretion of immunoglobulins.

The vectors containing the DNA segments encoding the polypeptides of interest can be transferred into the host cell using routine methods, depending on the type of cellular host. For example, calcium chloride transfection is commonly utilized for prokaryotic cells, whereas calcium phosphate treatment, electroporation, lipofection, biolistics or viral-based transfection may be used for other cellular hosts. Other methods used to transform mammalian cells include the use of polybrene, protoplast fusion, liposomes, electroporation, and microinjection (see generally, Sambrook et al., supra). For transient expression, cells, e.g., HEK293, can be co-transfected with separate heavy and light chain expression vectors using a cationic lipid (e.g., LIPOFECTAMINE™ 2000, Invitrogen). This method can achieve expression levels of 10-20 mg/l of IgG in conditioned medium after 3 days. The cells can then be re-fed and similar quantities harvested after 3 more days. It will be appreciated that, for some uses, the cells expressing CD16A binding proteins can be maintained in medium containing FBS screened for very low levels of bovine IgG, or, alternatively, in serum-free medium.

In addition to expression of tetrameric antibodies, single chain antibodies, antibody fragments, and other CD16A binding proteins can be prepared. For example, immunoglobulin fragments can be prepared by proteolytic digestion of tetrameric antibodies, or more often, by recombinant expression of truncated antibody constructs. Usually, single chain V region (“scFv”) constructs are made by linking V_{L} and/or V_{H} domain using a short linking peptide (see, e.g., Bird et al., 1988, Science 242:423-26; U.S. Pat. Nos. 4,946,778; 5,455,030; 6,103,889; and 6,207,804).

Once expressed, the binding proteins can be purified using procedures well known in the art, including ammonium sulfate precipitation, affinity chromatography, gel electrophoresis and the like (see, generally, Harris and Angal, 1990, PROTEIN PURIFICATION APPLICATIONS, A PRACTICAL APPROACH Oxford University Press, Oxford, UK; and Coligan et al., supra). In one embodiment, purification is accomplished by capturing the antibody using a high flow rate protein A resin such as Poros A (Perceptive Biosystems, Inc), and elution at low pH, followed by size exclusion chromatography to remove any traces of aggregate present. Since FcγRIIIA binds preferentially to aggregated IgG, removal of aggregates will be desirable for certain applications. The binding proteins can be purified to substantial purity if desired, e.g., at least about 80% pure, often at least about 90% pure, more often least about 95%, or at least about 98% pure. In this context, the percent purity is calculated as a weight percent of the total protein content of the preparation, and does not include constituents which are deliberately added to the composition after the binding protein is purified.

CD16A binding proteins can be modified after expression. For example, derivation of antibodies with polyethylene glycol (“pegylation”) is reported to increase residence time (half-life and stability) and reduce immunogenicity in vivo without alteration of biological activity. See, e.g., Leong et al., 2001, Cytokine 16:106-19; Koumenis et al., 2000, Int. J. Pharm. 198:83-95; U.S. Pat. No. 6,025,158. CD16A binding proteins can be conjugated to a detectable label or ligand (e.g., a radioisotope or biotin). Other modifications are well known in the art and are also contemplated.

G. Properties of CD16A Binding Proteins

In certain embodiments, CD16A binding proteins having properties as described below are used in the methods of the invention.

i) Binding Affinity

CD16A binding proteins can be described by reference to their binding properties and biological activity. In various embodiments, the binding constant for the interaction of a CD16A binding protein of the invention and CD16A is between 0.1 and 5 nM, less than about 2.5 nM, less than about 1 nM, or less than about 0.5 nM. Usually the binding protein binds CD16A with an affinity that is within 4-fold, optionally within 2-fold, of the binding affinity exhibited under similar conditions by 3G8 or the chimeric antibody comprising the heavy chain Ch3G8VH and the light chain Ch3G8VL as described herein below. In an embodiment, the binding affinity for CD16A is greater than that of 3G8. In an alternative embodiment, the binding affinity for CD16B is no greater than, and preferably less than, 3G8 or the chimeric antibody Ch3G8.

Binding can be measured using a variety of methods, including ELISA, biosensor (kinetic analysis), and radioimmunoassay (RIA). ELISA is well known (see, Harlow and Lane, supra, and Ausubel et al., supra) and can be carried out using conditioned medium containing binding proteins or, alternatively, with purified antibodies. The concentration of antibody that results in 50% apparent maximal binding provides an estimate of antibody Kd.

Binding can also be detected using a biosensor assay, which also provides information on the kinetic and equilibrium properties of antibody binding to FcγRIIIA. An exemplary biosensor assay uses the BIAcore system (Malmqvist et al., 1997, Curr. Opin. Chem. Biol. 1:378-83). The BIAcore system relies on passing analyte over a sensor chip onto which the ligand (e.g., CD16A) is immobilized. The binding of the analyte can be measured by following surface plasmon resonance (SPR) signal, which changes in direct proportion to the mass bound to the chip. A fixed concentration of analyte is passed over the chip for a specific amount of time, allowing for the measurement of the association rate, k(on). Following this phase, buffer alone is passed over the chip and the rate at which the analyte dissociates from the surface, k(off) can be measured. The equilibrium dissociation constant can be calculated from the ratio of the kinetic constants; Kd=k(on)/k(off).

A radioimmunoassay (RIA) can be used to measure the affinity of antibodies for FcγRIII-bearing cells, and to measure inhibition of IgG complexes to cells by these antibodies. In an exemplary assay, ¹²⁵I labeled binding protein is prepared and specific radioactivity of the protein determined. Labeled binding protein and cells are mixed for several hours, the cells and bound material are separated from the unbound material by centrifugation, and the radioactivity in both compartments is determined. A direct binding format is used to determine the Kd of, and the number of binding sites for, iodinated binding protein using Scatchard analysis of the binding data. Controls containing an excess of cold (unlabeled) binding protein competitor can be included to ensure the results reflect specific interactions. Examples of suitable cells include (1) NK cells or macrophages derived from normal human peripheral blood lymphocytes; (2) Cells obtained from huCD16A transgenic mice (Li et al., 1996 J. Exp. Med. 183:1259-63); (3) mammalian cell lines expressing the extracellular portion of CD16A fused to the transmembrane and intracellular domain of RII or another receptor (such as CD8 or LFA-3); (4) mammalian cell lines (e.g., CHO, HEK-293, COS) transfected transiently or stably with CD16A expression vectors (and optionally coexpressing gamma chain for optimal expression receptor expression).

Examples of expression vectors useful for expression of CD16A and other polypeptides for use in binding assays include mammalian expression vectors (e.g., pCDNA 3.1 or pCI-neo) that contain a strong promoter/enhancer sequence (e.g., CMV immediate early) and a polyadenylation/transcription termination site flanking a polylinker region into which the CD16A gene is introduced. Usually the vector includes a selectable marker such as a neomycin resistance gene.

In one embodiment, the CD16A expressed for use in assays has the sequence:

(SEQ ID NO:116)

MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLFGSKNVSSETVNITITQGLAVSTIS
SFFPPGYQVSFCLVMVLLFAVDTGLYFSVKTNIRSSTRDWKDHKFKWRKD
PQDK.

CD16A with the sequence:

(SEQ ID NO:117)

MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHIGWLLLQAPRWVFKEEDPIHLRCHSWKNTALHKVTYLQNGKGRKY
FHHNSDFYIPKATLKDSGSYFCRGLVGSKNVSSETVNITITQGLAVSTIS
SFFPPGYQVSFCLVMVLLFAVDTGLYFSVKTNIRSSTRDWKDHKFKWRKD
PQDK

can also be used.

Additional CD16A variants and substitutes will be known to, or readily discernible from the scientific literature by, the ordinarily skilled artisan.

Competitive assay formats can be used to measure the ability of a CD16A binding protein to inhibit binding of another molecule to the receptor. For example, in one competitive assay format a fixed amount of labeled 3G8 is mixed with varying amounts of either unlabeled 3G8, CD16A binding protein or an irrelevant IgG (control) and added to FcγRIIIA expressing cells. After incubation and separation of the cell-bound material from the material free in solution, the amount of bound labeled 3G8 (and/or optionally also the unbound labeled 3G8) is determined. The concentration of unlabeled mAb which results in a 50% decrease in the binding of labeled 3G8 (IC50) is then determined from this data.

ii) Blocking Immune Complex Binding to FcγRIIIA

Another characteristic of the CD16A binding proteins of the invention is the ability to inhibit binding of immune complexes to CD16A (“IC Blocking Activity”). Usually the binding protein has IC Blocking Activity that is within 4-fold, preferably within 2-fold, of the activity exhibited under similar conditions by 3G8 or the chimeric antibody, Ch3G8, described herein.

Assays for measuring ability of an antibody to block binding of complexed IgG to CD16A are known. See, e.g., Knapp et al., 1989, LEUKOCYTE TYPING IV, Oxford University Press, Oxford, p. 574-97; and Edberg and Kimberly, 1997, J. Immunol. 159:3849-57. One suitable assay is an RIA assay with the format described above for the competitive assay, but substituting ¹²⁵I-labeled aggregated irrelevant human IgG₁ for the ¹²⁵I-labeled 3G8 used in the competitive assay described above.

The invention provides a method of inhibiting the binding of IgG antibodies to CD16 on a cell by contacting the cell with a CD16A binding protein under conditions in which the CD16A binding protein binds the FcγRIII on the cell. The contacting can be in vivo (e.g., by administering the binding protein in a mammal) or in vitro (e.g., by addition of antibodies to cultured cells expressing the FcγRIII). IgG antibodies that are inhibited from binding the FcγRIII can be administered to the animal or added to a culture medium before or after addition or administration of the binding protein, or may be present in an animal normally or in response to a disease state. In one embodiment, the CD16 on the surface of the cell is CD16A.

iii) In Vivo Protection Against Platelet Depletion

The ability of the CD16A binding proteins of the invention to reduce deleterious immune responses can be assessed in a variety of animal models. An exemplary model system is a mouse model for idiopathic thrombocytopenic purpura (ITP) (see, Oyaizu et al., 1988, J Exp. Med. 167:2017-22; Mizutani et al. 1993, Blood 82:837-44). See Example 9, infra. Other suitable models are known in the art. Other animal models include rodent models of inflammatory diseases described in, for example, Current Protocols in Immunology (in some cases modified by using animals transgenic for human CD16A). Transgenic mice can be made using routine methods or can be purchased from commercial sources (e.g., Taconic Inc., German Town, N.Y.).

An example of a procedure suitable for assessing the ability of a CD16A binding protein to provide protection from platelet depletion in a mouse model is described in Example 8, infra. CD16A binding proteins can be administered to muFcγRIII−/−, huFcγRIIIA transgenic mice at a variety of concentrations, and ITP subsequently induced in the mice (e.g., by administering the 6A6 or chimeric 6A6 antibody) to the mice. At timed intervals after the administration of 6A6/ch6A6, the mice are bled and the platelet counts are determined. Optionally, the IC₅₀ for each molecule is then determined at the time point where maximal platelet depletion is observed in the negative control group. Based on the results of Example 8 and on prior studies, maximum depletion occurred 2-6 hr after 6A6 administration. IC₅₀s are determined graphically, using a curve-fitting program such as the four-parameter fit provided in the SigmaPlot program. Statistically significant inhibition of depletion of platelets after administration of 6A6 in the treatment group compared to the untreated group and a group administered an identical formulation of an irrelevant, isotype matched mAb is indicative of the desired biological activity.

Experiments in which protection by CD16A binding proteins was assayed are described in the Examples, infra. Preparations of recombinant mouse 3G8 produced in HEK-293 cells, chimeric 3G8 with human IgG1 or IgG2 constant domains (ch3G8-γ1 produced in HEK-293 and CHO-K1 cells, and ch3G8-γ2 produced in HEK-293 cells), and a ch3G8-γ1 variant (ch3G8-γ1 D265A) did not provide significant protection. Murine 3G8, produced from the hybridoma, and a chimeric version of 3G8 containing an aglycosylated human G1 constant region (Ch3G8-G1 N297Q), produced in HEK-293 cells, were able to protect animals from platelet depletion in the mouse model. As shown in Example 10, 11 and 15-17, infra, Ch3G8 N297Q and aglycosylated humanized antibodies protected against platelet depletion in the ITP mouse model. Although not intending to be bound by a particular theory, one possibility is that since ch3G8 N297Q is largely devoid of effector function, it is more efficient than ch3G8 in protecting mice against ITP. Thus, these data suggest that anti-CD16A antibodies without effector function (e.g., aglycosylated antibodies) have advantages compared to some glycosylated (e.g., glycosylated recombinant) antibodies. Further, as described in the examples, administration of aglycosylated anti-CD16A antibody to muFcγRIII−/−, huFcγRIIIB transgenic mice did not result in neutrophil depletion in the blood, spleen, and bone marrow. Without intending to be bound by a particular theory, there are several possible explanations for these unexpected results. Protein glycosylation is known to vary in different cell lines, especially those from different species. A difference in the nature of the carbohydrate attached to the antibody constant region as a consequence of expression in different cell types may be responsible for the difference in activity, i.e., if the lack of activity results in part from effector cell activation caused by ch3G8 binding to Fc receptors (or complement) via the antibody Fc region in a glycosylation-dependent manner. Alternatively, recombinant murine and ch3G8 may contain other post-translational modifications that affect activity and which can be eliminated by using different cell lines to express the CD16A binding proteins. It is possible that a combination of isotype and/or isotype containing mutations to eliminate effector function may provide similar protective effects as elimination of the carbohydrate on the Fc.

5. Methods of Treatment

A number of diseases and conditions characterized by a deleterious immune response can be treated using the binding proteins of the invention, i.e., a CD16A binding protein as described herein (e.g., comprising a V_{L} and/or V_{H} sequence as disclosed herein and, optionally, a Fc region modified as disclosed herein to have a reduced effector function). In one embodiment, the binding protein is administered to a subject with an autoimmune disease (i.e., a disease characterized by the production of autoantibodies). It is believed that pathogenic IgG antibodies observed in autoimmune diseases are either the pathogenic triggers for these diseases or contribute to disease progression and mediate disease through the inappropriate activation of cellular Fc receptors. Aggregated autoantibodies and/or autoantibodies complexed with self antigens (immune complexes) bind to activating FcRs, thereby triggering the pathogenic sequelae of numerous autoimmune diseases (which occur in part because of immunologically mediated inflammation against self tissues). Without intending to be bound by a particular mechanism of action, the CD16A binding proteins described herein interfere with and reduce the interaction of the autoimmune antibodies and FcγRIII receptors.

Examples of autoimmune diseases that can be treated include, without limitation, idiopathic thrombocytopenic purpura (ITP), rheumatoid arthritis (RA), systemic lupus erythrematosus (SLE), autoimmune hemolytic anemia (AHA), scleroderma, autoantibody triggered urticaria, pemphigus, vasculitic syndromes, systemic vasculitis, Goodpasture's syndrome, multiple sclerosis (MS), psoriatic arthritis, ankylosing spondylitis, Sjögren's syndrome, Reiter's syndrome, Kawasaki's disease, polymyositis and dermatomyositis. Other examples of diseases or conditions that can be treated according to the invention also include any diseases susceptible to treatment with intravenous immunoglobulin (IVIG) therapy (e.g., allergic asthma). Thus, the treatment of autoimmune diseases heretofore treated by IVIG therapy (in one embodiment, a condition other than ITP) is contemplated. While detailed understanding of the mechanism of action of IVIG has not been established, it is proposed that modulating the activity of cellular FcγRs plays a role in its in vivo efficacy. The protective activity of IVIG may rely on the small percentage of dimeric or polymeric IgG present in the preparation. The specificity of the FcγRIII pathway in coupling cytotoxic and immune complex antibodies to effector responses and the ability to directly block this pathway with a mAb strongly suggests that an anti-FcγRIII antibody will have enhanced activity relative to IVIG.

Other examples of autoimmune disorders that may be treated by administering the antibodies of the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Mknikre's disease, mixed connective tissue disease, multiple sclerosis, type 1 or immunemediated diabetes mellitus, myasthenia gravis, pemphigus vulgaris, pernicious anemia, polyarteritis nodosa, polychrondritis, polyglandular syndromes, polyrnyalgia rheumatics, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythernatosus, takayasu arteritis, temporal arteristisl giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory-bowel disease, chronic obstructive pulmonary disease (COPD), allergic disorders, septic shock, pulmonary fibrosis, undifferentiated spondyloarthropathy, undifferentiated arthpathy, arthritis, inflammatory osteolysis, and chronic inflammation resulting from chronic viral or bacteria infections. Some autoimmune disorders are associated with an inflammatory condition. Thus, there is overlap between what is considered an autoimmune disorder and an inflammatory disorder. Therefore, some autoimmune disorders may also be characterized as inflammatory disorders. Examples of inflammatory disorders which can be prevented, treated or managed in accordance with the methods of the invention include, but are not limited to, asthma, encephilitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacteria infections.

A reduction in a deleterious immune response can be detected as a reduction in inflammation. In a specific embodiment, an antibody reduces the inflammation in an animal by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the inflammation in an animal in the not administered said antibody. Alternatively, a reduction in a deleterious immune response can be detected as a reduction in symptoms characteristic of the condition being treated (e.g., a reduction in symptoms exhibited by a subject suffering from an autoimmune condition), or by other criteria that will be easily recognized by physicians and experimentalists in the field of autoimmunity. It will be apparent that, in many cases, specific indicia of reduction will depend on the specific condition being treated. For example, for illustration and not limitation, a reduction in a deleterious immune response in a subject with ITP can be detected as a rise in platelet levels in the subject. Similarly, a reduction in a deleterious immune response in a subject with anemia can be detected as a rise in RBC levels in the subject. A clinician will recognize significant changes in platelet or RBC levels, or other responses following treatment.

The deleterious immune response is optionally due to idiopathic thrombocytopenic purpura resulting from the administration of an antiplatelet antibody, optionally murine monoclonal antibody 6A6, to a muFcγRIII−/−, huFcγRIIIA transgenic mouse.

In one aspect, the invention provides a method for treating an autoimmune disease, such as ITP, by administering a CD16A binding protein that is largely devoid of effector function. In an embodiment, the CD16A binding protein comprises Fc regions derived from human IgG. In an embodiment, the Fc regions are aglycosyl. In an embodiment, position 297 of each of the CH2 domains is a residue other than asparagine or proline. In one aspect, the binding protein comprises a variable region sequence as described elsewhere herein. However, as discussed herein, the compositions and treatment methods of the invention are not limited to specific CD16A binding proteins derived from murine mAb 3G8, but are applicable to CD16A binding proteins in general. In an embodiment, the CD16A binding protein is a tetrameric antibody protein having two light chains and two heavy chains.

In a related aspect, the invention provides methods of reducing a deleterious immune response in a mammal without significantly reducing neutrophil levels or inducing nentropenia (e.g., severe neutropenia or moderate neutropenia) by administering to the mammal a therapeutically effective amount of a pharmaceutical composition comprising a CD16A binding protein described herein. In an embodiment, the mammal is human. In an embodiment, the mammal is a nonhuman mammal (e.g., mouse) comprising one or more human transgenes.

For therapeutic applications, the binding proteins of the invention are formulated with a pharmaceutically acceptable excipient or carrier, e.g., an aqueous carrier such as water, buffered water, 0.4% saline, 0.3% glycine and the like, optionally including other substances to increase stability, shelf-life or to approximate physiological conditions (sodium acetate, sodium chloride, potassium chloride, calcium chloride, sodium lactate, histidine and arginine). For administration to an individual, the composition is preferably sterile, and free of pyrogens and other contaminants. The concentration of binding protein can vary widely, e.g., from less than about 0.01%, usually at least about 0.1% to as much as 5% by weight. Methods for preparing parentally administrable compositions are known or apparent to those skilled in the art and are described in more detail in, for example, Remington, THE SCIENCE OF PRACTICE AND PHARMACY, 20th Edition Mack Publishing Company, Easton, Pa., 2001). The pharmaceutical compositions of the invention are typically administered by a parenteral route, most typically intravenous, subcutaneous, intramuscular, but other routes of administration can be used (e.g., mucosal, epidermal, intraperitoneal, oral, intranasal, and intrapulmonary). In a specific embodiment, the antibodies of the invention are administered intramuscularly, intravenously, or subcutaneously. The compositions of the invention may be administered to other sites of the patient's body, e.g., bone marrow, spinal cord. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985, 320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

Although not required, pharmaceutical compositions are preferably supplied in unit dosage form suitable for administration of a precise amount.

In one embodiment, CD16A binding proteins can be administered in a form, formulation or apparatus for sustained release (e.g., release over a period of several weeks or months).

In one embodiment, polynucleotides encoding CD16A binding proteins (e.g., CD16A binding protein expression vectors) are administered to a patient. Following administration, the CD16A binding protein is expressed in the patient. Vectors useful in administration of CD16A binding proteins can be viral (e.g., derived from adenovirus) or nonviral. Usually the vector will comprise a promoter and, optionally, an enhancer that serve to drive transcription of a protein or proteins. Such therapeutic vectors can be introduced into cells or tissues in vivo, in vitro or ex vivo. For ex vivo therapy, vectors may be introduced into cells, e.g., stem cells, taken from the patient and clonally propagated for autologous transplant back into the same patient (see, e.g., U.S. Pat. Nos. 5,399,493 and 5,437,994).

The compositions can be administered for prophylactic and/or therapeutic treatments. In prophylactic applications, compositions are administered to a patient prior to an expected or potential deleterious immune response. For example, idiopathic thrombocytopenic purpura and systemic lupus erythrematosus are conditions in which a deleterious immune response can be exacerbated by administration of certain medications. The CD16A binding compositions of the invention can be administered in anticipation of such medication-induced responses to reduce the magnitude of the response. In therapeutic applications, compositions are administered to a patient already suffering from a deleterious immune response in an amount sufficient to at least partially ameliorate the condition and its complications. An amount adequate to accomplish this may be a “therapeutically effective amount” or “therapeutically effective dose.” Amounts effective for these uses depend upon the severity of the condition and the general state of the patient's own immune system, but generally range from about 0.01 to about 100 mg of antibody per dose, with dosages from 0.1 to 50 mg and 1 to 10 mg per patient being more commonly used. An “inflammation reducing amount” of the binding protein can also be administered to a mammal to reduce a deleterious immune response.

The amount of the composition of the invention which will be effective in the treatment, prevention or amelioration of one or more symptoms associated with a deleterious immune response, e.g., an autoimmune disease, can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

The dosage of the compositions of the invention administered to a patient is typically about 0.1 mg/kg to about 10 mg/kg of the patient's body weight, e.g., about 0.1, about 0.2, about 0.3, about 0.4, about 0.5, about 0.6, about 0.7, about 0.8, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, and about 10 mg/kg of the patient's body weight. Preferably, the dosage administered to a patient is between about 0.1 mg/kg and about 3 mg/kg of the patient's body weight. In other embodiments the dosage of the compositions of the invention is about 0. 1, about 0.3, about 1.0 or about 3.0 mg/kg of the patient's body weight. In a most preferred embodiment the composition of the invention is administered intravenously over about 30 minutes. In other embodiments, the composition of the invention is administered intravenously over at least about 1 hour, at least about 30 minutes, or at least about 15 minutes.

The administration of the CD16A binding proteins can be administered according to the judgment of the treating physician, e.g., daily, weekly, biweekly or at any other suitable interval, depending upon such factors, for example, as the nature of the ailment, the condition of the patient and half-life of the binding protein.

Treatment of a subject with a therapeutically or prophylactically effective amount of CD16A binding proteins of the invention can include a single treatment or, preferably, can include a series of treatments. In a preferred example, a subject is treated with CD16A binding proteins of the invention in the range of between about 0.1 to about 10 mg/kg body weight, one time per week for between about 1 to about 10 weeks, preferably between about 2 to about 8 weeks, more preferably between about 3 to about 7 weeks, and even more preferably for about 4, about 5, or about 6 weeks. In other embodiments, the pharmaceutical compositions of the invention are administered once a day, twice a day, or three times a day. In other embodiments, the pharmaceutical compositions are administered once a week, twice a week, once every two weeks, once a month, once every six weeks, once every two months, twice a year or once per year. It will also be appreciated that the effective dosage of the antibodies used for treatment may increase or decrease over the course of a particular treatment.

CD16A binding proteins can be administered in combination with other treatments directed to alleviation of the deleterious immune response or its symptoms or sequelae. Thus, the binding proteins can be administered as part of a therapeutic regimen that includes co-administration of another agent or agents, e.g., a chemotherapeutic agent such as a non-steroidal anti-inflammatory drug (e.g., aspirin, ibuprofen), steroids (e.g., a corticosteroid, prednisone), immunosuppressants (e.g., cyclosporin A, methotrexate cytoxan), and antibodies (e.g., in conjunction with IVIG).

A. PHARMACEUTICAL COMPOSITIONS

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of antibodies of the invention and a pharmaceutically acceptable carrier.

In one particular embodiment, the pharmaceutical composition comprises of a therapeutically effective amount of a CD16A binding protein and a pharmaceutically acceptable carrier. In another embodiment, said pharmaceutical composition further comprises one or more additional agents.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, flee, flour, chalk, silica gel, sodium stearate, glycerol monostearate, tale, sodium chloride, dried skim milk, glycerol, propylene, glycol, water, ethanol and the like. The composition, if desired, can also contain minor amounts of wetting or -emulsifying agents, or pH buffering agents. These compositions earl take the form of solutions, suspensions, emulsion, tablets, pills, capsules, powders, sustained-release formulations and the like. The compositions of the invention may contain any excipient known in the art, such as those disclosed in Handbook of Pharmaceutical Excipients, Arthur H. Kibbe (ed., 2000), Am. Pharmaceutical Association, Washington, D.C.; the contents of which are incorporated herein by reference in entirety.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

6. Increasing the Therapeutic Efficacy of a CD16A Binding Protein

In a related aspect, the invention provides a method for increasing the therapeutic efficacy of a CD16A binding protein comprising one or more Fc domains (e.g., anti-CD16A antibodies comprising two Fc domains) by modifying the protein so that it has Fc region(s) with reduced binding to at least one Fc effector ligand compared to the original (i.e., unmodified) Fc region. For example, the Fc region can be modified so that the Fc region is not glycosylated. As described above, modification of the Fc region can be accomplished in several ways (e.g., by genetic mutation, by choice of expression system to change the Fc glycosylation pattern, and the like). In one embodiment, the Fc effector ligand is FcγRIII. In one embodiment, the Fc effector ligand is the C1q component of complement. As used in this context, a subject CD16A binding protein has increased “therapeutic efficacy” compared to a reference binding protein that induces neutropenia when administered if the subject CD16A binding protein does not induce neutropenia (or results in less severe neutropenia). For example, a CD16A binding protein that reduces the severity of a deleterious immune response (e.g., ITP or experimentally induced ITP in a mammal) and reduces neutrophil levels in the animal by x % has greater “therapeutic efficacy” than a CD16A binding protein that reduces the severity of a deleterious immune response and reduces neutrophil levels in the animal by y %, if y is greater than x, e.g. two-fold greater. In one embodiment, the protein is modified by mutation such that the modified protein is aglycosylated.

For example, the invention provides methods for producing a modified CD16A binding protein comprising a modified immunoglobulin heavy chain, the modified CD16A binding protein having greater therapeutic efficacy than a parent CD16A binding protein comprising a parent immunoglobulin heavy chain, by (i) introducing at least one mutation into a parent polynucleotide that encodes the parent immunoglobulin heavy chain to produce a modified polynucleotide that encodes the modified immunoglobulin heavy chain, the mutation introducing into the modified immunoglobulin heavy chain an amino acid substitution that changes, reduces or eliminates glycosylation in the C_{H}2 domain of the parent immunoglobulin heavy chain; and (ii) expressing the modified polynucleotide in a cell as the modified immunoglobulin heavy chain so as to produce the modified CD16A binding protein heavy chain. Optionally, the heavy chain is produced under conditions of co-expression with a light chain to produce a tetrameric antibody.

7. Eliminating Cytokine Release Syndrome

The use of therapeutic monoclonal antibodies is limited by problems of “first dose” side effects. First dose side effects, range from mild flu-like symptoms to severe toxicity, can be mild to severe, and include symptoms, such as, high fever, chills/rigors, headache, tremor, nausea/vomiting, diarrhea, abdominal pain, malaise, muscle/joint aches and pains, and generalized weakness. The first dose side effects are believed to be caused by lymphokine production and cytokine release stimulated by the Fc region of a mAb binding to and activating an FcγR on an FcγR-containing cell. Alternatively, first dose side effects may be caused by Fc region binding to complement related receptors such as C1q.

The invention thus encompasses CD16A binding proteins that reduced or eliminate at least one symptom associated with first dose side effects by reducing or eliminating binding of the Fc to one or more FcγR or by reducing or eliminating binding of the Fc to at least one complement related receptor such as C1q. Such CD16A binding proteins comprise a variant Fc region having one or more amino acid modifications, relative to a wild-type Fc region. The modification decreases or eliminates binding of the Fc to one or more FcγRs (or reduces or eliminates binding of the Fc to at least one complement related receptor such as C1q), relative to a comparable wild-type Fc region. The modification is typically an amino acid substitution. However, the modification can be an amino acid insertion and/or deletion. Typically, the modification occurs in the CH2 and/or hinge region. Alternatively, binding of Fc to one or more FcγRs can be reduced or eliminated by altering or eliminating one or more glycosyl groups on one in more Fc regions. Fc glycosylation can be altered or eliminated by methods well know in the art. For example, Fc glycosylation can be altered by producing the Fc in a cell that is deficient in fucosylation (e.g., fuc6 null cells), or eliminated by deglycosylation enzymes or an amino acid modification that alters or eliminates a glycosylation site (e.g., the N-X-S/T glycosylation site at positions 297-299 in the CH2 domain). FcγR binding can be measured using standard methods known in the art and exemplified herein. The antibodies of the invention are thus particularly useful because they have reduced or no in vivo toxicity caused by lymphokine production or cytokine release syndrome.

Methods of measuring lymphokine production and cytokine release are known and routine in the art and encompassed herein. For example, cytokine release may be measured by measuring secretion of cytokines including but not limited to TNF-α, GM-CSF, IFN-γ. See, e.g., U.S. Pat. No. 6,491,916; Isaacs et al., 2001, Rheumatology, 40: 724-738; each of which is incorporated herein by reference in its entirety. Lymphokine production may be measured by measuring secretion of lymphokines including but not limited to Interleukin-2 (IL-2), Interleukin-4 (IL-4), Interleukin-6 (IL-6), Interleukin-12 (IL-12), Interleukin-16 (IL-16), PDGF, TGF-α, TGF-β, TNF-α, TNF-β, GCSF, GM-CSF, MCSF, IFN-α, IFN-β, TFN-γ, IGF-I, IGF-II. For example, see, Isaacs et al., 2001, Rheumatology, 40: 724-738; Soubrane et al., 1993, Blood, 81(1): 15-19; each of which is incorporated herein by reference in its entirety.

As used herein, the term “Fc region” is used to define a C-terminal region of an IgG heavy chain. Although the boundaries may vary slightly, the human IgG heavy chain Fc region is defined to stretch from Cys226 to the carboxy terminus. The Fc region of an IgG comprises two constant domains, CH2 and CH3. The CH2 domain of a human IgG Fc region usually extends from amino acids 231 to amino acid 341. The CH3 domain of a human IgG Fc region usually extends from amino acids 342 to 447. The CH2 domain of a human IgG Fc region (also referred to as “Cγ2” domain) usually extends from amino acid 231-340. The CH2 domain is unique in that it is not closely paired with another domain. Rather, two N-linked branched carbohydrate chains are interposed between the two CH2 domains of an intact native IgG.

Throughout the present specification, the numbering of the residues in an IgG heavy chain is that of the EU index as in Kabat et al., Sequences of Proteins of Immunological Interest, 5^{th} Ed. Public Health Service, NH1, MD (1991), expressly incorporated herein by references. The “EU index as in Kabat” refers to the numbering of the human IgG1 EU antibody.

The “hinge region” is generally defined as stretching from Glu216 to Pro230 of human IgG1. Hinge regions of other IgG isotypes may be aligned with the IgG1 sequence by placing the first and last cysteine residues forming inter-heavy chain S-S binds in the same positions.

Examples of Fc modifications that will reduce or eliminate at least one symptom associated with first dose side effect include, but are not limited to, those having a substitution at position 233 with proline; or a substitution at position 234 with alanine, or a substitution at position 235 with alanine, or a substitution at position 234 with alanine and at position 235 with an alanine, or a substitution at position 238 with arginine; or a substitution at position 265 with alanine; or a substitution at position 265 with glutamic acid; or a substitution at position 270 with alanine; or a substitution at position 270 with asparagine; or a substitution at position 297 with alanine or glutamine; or a substitution at position 298 with proline or asparagine; or a substitution at position 299 with any amino acid except serine or threonine; or a substitution at position 265 with alanine and at position 297 with alanine; or a substitution at position 265 with alanine and at position 297 with glutamine; or a substitution at position 265 with glutamic acid and at position 297 with alanine; or a substitution at position 265 with glutamic acid and at position 297 with glutamine. The invention further encompasses the combinations of any of the variants listed herein.

The invention encompasses methods for reducing or eliminating at least one symptom associated with first dose side effect in a patient comprising administering an effective amount of one or more antibodies of the invention. The methods of the invention reduce at least one symptom associated with cytokine release syndrome including but not limited to high fever, chills/rigors, headache, tremor, nausea/vomiting, diarrhea, abdominal pain, malaise, muscle/joint aches and pains, and generalized weakness.

### 8. EXAMPLES

### Example 1

### Mouse 3G8 V_{H} and V_{L} and Chimeric Molecules Generated Therefrom

A) Mouse 3G8 V_{H} and V_{L}

The cDNA encoding the mouse 3G8 antibody light chain was cloned. The sequence of the 3G8 antibody heavy chain was provided by Dr. Jeffrey Ravetch. The amino acid sequences of the 3G8 V_{H} and V_{L} are provided in Tables 1 and 3, infra. Nucleic acid sequences encoding the variable regions are:

{3G8VH}

SEQ ID NO:1

CAGGTTACTCTGAAAGAGTCTGGCCCTGGGATATTGCAGCCCTCCCAGAC
CCTCAGTCTGACTTGTTCTTTCTCTGGGTTTTCACTGAGGACTTCTGGTA
TCGGTGTAGGCTGGATTCGTCAGCCTTCAGGGAAGGGTCTAGAGTGGCTG
GCACACATTTGGTGGGATGATGACAAGCGCTATAATCCAGCCCTGAAGAG
CCGACTGACAATCTCCAAGGATACCTCCAGCAACCAGGTATTCCTCAAAA
TCGCCAGTGTGGACACTGCAGATACTGCCACATACTACTGTGCTCAAATA
AACCCCGCCTGGTTTGCTTACTGGGGCCAAGGGACTCTGGTCACTGTCTC
TGCA
{3G8VL}

SEQ ID NO:3

GACACTGTGCTGACCCAATCTCCAGCTTCTTTGGCTGTGTCTCTAGGGCA
GAGGGCCACCATCTCCTGCAAGGCCAGCCAAAGTGTTGATTTTGATGGTG
ATAGTTTTATGAACTGGTACCAACAGAAACCAGGACAGCCACCCAAACTC
CTCATCTATACTACATCCAATCTAGAATCTGGGATCCCAGCCAGGTTTAG
TGCCAGTGGGTCTGGGACAGACTTCACCCTCAACATCCATCCTGTGGAGG
AGGAGGATACTGCAACCTATTACTGTCAGCAAAGTAATGAGGATCCGTAC
ACGTTCGGAGGGGGGACCAAGCTGGAAATAAAA

B) Chimeric Heavy Chain

To create a chimeric gene coding for expression of the mouse 3G8 VH fused to a human constant domain, the nucleic acid encoding the 3G8 VH was fused to sequences encoding a signal peptide (see Orlandi et al., 1989, Proc. Natl. Acad. Sci. U.S.A. 86:3833-37; in lower case underline below) and a human Cγ1 constant region (in lower case below) using standard techniques (including overlapping PCR amplification). To facilitate cloning, a SacI site was introduced, resulting in a single residue change in VH FR4 (ala→ser). This change in FR4 does not affect binding to CD16. The resulting nucleic acid had the sequence shown below. The region encoding the V_{H} domain is in upper case.

{ch3G8VH}

SEQ ID NO:5

gctagcatttaaacttaagcttgttgactagtgagatcacagttctctct
acagttactgagcacacaggacctcaccatgggatggagctgtatcatcc
tcttcttggtagcaacagctacaggtaaggggctcacagtagcaggcttg
aggtctggacatatatatgggtgacaatgacatccactttgcctttctct
ccacaggtgtccactccCAGGTTACCCTGAAAGAGTCTGGCCCTGGGATA
TTGCAGCCCTCCCAGACCCTCAGTCTGACTTGTTCTTTCTCTGGGTTTTC
ACTGAGGACTTCTGGTATGGGTGTAGGCTGGATTCGTCAGCCTTCAGGGA
AGGGTCTAGAGTGGCTGGCACACATTTGGTGGGATGATGACAAGCGCTAT
AATCCAGCCCTGAAGAGCCGACTGACAATCTCCAAGGATACCTCCAGCAA
CCAGGTATTCCTCAAAATCGCCAGTGTGGACACTGCAGATACTGCCACAT
ACTACTGTGCTCAAATAAACCCCGCCTGGTTTGCTTACTGGGGCCAAGGG
ACTCTGGTCACTGTGAGCTCAgcctccaccaagggcccatcggtcttccc
cctggcaccctcctccaagagcacctctgggggcacagcggccctgggct
gcctggtcaaggactacttccccgaaccggtgacggtgtcgtggaactca
ggcgccctgaccagcggcgtgcacaccttcccggctgtcctacagtcctc
aggactctactccctcagcagcgtggtgaccgtgccctccagcagcttgg
gcacccagacctacatctgcaacgtgaatcacaagcccagcaacaccaag
gtggacaagagagttgagcccaaatcttgtgacaaaactcacacatgccc
accgtgcccagcacctgaactcctggggggaccgtcagtcttcctcttcc
ccccaaaacccaaggacaccctcatgatctcccggacccctgaggtcaca
tgcgtggtggtggacgtgagccacgaagaccctgaggtcaagttcaactg
gtacgtggacggcgtggaggtgcataatgccaagacaaagccgcgggagg
agcagtacaacagcacgtaccgtgtggtcagcgtcctcaccgtcctgcac
caggactggctgaatggcaaggagtacaagtgcaaggtctccaacaaagc
cctcccagcccccatcgagaaaaccatctccaaagccaaagggcagcccc
gagaaccacaggtgtacaccctgcccccatcccgggatgagctgaccaag
aaccaggtcagcctgacctgcctggtcaaaggcttctatcccagcgacat
cgccgtggagtgggagagcaatgggcagccggagaacaactacaagacca
cgcctcccgtgctggactccgacggctccttcttcctctacagcaagctc
accgtggacaagagcaggtggcagcaggggaacgtcttctcatgctccgt
gatgcatgaggctctgcacaaccactacacgcagaagagcctctccctgt
ctccgggtaaatgagtgcggccgcgaattc

This construct was inserted into the pCI-Neo (Promega Biotech) at the NheI-EcoRI sites of the polylinker for use for expression of the chimeric heavy chain in cells.

C) Chimeric Light Chain

To create a chimeric gene coding for the mouse 3G8 V_{L} fused to a human constant domain, this 3G8 V_{L} segment was fused to a signal sequence (as for the V_{H} above; (lower case underlined) and a human C constant region (lower case)) cDNA using standard techniques, resulting in a nucleic acid with the sequence shown below:

ch3G8VL

SEQ ID NO:6

gctagctgagatcacagttctctctacagttactgagcacacaggacctc
accatgggatggagctgtatcatcctcttcttggtagcaacagctacagg
taaggggctcacagtagcaggcttgaggtctggacatatatatgggtgac
aatgacatccactttgcctttctctccacaggtgtccactccGACACTGT
GCTGACCCAATCTCCAGCTTCTTTGGCTGTGTCTCTAGGGCAGAGGGCCA
CCATCTCCTGCAAGGCCAGCCAAAGTGTTGATTTTGATGGTGATAGTTTT
ATGAACTGGTACCAACAGAAACCAGGACAGCCACCCAAACTCCTCATCTA
TACTACATCCAATCTAGAATCTGGGATCCCAGCCAGGTTTAGTGCCAGTG
GGTCTGGGACAGACTTCACCCTCAACATCCATCCTGTGGAGGAGGAGGAT
ACTGCAACCTATTACTGTCAGCAAAGTAATGAGGATCCGTACACGTTCGG
AGGGGGGACCAAGCTTGAGATCAAAcgaactgtggctgcaccatcggtct
tcatcttcccgccatctgatgagcagttgaaatctggaactgcctctgtt
gtgtgcctgctgaataacttctatcccagagaggccaaagtacagtggaa
ggtggataacgccctccaatcgggtaactcccaggagagtgtcacagagc
aggacagcaaggacagcacctacagcctcagcagcaccctgacgctgagc
aaagcagactacgagaaacacaaagtctacgcctgcgaagtcacccatca
gggcctgagctcgcccgtcacaaagagcttcaacaggggagagtgttagt
tctagagtcgactctagaggatccccgggtaccgagctcgaattc

This construct was inserted into pCI-Neo (Promega Biotech) at the NheI-EcoRI sites of the polylinker for use for expression of the chimeric light chain in cells.

D) Expression

The ch3G8VH and ch3G8VL chimeric proteins described above can be co-expressed to form a chimeric antibody, referred to as ch3G8. The chimeric antibody ch3G8 can be expressed either in a myeloma or in other mammalian cells (e.g., CHO, HEK-293). An example of a procedure for expression of CD16A binding proteins such as ch3G8 and variants is provided in Example 4, infra.

### Example 2

### Humanized Anti-CD16A Binding Proteins

A) Humanized Heavy Chain

CDR encoding sequences from the mouse 3G8 V_{H} clone were fused to framework sequences derived from the human germline V_{H} sequence VH2-70 to create a polynucleotide encoding a V_{H} designated Hu3G8VH. The polynucleotide was generated by an overlapping PCR procedure. In a first step, using the primers and strategy shown below and the mouse 3G8 V_{H} polynucleotide (SEQ ID NO: 1) as template.

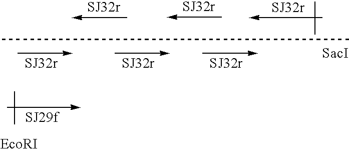

| | | | | | | | | | | | | | | | Seq |
|---|---|---|---|---|---|---|---|---|---|---|---|---|---|---|---|
| | | | | | | | | | | | | | | | ID |
| Primer | Length | Sequence | | | | | | | | | | | | | NO: |
| SJ29f | 62 | ccg | cga | att | ctG | GCC | AGG | TTA | CCC | TGA | GAG | AGT | CTG | GCC | 7 |
| | | CTG | CGC | TGG | TGA | AGC | CCA | GAG | AG | | | | | | |
| SJ30f | 80 | GCG | CTG | GTG | AAG | CCC | ACA | GAG | AGC | CTC | AGA | CTG | ACT | TGT | 8 |
| | | ACC | TTC | TCT | GGG | TTT | TCA | CTG | AGC | ACT | TCT | GGT | ATG | GGT | |
| | | GT | | | | | | | | | | | | | |
| SJ31f | 42 | TGG | ATT | CGT | CAG | CCT | CCC | GGG | AAG | GCT | CTA | GAG | TGG | CTG | 9 |
| | | GCA | | | | | | | | | | | | | |
| SJ32r | 42 | TGC | CAG | CCA | CTC | TAG | AGC | CTT | CCC | GGG | AGG | CTG | ACG | AAT | 10 |
| | | CCA | | | | | | | | | | | | | |
| SJ33f | 72 | GTC | CTC | ACA | ATG | ACC | AAC | ATG | GAC | CCT | GTG | GAT | ACT | GCC | 11 |
| | | ACA | TAC | TAC | TGT | GCT | CGG | ATA | AAC | CCC | GCC | TGG | | | |
| SJ34r | 51 | CAT | GTT | GGT | CAT | TGT | GAG | GAC | TAC | CTG | GTT | TTT | GGA | GGT | 12 |
| | | ATC | CTT | GGA | GAT | | | | | | | | | | |
| SJ35r | 37 | GGC | TGA | GCT | CAC | AGT | GAC | CAG | AGT | CCC | TTG | GCC | CCA | G | 13 |
| SJ37f | 27 | GTG | TAG | GCT | GGA | TTC | GTC | AGC | CTC | CCG | | | | | 14 |
| SJ38r | 33 | GAC | GAA | TCC | AGC | CTA | CAC | CCA | TAC | CAG | AAG | TGC | | | 15 |

The resulting fragment was digested with EcoRI and SacI and cloned into pUC18. After sequencing, one plasmid was selected for a final round of overlapping PCR to correct a deletion which occurred during the second PCR step. The resulting polynucleotide had the sequence:

{hu3G8VH}

SEQ ID NO:16

CAGGTTACCCTGAGAGAGTCTGGCCCTGCGCTGGTGAAGCCCACACAGAC
CCTCACACTGACTTGTACCTTCTCTGGGTTTTCACTGAGCACTTCTGGTA
TGGGTGTAGGCTGGATTCGTCAGCCTCCCGGGAAGGCTCTAGAGTGGCTG
GCACACATTTGGTGGGATGATGACAAGCGCTATAATCCAGCCCTGAAGAG
CCGACTGACAATCTCCAAGGATACCTCCAAAAACCAGGTAGTCCTCACAA
TGACCAACATGGACCCTGTGGATACTGCCACATACTACTGTGCTCGGATA
AACCCCGCCTGGTTTGCTTACTGGGGCCAAGGGACTCTGGTCACTGTGAG
CTCA

The Hu3G8VH sequence was then combined with segments coding for a secretion signal sequence (as described above; lower case underline) and cDNA for the human Cγ1 constant region (lower case). The resulting polynucleotide had the sequence:

{hu3G8VH-1}

SEQ ID NO:17

gctagcgtttaaacttaagcttgttgactagtgagatcacagttctctct
acagttactgagcacacaggacctcaccatgggatggagctgtatcatcc
tcttcttggtagcaacagctacaggtaaggggctcacagtagcaggcttg
aggtctggacatatatatgggtgacaatgacatccactttgcctttctct
ccacaggtgtccactccCAGGTTACCCTGAGAGAGTCTGGCCCTGCGCTG
GTGAAGCCCACACAGACCCTCACACTGACTTGTACCTTCTCTGGGTTTTC
ACTGAGCACTTCTGGTATGGGTGTAGGCTGGATTCGTCAGCCTCCCGGGA
AGGCTCTAGAGTGGCTGGCACACATTTGGTGGGATGATGACAAGCGCTAT
AATCCAGCCCTGAAGAGCCGACTGACAATCTCCAAGGATACCTCCAAAAA
CCAGGTAGTCCTCACAATGACCAACATGGACCCTGTGGATACTGCCACAT
ACTACTGTGCTCGGATAAACCCCGCCTGGTTTGCTTACTGGGGCCAAGGG
ACTCTGGTCACTGTGAGCTCAgcctccaccaagggcccatcggtcttccc
cctggcaccctcctccaagagcacctctgggggcacagcggccctgggct
gcctggtcaaggactacttccccgaaccggtgacggtgtcgtggaactca
ggcgccctgaccagcggcgtgcacaccttcccggctgtcctacagtcctc
aggactctactccctcagcagcgtggtgaccgtgccctccagcagcttgg
gcacccagacctacatctgcaacgtgaatcacaagcccagcaacaccaag
gtggacaagagagttgagcccaaatcttgtgacaaaactcacacatgccc
accgtgcccagcacctgaactcctggggggaccgtcagtcttcctcttcc
ccccaaaacccaaggacaccctcatgatctcccggacccctgaggtcaca
tgcgtggtggtggacgtgagccacgaagaccctgaggtcaagttcaactg
gtacgtggacggcgtggaggtgcataatgccaagacaaagccgcgggagg
agcagtacaacagcacgtaccgtgtggtcagcgtcctcaccgtcctgcac
caggactggctgaatggcaaggagtacaagtgcaaggtctccaacaaagc
cctcccagcccccatcgagaaaaccatctccaaagccaaagggcagcccc
gagaaccacaggtgtacaccctgcccccatcccgggatgagctgaccaag
aaccaggtcagcctgacctgcctggtcaaaggcttctatcccagcgacat
cgccgtggagtgggagagcaatgggcagccggagaacaactacaagacca
cgcctcccgtgctggactccgacggctccttcttcctctacagcaagctc
accgtggacaagagcaggtggcagcaggggaacgtcttctcatgctccgt
gatgcatgaggctctgcacaaccactacacgcagaagagcctctccctgt
ctccgggtaaatgagtgcggccgcgaattc

For expression in mammalian cells (HEK-293), the Hu3G8VH-1 sequence was cloned into the pCI-Neo polylinker at the NheI-EcoRI sites, following intervening cloning into pUC and pCDNA3.1.

B) Humanized Light Chain

CDR encoding sequences from the mouse 3G8 V_{L} clone were fused to framework sequences derived from the human B3 germline V- gene. The polynucleotide was generated by an overlapping PCR procedure using the primers and strategy shown below and the mouse 3G8 V_{L} polynucleotide (SEQ ID NO: 2) as template.

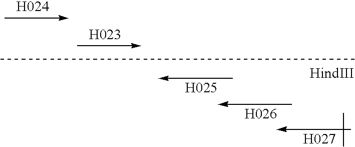

| | | | SEQ |
|---|---|---|---|
| | | | ID |
| Primer | Length | Sequence | NO: |
| H023 | 63 | ACTCTTTGGCTGTGTCTCTAGGGGAGAGGGCCACCATCAACTGCA | 18 |
| | | A | |
| | | GCCAGCCAAAGTGTTG | |
| H024 | 66 | CTCTCCACAGGTGTCCACTCCGACATCGTGATGACCCAATCTCCA | 19 |
| | | G | |
| | | ACTCTTTGGCTGTGTCTCTA | |
| H025 | 71 | GGTGAGGGTGAAGTCTGTCCCAGACCCACTGCCACTAAACCTGTC | 20 |
| | | T | |
| | | GGGACCCCAGATTCTAGATTGGATG | |
| H026 | 67 | TGACAGTAATAAACTGCCACATCCTCAGCCTGCAGGCTGCTGATG | 21 |
| | | G | |
| | | TGAGGGTGAAGTCTGTCCCAG | |
| H027 | 71 | gcggcAAGCTTGGTCCCCTGTCCGAACGTGTACGGATCCTCATTA | 22 |
| | | C | |
| | | TTTGCTGACAGTAATAAACTGCCAC | |
| H009 | 30 | CGAGCTAGCTGAGATCACAGTTCTCTCTAC | 23 |

The resulting polynucleotide had the sequence:

{hu3G8VL}

SEQ ID NO:25

GACACTGTGCTGACCCAATCTCCAGCTTCTTTGGCTGTGTCTCTAGGGCA
GAGGGCCACCATCTCCTGCAAGGCCAGCCAAAGTGTTGATTTTGATGGTG
ATAGTTTTATGAACTGGTACCAACAGAAACCAGGACAGCCACCCAAACTC
CTCATCTATACTACATCCAATCTAGAATCTGGGATCCCAGCCAGGTTTAG
TGCCAGTGGGTCTGGGACAGACTTCACCCTCAACATCCATCCTGTGGAGG
AGGAGGATACTGCAACCTATTACTGTCAGCAAAGTAATGAGGATCCGTAC
ACGTTCGGAGGGGGGACCAAGCTTGAGATCAAA

The Hu3G8 V_{L} gene segment was combined with a signal sequence (as described above, lower case, underline) and a human C- constant region (lower case) cDNA using standard techniques resulting in a product with the sequence below:

{hu3G8VL-1}

SEQ ID NO:26

gctagctgagatcacagttctctctacagttactgagcacacaggacctc
accatgggatggagctgtatcatcctcttcttggtagcaacagctacagg
taaggggctcacagtagcaggcttgaggtctggacatatatatgggtgac
aatgacatccactttgcctttctctccacaggtgtccactccGACACTGT
GCTGACCCAATCTCCAGCTTCTTTGGCTGTGTCTCTAGGGCAGAGGGCCA
CCATCTCCTGCAAGGCCAGCCAAAGTGTTGATTTTGATGGTGATAGTTTT
ATGAACTGGTACCAACAGAAACCAGGACAGCCACCCAAACTCCTCATCTA
TACTACATCCAATCTAGAATCTGGGATCCCAGCCAGGTTTAGTGCCAGTG
GGTCTGGGACAGACTTCACCCTCAACATCCATCCTGTGGAGGAGGAGGAT
ACTGCAACCTATTACTGTCAGCAAAGTAATGAGGATCCGTACACGTTCGG
AGGGGGGACCAAGCTTGAGATCAAACGAACTGTGGCTGCACCATCGGTCT
TCATCTTCCCGCCATCTGATGAGCAGTTGAAATCTGGAACTGCCTCTGTT
GTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGC
AGGACAGCAAGGACAGCACCTACAGCCTCAGCAGCACCCTGACGCTGAGC
AAAGCAGACTACGAGAAACACAAAGTCTACGCCTGCGAAGTCACCCATCA
GGGCCTGAGCTCGCCCGTCACAAAGAGCTTCAACAGGGGAGAGTGTTAGT
TCTAGAGTCGACTCTAGAGGATCCCCGGGTACCGAGCTCGAATTC

This construct was inserted into pCI-Neo for expression in mammalian cells.

### Example 3

### Variant CD16A Binding Proteins

Additional expression constructs were made in which sequence changes were introduced in the V_{L} or V_{H} domains by site directed mutagenesis. A typical mutagenesis reaction contained 10 ng plasmid DNA (isolated from a methylation competent strain of E. coli), 125 ng each of a forward and reverse primer, each containing the mutation of interest, reaction buffer, and dNTPs in 0.05 ml volume. 2.5 units of PFUTURBO® DNA polymerase (Stratagene) was added and the reaction was subjected to 15 cycles of 95°, 30 sec; 55°, 1 min; 68°, 12 min. The product of the PCR was then digested with DpnI endonuclease and the restricted DNA was used to transform E. coli, strain XL-10 GOLD® ultracompetent cells. Because DpnI only digests methylated DNA it will digest the parental, non-mutated, plasmid leaving the newly synthesized non-methylated product, containing the mutation of interest, as the predominant species.

The sequences of the variant V_{H} domains are shown in Table 4. The sequences of the variant V_{L} domains are shown in Table 5.

### Example 4

### Expression in Mammalian Cells

Various combinations of heavy and light chain expression plasmids (e.g., comprising the chimeric, humanized and variant V_{L} and V_{H} domains fused to human Cγ1 and C constant domains as described above) were co-transfected into HEK-293 cells for transient expression of recombinant tetrameric antibodies (i.e., comprising 2 heavy chains and 2 light chains), sometimes referred to herein as “recombinant antibodies.” Transfection was carried out using LIPOFECTAMINE® 2000 (Invitrogen) in 6 well plates according to the manufacturer's instructions.

Recombinant antibodies were prepared by cotransfection of a heavy chain expression plasmid (i.e., encoding a heavy chain comprising a V_{H} and constant domains) and light chain expression plasmids (i.e., encoding a light chain comprising a V_{L} and constant domains) together into HEK-293 cells for transient expression of recombinant antibodies.

Hu3G8VH variants listed in Table 4 were coexpressed with the hu3G8VL-1 light chain. For reference, most assays included (i) recombinant antibodies produced by coexpression of ch3G8VH and ch3G8VL (“ch3G8VH/ch3G8VL”) and (ii) recombinant antibodies produced by coexpression of hu3G8VH-1 and hu3G8VL-1 (“hu3G8VH-1/hu3G8VL-1”).

Hu3G8VL variants listed in Table 5 were coexpressed with the ch3G8VH heavy chain. For reference, most assays included (i) recombinant antibodies produced by coexpression of ch3G8VH and ch3G8VL (“ch3G8VH/ch3G8VL”) and (ii) recombinant antibodies produced by coexpression of ch3G8VH and hu3G8VL-1 (“ch3G8VH/hu3G8VL-1”).

After three days, the levels of recombinant antibodies in the conditioned media were determined by ELISA, and the recombinant antibodies were analyzed by ELISA for binding to captured sCD16A as described in Examples 5. Selected antibodies were assayed for cell binding to cells expressing the extracellular domain of CD16A, as shown in Example 6.

### Example 5

### ELISA Determination of Binding to CD16A

Sandwich ELISA was performed to detect binding of antibodies to a soluble form of CD16A.

Soluble Human CD16A

A soluble form of human CD16A was expressed from HEK-293 cells using a pcDNA3.1-derived expression vector containing the CD16A gene truncated just prior to the transmembrane region. To create the vector, cDNA encoding CD16A was amplified using the primers 3A_{left} [gttggatcctccaactgctctgctacttctagttt] (SEQ ID NO:27) and 3A_{right} [gaaaagcttaaagaatgatgagatggttgacact] (SEQ ID NO:28) digested with BamHI and HindIII, and cloned into the vector pcDNA3.1 (Novagen) at the Bam/HindIII site of the polylinker. The construct was used to transiently transfect HEK-293 cells. For some assays, the secreted product was purified from conditioned medium using affinity chromatography on a human IgG Sepharose column. In some assays, the amount of sCD16A in conditioned medium was quantitated and unpurified sCD16A was used. Purification was not required since the ELISA capture antibody (LNK16 mAb) specifically bound the antigen, allowing removal of contaminants in washing steps.

The amino acid sequence of the sCD16A construct is shown below. (The signal sequence, underlined, is cleaved off during expression. Note the last seven residues are derived from the vector pCDNA3.1 rather than from the CD16A gene):

(SEQ ID NO:29)

MWQLLLPTALLLLVSAGMRTEDLPKAVVFLEPQWYRVLEKDSVTLKCQGA
YSPEDNSTQWFHNESLISSQASSYFIDAATVDDSGEYRCQTNLSTLSDPV
QLEVHTGWLLLQAPRWVEKEEDPIHLRCHSWKNTALHKVTYLQNGKGRKY
FHHNSDFYIPKATLKDSGSYFCRGLFGSKNVSSETVNITITQGLAVSTIS
SETKLAAARV

ELISA Format

Plates were first coated with 100 ng/well of the anti-CD16A mAb LNK-16 (Advanced ImmunoChemical, Long Beach Calif.; see 5th Human Lymphocyte Differentiation Antigens Workshop) in carbonate buffer at room temperature for 2 hrs. Any anti-sCD16A antibody that does not block binding by 3G8 can be used. After blocking for 30 minutes with PBS-T-BSA, sCD16A conditioned medium was added at a dilution of 1/10 and incubated at room temperature for 16 hrs. Alternatively, when purified sCD16A was used, it was diluted to a concentration of 50 ng/ml in PBS-T-BSA. 0.05 ml was added to each well and incubated for at least 2 hrs at room temperature.

The plate was washed and dilutions of recombinant antibodies starting at 0.5 μg/ml in PBS-T-BSA were then added and incubated for 1 hr at room temp. Binding of recombinant antibodies to the captured sCD16A was then measured using an anti-human IgG-HRP conjugate and TMB substrate. After stopping color development using dilute sulfuric acid, the plate was read at 450 nM.

Results of Binding Assays

This example shows that the binding properties of humanized anti-CD16A antibodies for binding to CD16A are the same or similar to the properties of the chimeric 3G8 antibody.

Based on the comparative binding studies, the recombinant antibodies were classified as binding with high, intermediate, or low affinity. Antibodies with high and intermediate binding affinity are discussed above in section 4. The recombinant antibodies with a V_{H} domain of hu3G8VH- 9, 10, 11, 13, 15, 21, 38, 39, or 41 showed little or no binding to sCD16A. From these data it appears certain substitutions (or combinations of substitutions) are generally detrimental to binding. For example, substitution of tyrosine or aspartic acid at V_{H} position 52 (i.e., 52Y and 52D) or threonine at position 94 (94T) are detrimental to binding. Similarly, the combination of leucine at position 50 with aspartic acid at position 54 (50L+54N) is detrimental to binding, as is the combination of arginine at 94 and aspartic acid at 101 (94R+101 D). However, aspartic acid at 101 is tolerated when position 94 is glutamine, lysine, histidine or alanine (but not arginine). Further 34V+94R+101D has intermediate activity. This indicates a relationship between positions 34, 94 and 101 in maintaining high affinity binding, and suggests that 34V may be an especially important residue. Likewise, recombinant antibodies with a V_{L} domain of hu3G8VL-6, 7, 8, 9, 11, 12, 13, and 14 showed little or no binding to sCD16A. From these data it appears certain substitutions (or combinations of substitutions) are generally detrimental to binding. For example, substitution of alanine at position 34 (34A) or tyrosine at position 92 (92Y) is generally detrimental to binding.

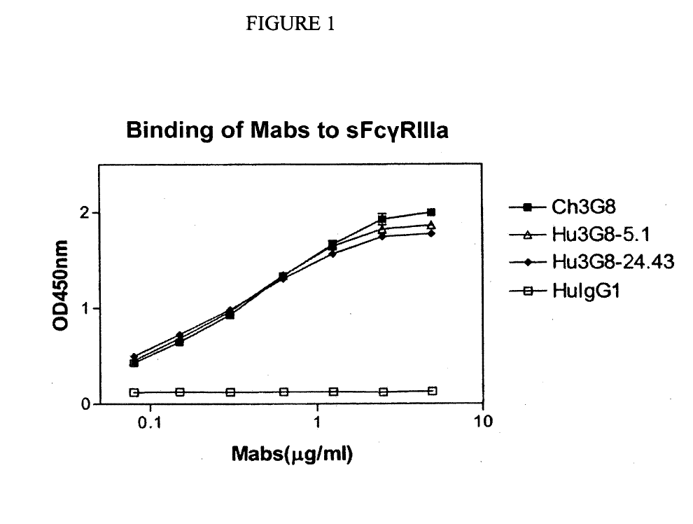

Results of an exemplary binding assay are shown in FIG. 1.

### Example 6

### Antibody Binding to Cells Expressing CD16A

The ability of selected humanized antibodies to bind to CD16A expressed by CHO-K1 cells as assayed by direct binding competition assays.

CHO-K1 cells expressing extracellular domain of FcγRIIIA fused to the transmembrane and intracellular domain of FcγRIIb were used for cell binding assays. Cells were plated at 40,000 cells per well in 96 well flat bottom tissue culture plates (FALCON® MICROTEST™ Tissue Culture plate, 96 well) and incubated at 37° C. CO₂ incubator for approximately 24 hr. The plate was then gently washed three times with 25 mM Hepes, 75 uM EDTA, 11.5 mM KCI, 115 mM NaCl, 6 mM MgSO4, 1.8 mM CaCl2, 0.25% BSA (binding buffer).

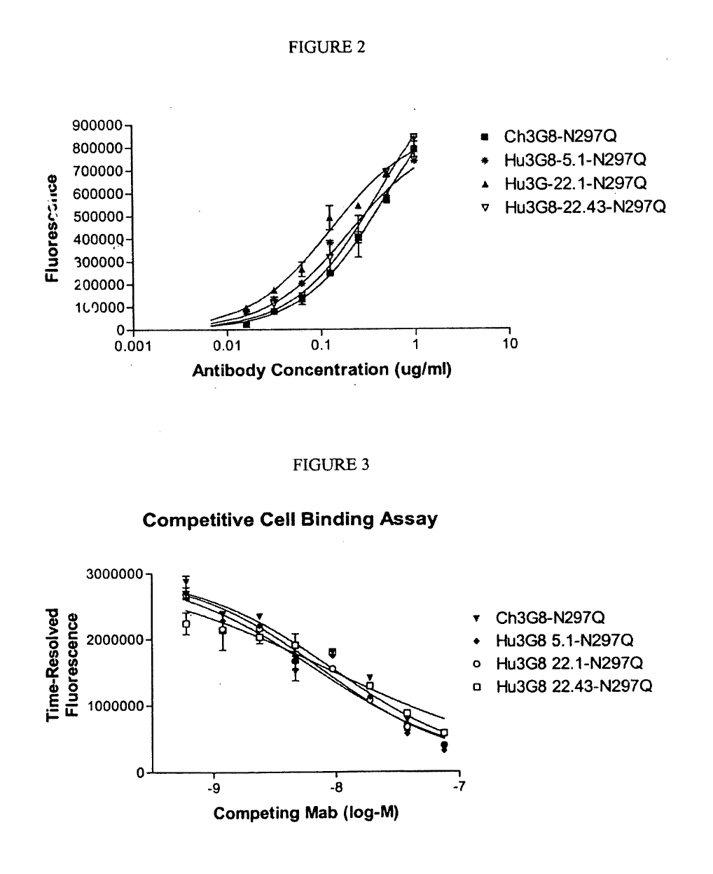

For indirect binding assays, 100 μl of a serial dilution of anti-CD16A mAb (final concentration: 1 ug/ml, 0.5, 0.25, 0.125, 0.0625, 0.03, 0.015, 0 ug/ml) was then added to wells in binding buffer. The plate was then incubated at 23° C. for 1 hr and washed three times with binding buffer. 50 μl/well of Europium (EU)-labeled-antihuman-IgG (100 ng/ml) was then added to each well and the plate was incubated at 23° C. for 30 minutes then washed three times with binding buffer. Finally, 100 μl DELFIA® enhancement solution, an acidic chelating detergent solution intended for use in the quantitative determination of Eu³⁺/Sm³⁺ (PerkinElmer/Wallac) was added. After incubating with shaking for 15 minutes, the plate was read for time resolved fluorescence (excitation 340 nm; emission 615 nm) in a VICTOR² instrument (PerkinElmer/Wallac). The results of the assay are shown in FIG. 2.

The CHO-K1 cells described above were also used in competition assays. After washing with binding buffer as described above, varying amounts of purified unlabeled mAb (1.2-75 nM final concentration) were mixed with a fixed concentration of Eu-Ch3G8-N297Q (final concentration 2.5 nM). The plate was then incubated at 23° C. for 1 hr and washed three times with binding buffer. 100 μl DELFIA® enhancement solution (PerkinElmer/Wallac) was the added and after incubating with shaking for 15 minutes, the plate was read for time resolved fluorescence (excitation 340 nm; emission 615 nm) in a VICTOR² instrument (PerkinElmer/Wallac). The results of the assay are shown in FIG. 3.

These assays demonstrate that the humanized anti CD16A monoclonal antibodies bind with high affinity to CD16A on the surface of transfected cells. Hu3G8-22.1-N297Q binds to CD16A bearing cells with higher affinity than Ch3G8-N297Q.

### Example 7

### Inhibition of Binding of sCD16A to Immune Complexes

Assay of 4-4-20 Binding to FITC-BSA

The binding of ch4-4-20 or ch4-4-20 (D265A) to FITC-BSA was assessed by ELISA. (Ch4-4-20 is identical to Ch3G8 except that it contains the respective V_{H} and V_{L} regions of 4-4-20 instead of those of 3G8. Thus it retains high affinity and specificity for the hapten fluorescein. 4-4-20 is described in Bedzyk et al., 1989, J. Biol. Chem. 264:1565-9.) FITC-BSA (1 ug/ml-50 ng/well) was coated onto Nunc maxisorb immunoplates in carbonate buffer and allowed to bind for approximately 16 hr. Following blocking with BSA, dilutions of ch4-4-20 were added to the wells and allowed to bind for 1 hr at RT. After washing out unbound mAb, HRP-conjugated goat anti-human Ig secondary was added. One hour later the secondary antibody was removed, washed and developed with TMB substrate. Following addition of an acidic stop solution the plate was read at 450 nm. Both ch4-4-20 and ch4-4-20(D265A) bound to the FITC-BSA with high affinity (data not shown).

Assay of sFcR binding to ch4-4-20/FITC-BSA immune complexes

The same format was used to assay binding of sFcRs to immune complexes (IC) formed on the ELISA plate between ch4-4-20 and FITC-BSA. In this case we have used either biotinylated sFcR or biotinylated anti-human G2 mAb as a secondary reagent, followed by streptavidin-HRP detection.

Inhibition of sFcR binding to IC by murine, chimeric and humanized 3G8

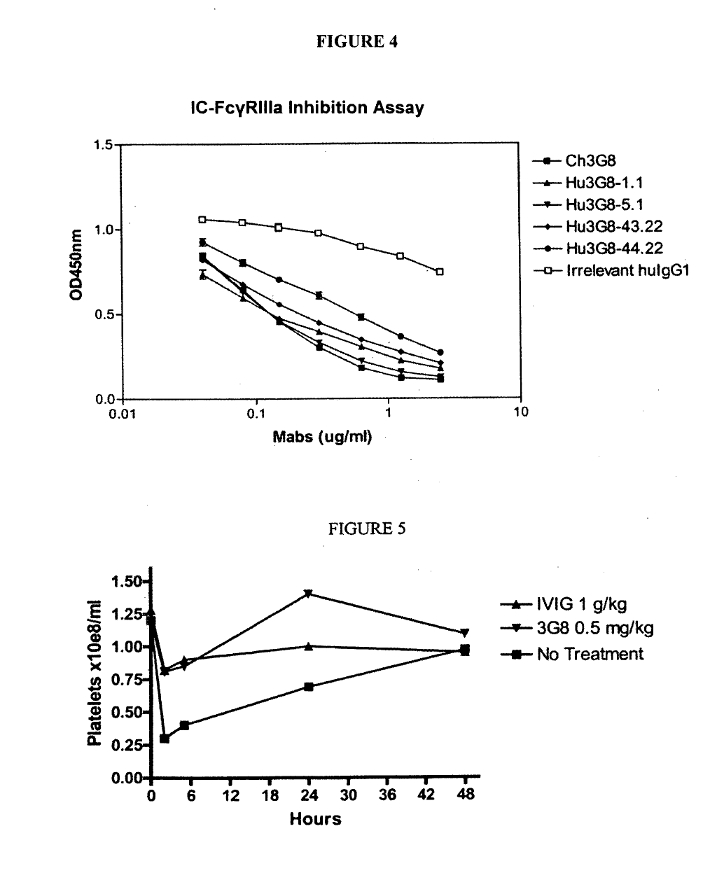

The concentrations of ch4-4-20 and sFcR were fixed to give approximately 90 percent maximal signal in the assay. sCD16A was premixed with serial dilutions of murine, chimeric or humanized 3G8 and incubated for one hour prior to adding to the plate containing the immune complex. Serial dilutions of humanized or chimeric 3G8 were incubated with sCD16A-G2-biotin for one hour. The mixtures were then added to ELISA wells containing an immune complex between a human IgG1 chimeric form of the anti-fluorescein mAb 4-4-20 and FITC-BSA. After one hour, binding of the soluble receptor to the IC was detected using streptavidin-HRP conjugate and TMB development. The results are shown in FIG. 4. This assay indicates that humanized anti-CD16A antibodies are potent inhibitors of CD16A binding to IgG in immune complexes.

### Example 8

### Analysis of Anti-CD16A Monoclonal Antibody Panel

A panel of hybridomas was generated following immunizing and boosting mice with sCD16A using standard methods. Eight 96-well plates were screened by ELISA for binding activity on plates coated directly with sCD16A. Ninety-three of these gave a positive signal and were expanded further. Of these, 37 were positive for binding to human blood cells by FRCS. These supernatants were then analyzed for their ability to block the interaction of CD16A with immune complexes and for the similarity of the binding site (epitope) to that of 3G8. Assays included capture ELISA using chimeric 3G8 down and inhibition of immune complex binding to sRIIIa-Ig. Based on these assays antibodies with binding and inhibitory properties similar to 3G8 were isolated, as well as mAbs with binding and/or inhibitory properties distinct from 3G8.

DJ130c (DAKO) and 3G8 were used as controls in the assays. mAb DJ130c is a commercially available mAb which binds CD16 at an epitope distinct from 3G8 (Tatum and Schmidt). This mAb does not block FcγRIIIa-immune complex binding (Tamm and Schmidt). In an ELISA-based inhibition assay, DJ130c enhances rather than inhibits binding.

The data indicate that the panel contains antibodies which bind to the same epitope as Ch3G8 and block sCD16A binding to immune complexes (Table 3). The panel of mAbs also contains antibodies which do not bind to the same epitope as Ch3G8. Most of these latter antibodies do not block the interaction of sCD16a with IgG in immune complexes.

TABLE 3

| | Effect on sCD16a Binding to Immune Complexes | | | |
|---|---|---|---|---|
| Assay | Result1 | Inhibition | Enhancement | No Effect |
| Binding to | Positive | 2 | 5 (+DJ-130c) | 17 |
| sCD16 | Negative | 11 (+3G8) | 0 | 2 |
| Captured by | | | | |
| Ch3G8 | | | | |

### Example 9

### Induction of Platelet Depletion In Vivo

The in vivo activity of a CD16A binding protein for blocking human Fc-FcγRIII interactions induced by autoantibodies can be evaluated using animal models of autoimmune diseases. One suitable model is the “passive mouse model” of ITP and the anti-platelet mAb 6A6 (see, Oyaizu et al., 1988, J. Exp. Med. 167:2017-22; Mizutani et al., 1993, Blood 82:837-44). 6A6 is an IgG2a isotype mAb derived from a NZW×BSXB F1 individual. Administration of 6A6 depletes platelets in muFcγRIII −/−, huFcγRIIIA transgenic mice but not in muFcγRIII −/− mice without the human transgene. See Samuelsson et al., 2001, Science 291:484-86. Other anti-platelet monoclonal antibodies can be used in place of 6A6 in the model. Alternatively, a polyclonal anti-platelet antibody can be used.

CD16A binding proteins that confer the greatest degree of protection from platelet depletion can be identified by administrating CD16A binding proteins to a muFcγRIII −/−, huFcγRIIIA transgenic mouse and measuring any reduction in mAb 6A6 induced platelet depletion.

A related assay can be carried out using a chimeric human IgGIK chimeric derivative of 6A6 in place of the mouse mAb in the protocol provided above, so that the depleting mAb had a human isotype. To conduct this assay, a chimeric 6A6 monoclonal antibody (ch6A6) was prepared by fusing the cDNA segments encoding the murine anti-platelet monoclonal antibody 6A6 V_{H} and V_{L} regions to the human Cγ1 and C cDNA segments, respectively. The resulting genes were co-expressed in 293 cells and chimeric 6A6 was purified by protein A affinity chromatography followed by size exclusion chromatography.

To demonstrate that the chimeric 6A6 antibody induces platelet depletion, to and ch6A6 was administered to muFcγRIII^{-/-}, huFcγRIIIA transgenic mice. The ch6A6 was administered to each animal either i.v. or intraperitoneally (i.p.) (0.1 μg/g). Animals were bled 2 hrs, 5 hrs, 24 hrs and 48 hrs after administration of ch6A6, and plasma platelet counts were determined using a Z2™ COULTER COUNTER® particle counter and size analyzer equipped with a 70 μm aperture. Particles between 1.5 and 4 μm in size (corresponding to platelets) were counted and the data were analyzed by plotting the platelet count versus time for each concentration.

Two hours after injection of 0.1 μg/g ch6A6 i.p., approximately 75% of the platelets were depleted. The number of platelets remained low for 5 hours after ch6A6 injection then progressively increased to return to normal 72 hours after ch6A6 injection.

Two hours after injection of 0.1 μg/g ch6A6 i.v., approximately 60% of the platelets were depleted. The number of platelets remained low for 6 hours after ch6A6 injection then progressively increased to return to normal 48 hours after ch6A6 injection.

### Example 10

### Analysis of the Ability of CD16A Binding Antibodies to Protect Mice from Platelet Depletion

The ability of CD16A binding proteins to reduce platelet depletion in experimental ITP can be assayed as described below. CD16A binding proteins were administered intravenously (i.v.) to groups of muFcγRIII^{-/-}, huFcγRIIIA transgenic mice at concentrations of 0.5, 1, 2 or 5 μg/g in phosphate buffered saline (PBS). Controls were PBS alone or an irrelevant human IgGI (negative control) or human intravenous immunoglobulin (IVIG; positive control). One hour after administration of the CD16A binding protein or control, ITP was induced by administering 0.1 μg/g ch6A6 to each animal either intravenously or intraperitoneally. Animals were bled 2 hrs, 5 hrs, 24 hrs and 48 hrs after administration of ch6A6. Plasma platelet counts were determined using the Z2™ COULTER COUNTER® particle counter and size analyzer as described above and the data were analyzed by plotting the platelet count versus time for each concentration of administered binding protein.

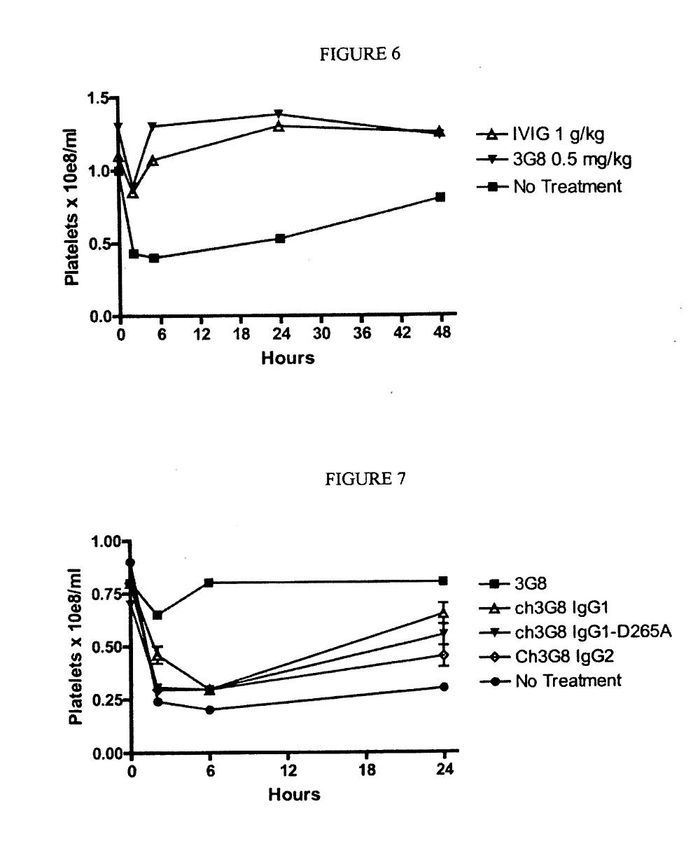

When muFcγRIII^{-/-} huFcγRIIIA transgenic mice were injected with murine 3G8 (0.5 μg/g) one hour before i.p. injection of ch6A6, 33% of the platelets were depleted at the 2 hours time point (FIG. 5). The number of platelets then progressively increased to return to normal 24 hours after ch6A6 injection. When muFcγRIII^{-/-}, huFcγRIIIA transgenic mice were injected with murine 3G8 (0.5 μg/g) one hour before i.v. injection of ch6A6, 30% of the platelets were depleted at the 2 hours time point (FIG. 6). The number of platelets then rapidly increased to return to normal 5 hours after ch6A6 injection.

These results were similar to the protection seen when human IVIG is administered. When muFcγRIII^{-/-}, huFcγRIIIA transgenic mice were injected with human IVIG (1 mg/g) one hour before i.p. injection of ch6A6, 33% of the platelets were depleted at the 2 hours time point (FIG. 5). The number of platelets then progressively increased to return to normal 24 hours after ch6A6 injection. When muFcγRIII^{-/-}, huFcγRIIIA transgenic mice were injected with human IVIG (1 mg/g) one hour before i.v. injection of ch6A6, 20% of the platelets were depleted at the 2 hours time point (FIG. 6). The number of platelets then rapidly increased to return to normal 5 hours after ch6A6 injection.

The results shown in FIGS. 5 and 6 show that m3G8 protects mice from ch6A6-mediated platelet depletion, and that the level of protection was similar to the protection conferred by IVIG.

Preparations of recombinant mouse 3G8 produced in HEK-293 cells, chimeric 3G8 with human IgG1 or IgG2 constant domains (ch3G8-γ1 produced in HEK-293 and CHO-K1 cells, and ch3G8-γ2 produced in HEK-293 cells), and a ch3G8-γ1 variant (ch3G8-γ1 D265A) did not provide significant protection in this experiment. When muFcγRIII^{-/-}, huFcγRIIIA transgenic mice were injected with ch3G8° C.1 or γ2 (0.5 μg/g) one hour before i.p. injection of 6A6, approximately 60% of the platelets were depleted at the 5 hour time point (FIG. 7). The number of platelets then progressively returned to normal. Although depletion was not as severe as in mice that received no anti-CD16A binding protein, these chimeric antibodies provided significantly less protection, if any, than murine 3G8. A ch3G8 variant in which aspartic acid 265 was changed to alanine showed similar results. Interestingly, as is shown in Example 11, modification of the ch3G8 to produce an aglycosylated variant increased the protective effect of the antibody.

### Example 11

### Ch3G8 N297Q Protects Mice from ch6A6-Mediated Platelet Depletion

An aglycosylated version of ch3G8-γ1 was prepared by mutating the expression polynucleotide encoding ch3G8-γ1 so that residue 297 was changed from asparagine (N) to glutamine acid (Q), and expressing the encoded antibody. Residue 297 lies in an N-linked glycosylation site, and this mutation prevents glycosylation of the Fc domain at this site. This aglycosylated antibody, ch3G8 N297Q, was produced in HEK-293 cells as described for ch3G8-γ1 (see Example 4, supra). The ability of ch3G8-N297Q to protect against ch6A6-mediated platelet depletion was tested using the protocol described above.

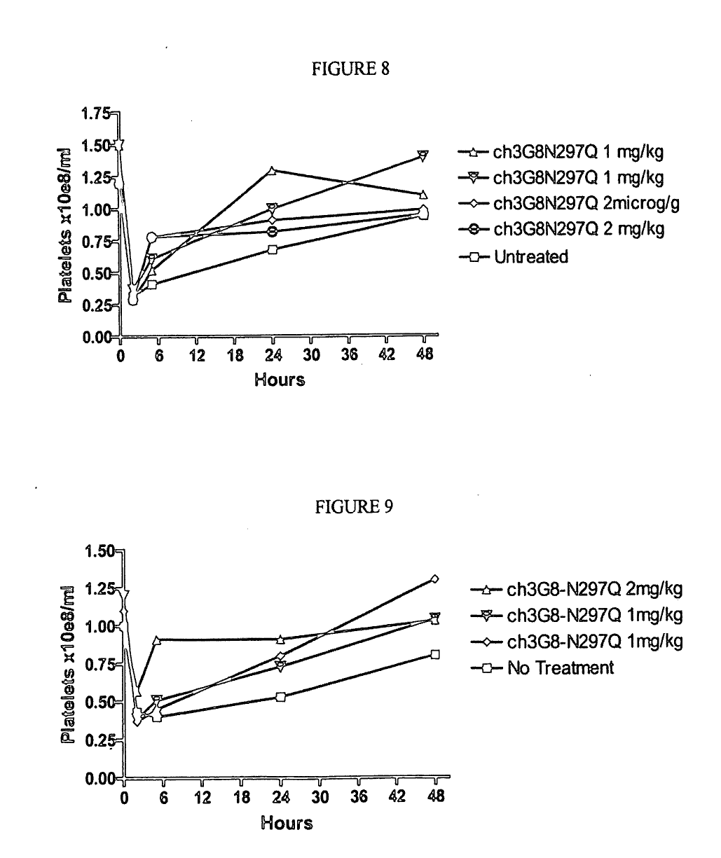

When muFcγRIII^{-/-}, huFcγRIIIA transgenic mice were injected with 1 μg/g of the aglycosyl form of ch3G8 (ch3G8 N297Q) one hour before i.p. injection of ch6A6, approximately 75% of the platelets were depleted at the 2-hour time point (FIG. 8). Platelet levels increased faster than in the absence of ch3G8 N297Q, and returned to normal by 24 hours after ch6A6 injection.

When muFcγRIII^{-/-} huFcγRIIIA transgenic mice were injected with 1 μg/g ch3G8 N297Q one hour before i.v. injection of ch6A6, approximately 60% of the platelets were depleted at the 2 hours time point (FIG. 9). Platelet levels increased faster than in the absence of ch3G8 N297Q, and returned to normal by 48 hours after ch6A6 injection.

When muFcγRIII^{-/-}, huFcγRIIIA transgenic mice were injected with ch3G8 N297Q (2 μg/g) one hour before i.v. injection of ch6A6, only 40% of the platelets were depleted at the 2 hours time point (FIG. 9). Platelet levels increased faster than in the absence of ch3G8 N297Q, and returned to normal by 5 hours after ch6A6 injection.

Thus, ch3G8-N297Q was consistently able to significantly improve platelet counts. Binding of 3G8 to human CD16A on effector cells blocks the ability of CD16A to interact with immune complexes and trigger effector functions such as ADCC or phagocytosis. Chimeric and mouse 3G8 molecules have similar ability to bind CD16A and are similar in their ability to inhibit the binding of sCD16A to immune complexes in vitro. Without intending to be bound by a particular mechanism, the binding (and thus) the blocking activity of the mAb is thought to be confined to the Fab portion of the antibody and blocking of huCD16A is believed to be the mechanism of protection in the transgenic mouse ITP model. The data above suggest that the glycosylation state of the Fc domain can affect the in vivo protective capacity of anti-CD16A antibodies. Ablation of Fc domain glycosylation (e.g., with D265A or N297Q mutations, or by using a human gamma2 Fc domain) reduces or eliminates Fc binding to FcR. In the case of the aglycosyl (N297Q) variant, complement fixation is also abolished.

### Example 12

### Neutrophil Levels Following Administration of Aglycosyl CD16A Binding Proteins

The effect of an aglycosylated CD16A binding protein on neutrophil levels was tested and compared to that of glycosylated CD16A binding proteins. CD16A binding proteins, or the controls such as irrelevant human IgG1 (negative control) or murine RB6-8C5 (positive control), were administered to groups of muFcγRIII^{-/-}, huFcγRIIIB transgenic mice at a concentration of 5 μg/g in phosphate buffered saline (PBS). Another negative control was administered PBS alone. Twenty four hours later, mice were euthanized and blood, spleen and bone marrow were collected. Neutrophils were analyzed by FACS. Staining experiments were performed in RPMI containing 3% FCS. Murine cells were stained using FITC-conjugated 3G8 (PharMingen) and R-PE-conjugated RB6-8C5 (PharMingen). Samples were analyzed by flow-cytometry using a FACSCALIBUR™ (Becton Dickinson).

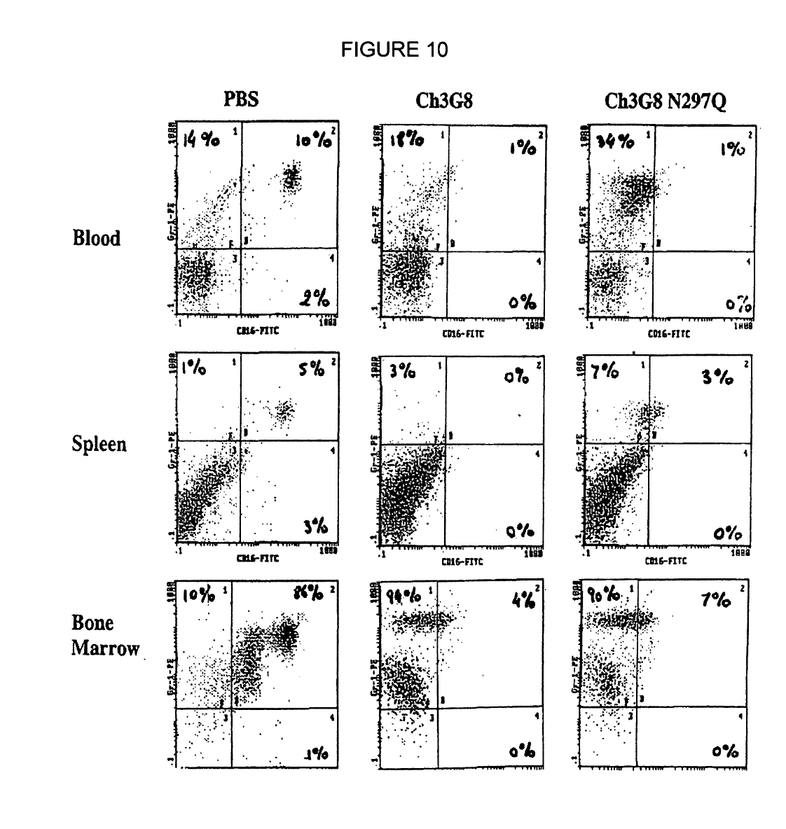

Intraperitoneal injection of 5 μg/g ch3G8 (prepared as described above) resulted in murine neutrophil depletion in the blood and spleen (FIG. 10; upper right quadrant). Similar results were seen following administration of murine 3G8 (results not shown). In the bone marrow of ch3G8 treated animals, neutrophils stained weakly for CD16, which could indicate receptor occupancy by the chimeric antibody or shedding (FIG. 10; see shift from the upper right quadrant to the upper left quadrant). In contrast, intraperitoneal injection of 5 μg/g ch3G8 N297Q did not result in murine neutrophil depletion in the blood, spleen or bone marrow (FIG. 10). In additional experiments, humanized glycosylated 3G8 antibodies showed substantially more depletion of circulating blood neutrophils compared to aglycosylated forms of the same antibodies.

### Example 13

### Autoimmune Hemolytic Anemia Model

This example demonstrates that administration of CD16A binding protein prevents red blood cell depletion in a model of autoimmune hemolytic anemia.

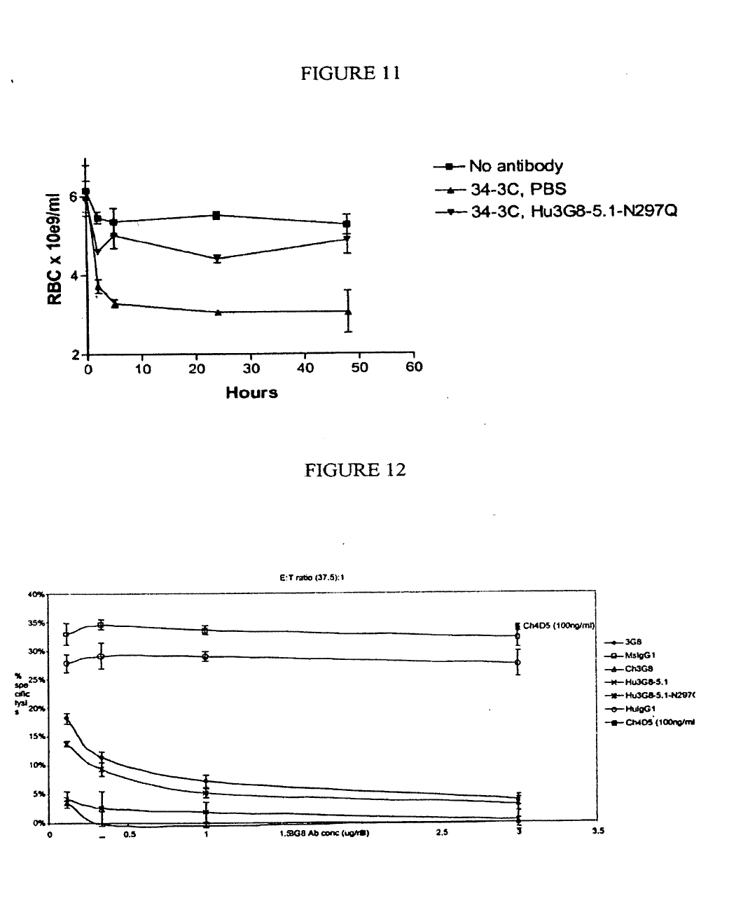

The ability of the Hu3G8-5.1-N297Q monoclonal antibody to prevent antibody-dependent red blood cell depletion in muFcγRIII−/−, huFcγRIIIa+ mice was evaluated. Hu3G8-5.1-N297Q is an aglycosy antibody with the heavy chain Hu3G8VH-5 and the light chain Hu3G8VH-1 and the indicated substitution of asparagine 297. Mice were bled on day 0 and RBC levels were determined using a Z2™ COULTER COUNTER® particle analyzer. The next day groups of 3 animals each were then injected intravenously with either 0.5 mg/kg Hu3G8-5.1-N297Q or PBS. One group of mice did not receive any compound. One hour later, RBC depletion was induced in the first two groups by administering mouse anti-RBC IgG2a mAb 34-3C to each animal intraperitoneally (i.p.) (2.5 mg/kg). Animals were bled 2 hrs, 5 hrs, 24 hrs and 48 hrs after administration of 34-3C and RBC counts were determined. Data was analyzed by plotting RBC count versus. The data, depicted in FIG. 11, demonstrate the ability of Hu3G8-5.1-N297Q to prevent RBC depletion in this model.

### Example 14

### Inhibition of Antibody-Dependent Cellular Cytotoxicity (ADCC)

This example demonstrates that humanized 3G8 variants inhibit ADCC in vitro and with an activity similar to that of mouse 3G8.

Methods: The protocol for assessment of antibody-dependent cell-mediated cytotoxicity (ADCC) is similar to that previously described in (Ding et al., 1998, Immunity 8:403-11). Briefly, target cells from the HER2-overexpressing breast cancer cell line SK-BR-3 were labeled with the europium chelate bis(acetoxymethyl) 2,2′:6′,2″-terpyridine-6,6″-dicarboxylate (DELFIA® BATDA Reagent, Perkin Elmer/Wallac). The labeled target cells were then opsonized (coated) with either chimeric anti-HER2 (ch4D5, 100 ng/ml) or chimeric anti-fluorescein (ch4-4-20, 1 ug/ml) antibodies. In the case of the anti-fluorescein antibody, SK-BR-3 cells were coated with the fluorescein hapten prior to antibody opsonization. Peripheral blood mononuclear cells (PBMC), isolated by FICOLL-PAQUE™ (Amersham Pharmacia) gradient centrifugation, were used as effector cells (Effector: Target ratio:ch4D5=(37.5:1) and ch4-4-20=(75:1)). Following a 3.5 hour incubation at 37° C., 5% CO2, cell supernatants were harvested and added to an acidic europium solution (DELFIA® Europium Solution, Perkin Elmer/Wallac). The fluorescence of the Europium-TDA chelates formed was quantitated in a time-resolved fluorometer (VICTOR² 1420, Perkin Elmer/Wallac). Maximal release (MR) and spontaneous release (SR) were determined by incubation of target cells with 2% TX-100 and media alone, respectively. Antibody independent cellular cytotoxicity (AICC) was measured by incubation of target and effector cells in the absence of antibody. Each assay is performed in triplicate. The mean percentage specific lysis was calculated as: (ADCC−AICC)/(MR−SR)×100.

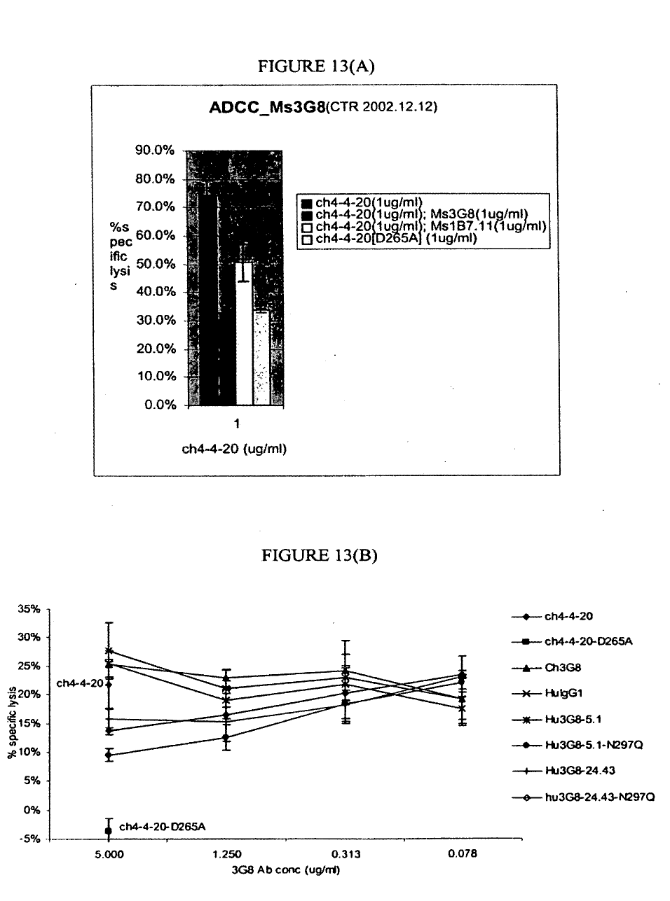

Results: Addition of anti-CD16A variants inhibited ADCC mediated through antibodies directed against the HER2/neu protein (ch4D5) (FIG. 12), or the hapten, fluorescein (ch4-4-20) (FIG. 13). Inhibition of the ch4D5 mediated ADCC was greater than 50% at 300 ng/ml for all 3G8 variants tested while isotype control antibodies had no effect in the assay. In the case of the anti-fluorescein antibody, inhibition was approximately 50% at concentrations above 1 ug/ml for murine 3G8 (FIG. 13A) and humanized 3G8 variants (FIG. 13B), while isotype control antibodies and chimeric 3G8 had little effect.

### Example 15

### Administration of Hu3G8-5.1-N2970 Prevents Immune Thrombocytopenia (ITP) in huFcγRIIa+, huFcγRIIIa+ mice

This example shows that that administration of anti-CD16A antibodies protects against ITP mediated by CD32A. As in FcγRIII−/−, hCD16A mice, administration of the ch6A6 antibody induces ITP in FcγRIII−/−, hCD32A transgenic mice. Five hours after injection of 0.1 μg/g ch6A6 i.p., approximately 80% of the platelets are depleted (not shown). The number of platelets remained low for 24 hours after ch6A6 injection, and then progressively increased to return to normal 48 hours after ch6A6 injection. As expected, the i.v. injection of hu3G8-5.1 (0.5 μg/g) one hour prior to ch6A6 injection did not protect FcγRIII−/−, hCD32A mice against ITP (not shown).

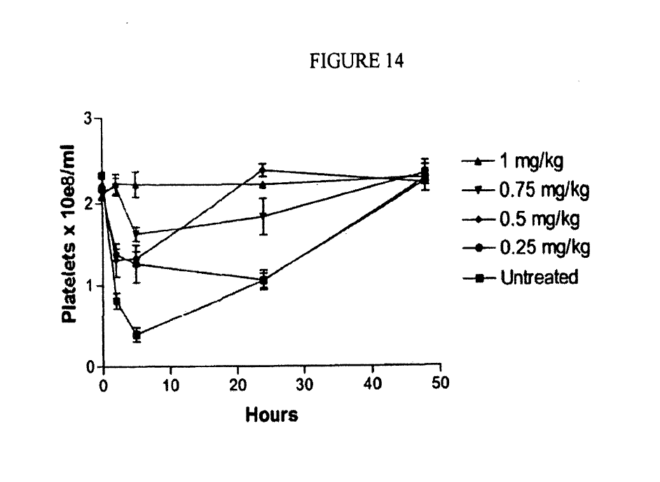

As in single transgenic mice, ch6A6 induces ITP in FcγRIII−/−, hCD16A, hCD32A double transgenic mice. Five hours after injection of 0.1 μg/g ch6A6 i.p., approximately 80% of the platelets were depleted (FIG. 14). The number of platelets remained low for 24 hours after ch6A6 injection, and then progressively increased to return to normal 48 hours after ch6A6 injection.

In contrast to FcγRIII−/−, hCD32A mice, FcγRIII−/−, hCD16A, hCD32A mice were protected against ITP by administration of hu3G8-5.1. Complete protection was observed when 1 μg/g h3G8 5.1 is injected one hour prior to ch6A6 i.p. injection; and partial protection resulted from administration of 0.75 μg/g or 0.5 μg/g of h3G8-5.1 (FIG. 14). Thus, the data indicate that although CD32A can mediate ITP, the injection of 1 μg/g of h3G8-5.1 completely and unexpectedly protects mice against platelet depletion.

### Example 16

### Prevention of Platelet Depletion Using Hu3G8-5.1-N2970 Produced in CHO-S Cell Line

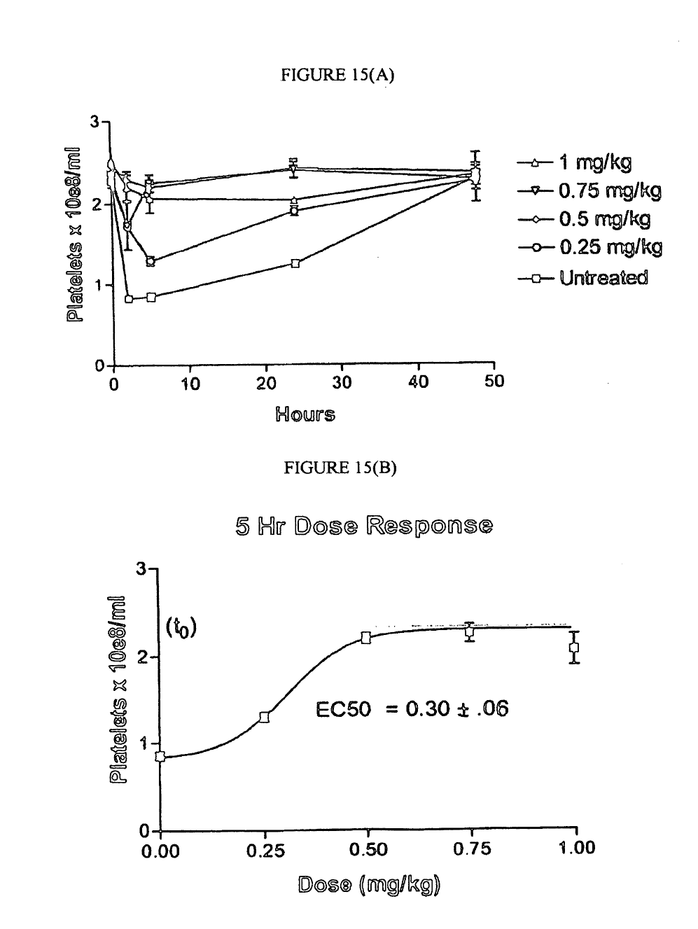

Hu3G8-5.1-N297Q was produced in a CHO-S cell line. The ability of this antibody to protect against ITP in FcγRIII−/−, hCD16A single transgenic mice was determined using the procedure described in Example 13. As is shown in FIG. 15, administration of 0.5 mg/kg or more Hu3G8-5.1-N297Q produced in CHO-S cells one hour prior to ch6A6 i.p. injection completely protects mice against ITP.

### Example 17

### Therapeutic Effect of Aglycosylated Humanized Antibodies

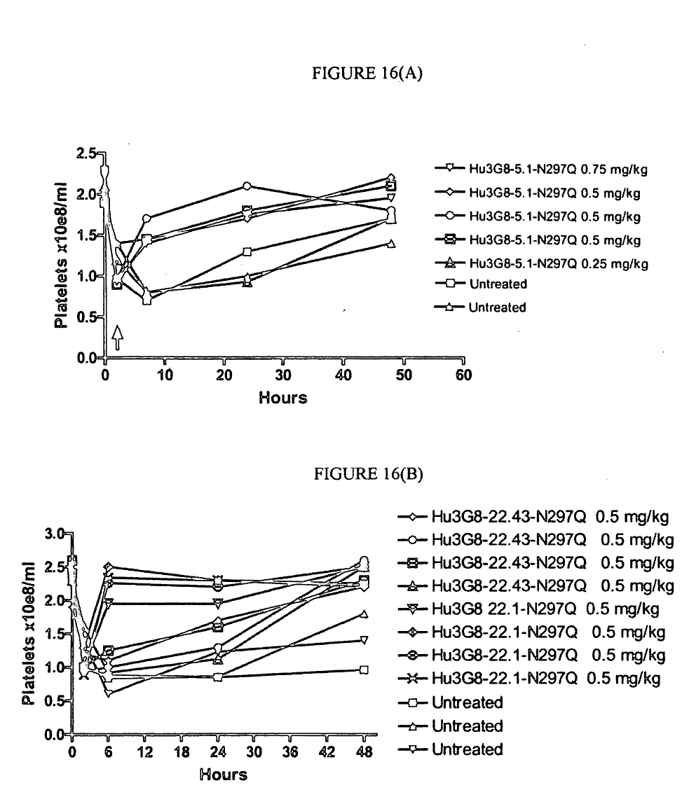

ITP was induced in mice as described above, by i.p. injection of 0.1 μg/g ch6A6 at time 0. Two hours later, the number of platelets in the plasma was determined to confirm the presence of ITP. Three hours after i.p. injection of ch6A6, mice were injected i.v. with hu3G8-5.1-N297Q at different concentration (arrow). The results (FIG. 16A) indicate that the number of platelets rapidly returns to normal after Hu3G8-5.1-N297Q injection whereas the number of platelets remains low in non-treated mice. These results demonstrate that administration of the hu3G8-5.1-N297Q antibody can be used to cure ITP in the mouse model.

In this experiment, ITP was induced by i.p. injection of 0.1 μg/g ch6A6 at time 0. Two hours later, the number of platelets in the plasma was determined to confirm the presence of ITP. Three hours after i.p. injection of ch6A6, mice were injected i.v. with hu3G8-22.1-N297Q or hu3G8-22.43-N297Q at 0.5 μg/g (arrow). The results indicate that the number of platelets rapidly returns to normal after Hu3G8-22.1-N297Q injection whereas the number of platelets remains low in non-treated mice and in mice treated with Hu3G8-22.43-N297Q (FIG. 16B). These data indicate that hu3G8-22.1-N297Q can be used to cure ITP in the mouse model.

### Example 18

### Therapeutic Effect of Hu3G8-22.1-N2970 in AHA in muFcγRIII−/−, huFcγRIIIA Transgenic Mice

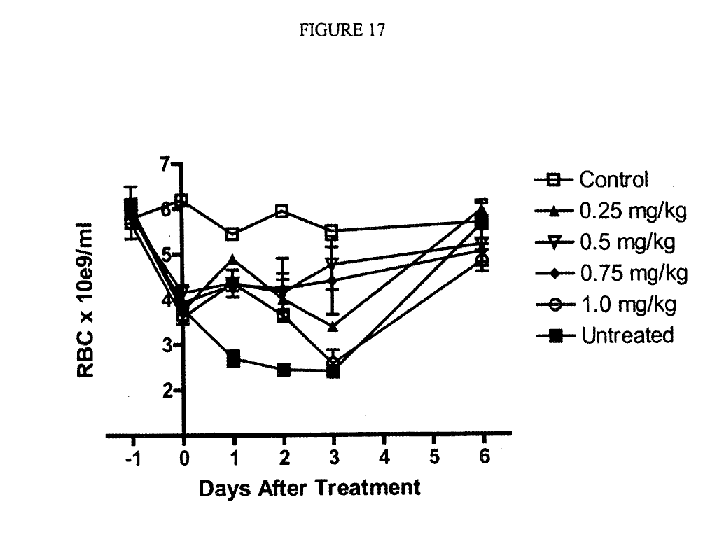

In this experiment, AHA was induced by i.p. injection of 50 ug mouse anti-RBC IgG2a mAb 34-3C at day 0. On day 1, the number of RBC in the blood was determined to confirm the presence of AHA. Two hours later, mice were injected i.v. with Hu3G8-22.1-N297Q at various concentrations (arrow). The results indicate that the number of RBC remained stable after Hu3G8-22.1-N297Q injection whereas the number of RBC continued to drop in non-treated mice (FIG. 17). The optimal concentration of Hu3G8-22.1-N297Q is 0.5 μg/g. The number of RBC returned to normal in all mice at day 7. Control mice were bled every day but not injected in order to determine the effect of repeated bleedings on the number of RBC. These results in the mouse model indicate that Hu3G8-22.1-N297Q can be used to cure AHA. Hu3G8-22.1-N297Q prevents further RBC depletion by autoantibodies and therefore protects mice against anemia.

TABLE 4
TABLE 4A*
V_{H} SEQUENCES

| | FR1 | CDR1 | FR2 | CDR2 | FR3 | CDR3 | FR4 |
|---|---|---|---|---|---|---|---|
| 3G8VH | A | A | A | A | A | A | A |
| Ch3G8VH | A | A | A | A | A | A | B |
| HxC | B | A | B | A | A | A | B |
| CxH | A | A | A | A | B | A | B |
| Hu3G8VH-1 | B | A | B | A | B | A | B |
| Hu3G8VH-2 | C | A | B | A | B | A | B |
| Hu3G8VH-3 | D | A | B | A | B | A | B |
| Hu3G8VH-4 | B | A | B | A | C | B | B |
| Hu3G8VH-5 | B | A | B | A | C | A | B |
| Hu3G8VH-6 | B | B | B | A | B | B | B |
| Hu3G8VH-7 | B | B | B | A | B | A | B |
| Hu3G8VH-8 | B | A | B | A | B | C | B |
| Hu3G8VH-9 | B | A | B | B | B | B | B |
| Hu3G8VH-10 | B | A | B | A | B | B | B |
| Hu3G8VH-11 | B | A | B | B | B | A | B |
| Hu3G8VH-12 | B | A | B | C | B | A | B |
| Hu3G8VH-13 | B | A | B | D | B | A | B |
| Hu3G8VH-14 | B | A | B | E | B | A | B |
| Hu3G8VH-15 | B | A | B | A | D | A | B |
| Hu3G8VH-16 | B | A | B | A | E | A | B |
| Hu3G8VH-17 | B | A | B | A | F | A | B |
| Hu3G8VH-18 | B | A | B | A | G | A | B |
| Hu3G8VH-19 | B | A | B | A | C | C | B |
| Hu3G8VH-20 | B | B | B | C | B | A | B |
| Hu3G8VH-21 | B | A | B | A | D | B | B |
| Hu3G8VH-22 | B | B | B | C | B | C | B |
| Hu3G8VH-23 | B | B | B | C | E | C | B |
| Hu3G8VH-24 | B | B | B | C | F | C | B |
| Hu3G8VH-25 | B | B | B | C | G | C | B |
| Hu3G8VH-26 | B | B | B | C | C | C | B |
| Hu3G8VH-27 | B | B | B | C | E | D | B |
| Hu3G8VH-28 | B | B | B | C | F | D | B |
| Hu3G8VH-29 | B | B | B | C | G | D | B |
| Hu3G8VH-30 | B | B | B | C | C | D | B |
| Hu3G8VH-31 | E | B | B | C | B | A | B |
| Hu3G8VH-32 | E | B | B | H | B | A | B |
| Hu3G8VH-33 | E | B | B | H | B | A | B |
| Hu3G8VH-34 | E | B | B | C | B | C | B |
| Hu3G8VH-35 | E | B | B | C | C | C | B |
| Hu3G8VH-36 | E | B | B | H | C | D | B |
| Hu3G8VH-37 | E | B | B | H | E | C | B |
| Hu3G8VH-38 | E | B | B | F | B | A | B |
| Hu3G8VH-39 | E | B | B | I | B | A | B |
| Hu3G8VH-40 | E | B | B | G | B | A | B |
| Hu3G8VH-41 | E | B | B | J | B | A | B |
| Hu3G8VH-42 | E | B | B | C | H | A | B |
| Hu3G8VH-43 | E | B | B | C | H | C | B |
| Hu3G8VH-44 | E | B | B | C | I | D | B |
| Hu3G8VH-45 | E | B | B | C | J | D | B |

*Letters in Table 4A refer to sequences in Tables 1 B-H.

TABLE 4B
FR1

| A | B | C | D | E | RESIDUE |
|---|---|---|---|---|---|
| Q | Q | Q | Q | Q | 1 |
| V | V | V | V | I | 2 |
| T | T | T | T | T | 3 |
| L | L | L | L | L | 4 |
| K | R | K | R | K | 5 |
| E | E | E | E | E | 6 |
| S | S | S | S | S | 7 |
| G | G | G | G | G | 8 |
| P | P | P | P | P | 9 |
| G | A | A | A | T | 10 |
| I | L | L | L | L | 11 |
| L | V | V | V | V | 12 |
| Q | K | K | K | K | 13 |
| P | P | P | P | P | 14 |
| S | T | T | T | T | 15 |
| Q | Q | Q | Q | Q | 16 |
| T | T | T | T | T | 17 |
| L | L | L | L | L | 18 |
| S | T | T | T | T | 19 |
| L | L | L | L | L | 20 |
| T | T | T | T | T | 21 |
| C | C | C | C | C | 22 |
| S | T | T | T | T | 23 |
| F | F | F | F | F | 24 |
| S | S | S | S | S | 25 |
| G | G | G | G | G | 26 |
| F | F | F | F | F | 27 |
| S | S | S | S | S | 28 |
| L | L | L | L | L | 29 |
| R | S | S | R | S | 30 |
| 30 | 31 | 32 | 33 | 34 | Se ID No |

TABLE 4C
CDR1

| A | B | RESIDUE |
|---|---|---|
| T | T | 31 |
| S | S | 32 |
| G | G | 33 |
| M | V | 34 |
| G | G | 35 |
| V | V | 35A |
| G | G | 35B |
| 35 | 36 | Seq ID No |

TABLE 4D
FR2

| A | B | RESIDUE |
|---|---|---|
| W | W | 36 |
| I | I | 37 |
| R | R | 38 |
| Q | Q | 39 |
| P | P | 40 |
| S | P | 41 |
| G | G | 42 |
| K | K | 43 |
| G | A | 44 |
| L | L | 45 |
| E | E | 46 |
| W | W | 47 |
| L | L | 48 |
| A | A | 49 |
| 37 | 38 | Seq ID No. |

TABLE 4E
CDR2

| A | B | C | D | E | F | G | H | I | J | RESIDUE |
|---|---|---|---|---|---|---|---|---|---|---|
| H | H | H | H | H | L | H | L | H | L | 50 |
| I | I | I | 1 | I | I | I | I | I | I | 51 |
| W | Y | W | Y | W | D | F | W | D | W | 52 |
| W | W | W | W | W | W | W | W | W | W | 53 |
| D | N | D | D | N | D | D | D | D | N | 54 |
| D | D | D | D | D | D | D | D | D | D | 55 |
| D | D | D | D | D | D | D | D | D | D | 56 |
| K | K | K | K | K | K | K | K | K | K | 57 |
| R | R | R | R | R | R | R | R | R | R | 58 |
| Y | Y | Y | Y | Y | Y | Y | Y | Y | Y | 59 |
| N | N | S | N | N | S | S | S | S | S | 60 |
| P | P | P | P | P | P | P | P | P | P | 61 |
| A | A | S | A | A | S | S | S | S | S | 62 |
| L | L | L | L | L | L | L | L | L | L | 63 |
| K | K | K | K | K | K | K | K | K | K | 64 |
| S | S | S | S | S | S | S | S | S | S | 65 |
| 39 | 40 | 41 | 42 | 43 | 44 | 45 | 46 | 47 | 48 | Seq ID No |

TABLE 4F
FR3

| A | B | C | D | E | F | G | H | I | J | RESIDUE |
|---|---|---|---|---|---|---|---|---|---|---|
| R | R | R | R | R | R | R | R | R | R | 66 |
| L | L | L | L | L | L | L | L | L | L | 67 |
| T | T | T | T | T | T | T | T | T | T | 68 |
| I | I | I | I | I | I | I | I | I | I | 69 |
| S | S | S | S | S | S | S | T | T | T | 70 |
| K | K | K | K | K | K | K | K | K | K | 71 |
| D | D | D | D | D | D | D | D | D | D | 72 |
| T | T | T | T | T | T | T | T | T | T | 73 |
| S | S | S | S | S | S | S | S | S | S | 74 |
| S | K | K | K | K | K | K | K | K | K | 75 |
| N | N | N | N | N | N | N | N | N | N | 76. |
| Q | Q | Q | Q | Q | Q | Q | Q | Q | Q | 77 |
| V | V | V | V | V | V | V | V | V | V | 78 |
| F | V | V | V | V | V | V | V | V | V | 79 |
| L | L | L | L | L | L | L | L | L | L | 80 |
| K | T | T | T | T | T | T | T | T | T | 81 |
| I | M | M | M | M | M | M | M | M | M | 82 |
| A | T | T | T | T | T | T | T | T | T | 82A |
| S | N | N | N | N | N | N | N | N | N | 82B |
| V | M | M | M | M | M | M | M | M | M | 82C |
| D | D | D | D | D | D | D | D | D | D | 83 |
| T | P | P | P | P | P | P | P | P | P | 84 |
| A | V | V | V | V | V | V | V | V | V | 85 |
| D | D | D | D | D | D | D | D | D | D | 86 |
| T | T | T | T | T | T | T | T | T | T | 87 |
| A | A | A | A | A | A | A | A | A | A | 88 |
| T | T | T | T | T | T | T | T | T | T | 89 |
| Y | Y | Y | Y | Y | Y | Y | Y | Y | Y | 90 |
| Y | Y | Y | Y | Y | Y | Y | Y | Y | Y | 91 |
| C | C | C | C | C | C | C | C | C | C | 92 |
| A | A | A | A | A | A | A | A | A | A | 93 |
| Q | R | Q | T | K | A | H | R | H | Q | 94 |
| 49 | 50 | 51 | 52 | 53 | 54 | 55 | 56 | 57 | 58 | Seq ID No |

TABLE 4G
CDR3

| A | B | C | D | RESIDUE |
|---|---|---|---|---|
| I | I | I | I | 95 |
| N | N | N | N | 96 |
| P | P | P | P | 97 |
| A | A | A | A | 98 |
| W | W | Y | Y | 99 |
| F | F | F | F | 100 |
| A | D | A | D | 101 |
| Y | Y | Y | Y | 102 |
| 59 | 60 | 61 | 62 | Seq ID No |

TABLE 4H
FR4

| A | B | RESIDUE |
|---|---|---|
| W | W | 103 |
| G | G | 104 |
| Q | Q | 105 |
| G | G | 106 |
| T | T | 107 |
| L | L | 108 |
| V | V | 109 |
| T | T | 110 |
| V | V | 111 |
| S | S | 112 |
| A | S | 113 |
| 63 | 64 | Seq ID No |

TABLE 5
TABLE 5A*
V_{L} SEQUENCES

| | FR1 | CDR1 | FR2 | CDR2 | FR3 | CDR3 | FR4 |
|---|---|---|---|---|---|---|---|
| 3G8VL | A | A | A | A | A | A | A |
| Ch3G8VL | A | A | A | A | A | A | A |
| Hu3G8VL-1 | B | A | A | A | B | A | B |
| Hu3G8VL-2 | B | B | A | A | B | A | B |
| Hu3G8VL-3 | B | C | A | A | B | A | B |
| Hu3G8VL-4 | B | D | A | A | B | A | B |
| Hu3G8VL-5 | B | E | A | A | B | A | B |
| Hu3G8VL-6 | B | F | A | A | B | A | B |
| Hu3G8VL-7 | B | G | A | A | B | A | B |
| Hu3G8VL-8 | B | A | A | B | B | A | B |
| Hu3G8VL-9 | B | A | A | C | B | A | B |
| Hu3G8VL-10 | B | A | A | D | B | A | B |
| Hu3G8VL-11 | B | A | A | E | B | A | B |
| Hu3G8VL-12 | B | A | A | F | B | A | B |
| Hu3G8VL-13 | B | A | A | G | B | A | B |
| Hu3G8VL-14 | B | A | A | A | B | B | B |
| Hu3G8VL-15 | B | A | A | A | B | C | B |
| Hu3G8VL-16 | B | A | A | A | B | D | B |
| Hu3G8VL-17 | B | A | A | A | B | E | B |
| Hu3G8VL-18 | B | B | A | D | B | A | B |
| Hu3G8VL-19 | B | B | A | D | B | D | B |
| Hu3G8VL-20 | B | B | A | D | B | E | B |
| Hu3G8VL-21 | B | C | A | D | B | A | B |
| Hu3G8VL-22 | B | C | A | D | B | D | B |
| Hu3G8VL-23 | B | C | A | D | B | E | B |
| Hu3G8VL-24 | B | D | A | D | B | A | B |
| Hu3G8VL-25 | B | D | A | D | B | D | B |
| Hu3G8VL-26 | B | D | A | D | B | E | B |
| Hu3G8VL-27 | B | E | A | D | B | A | B |
| Hu3G8VL-28 | B | E | A | D | B | D | B |
| Hu3G8VL-29 | B | E | A | D | B | E | B |
| Hu3G8VL-30 | B | A | A | D | B | D | B |
| Hu3G8VL-31 | B | A | A | D | B | E | B |
| Hu3G8VL-32 | B | A | A | H | B | A | B |
| Hu3G8VL-33 | B | A | A | I | B | A | B |
| Hu3G8VL-34 | B | A | A | J | B | A | B |
| Hu3G8VL-35 | B | B | A | H | B | D | B |
| Hu3G8VL-36 | B | C | A | H | B | D | B |
| Hu3G8VL-37 | B | E | A | H | B | D | B |
| Hu3G8VL-38 | B | B | A | I | B | D | B |
| Hu3G8VL-39 | B | C | A | I | B | D | B |
| Hu3G8VL-40 | B | E | A | I | B | D | B |
| Hu3G8VL-41 | B | B | A | J | B | D | B |
| Hu3G8VL-42 | B | C | A | J | B | D | B |
| Hu3G8VL-43 | B | E | A | J | B | D | B |
| Hu3G8VL-44 | B | A | A | K | B | A | B |

*Letters in Table 5A refer to sequences in Tables 3B-H.

TABLE 5B
FR1

| A | B | RESIDUE |
|---|---|---|
| D | D | 1 |
| T | I | 2 |
| V | V | 3 |
| L | M | 4 |
| T | T | 5 |
| Q | Q | 6 |
| S | S | 7 |
| P | P | 8 |
| A | D | 9 |
| S | S | 10 |
| L | L | 11 |
| A | A | 12 |
| V | V | 13 |
| S | S | 14 |
| L | L | 15 |
| G | G | 16 |
| Q | E | 17 |
| R | R | 18 |
| A | A | 19 |
| T | T | 20 |
| I | I | 21 |
| S | N | 22 |
| C | C | 23 |
| 65 | 66 | Seq ID No |

TABLE 5C
CDR1

| A | B | C | D | E | F | G | RESIDUE |
|---|---|---|---|---|---|---|---|
| K | R | K | K | K | K | K | 24 |
| A | A | S | A | A | A | A | 25 |
| S | S | S | S | S | S | S | 26 |
| Q | Q | Q | Q | Q | Q | Q | 27 |
| S | S | S | S | S | S | S | 27A |
| V | V | V | V | V | V | V | 27B |
| D | D | D | D | D | D | D | 27C |
| F | F | F | F | F | F | F | 27D |
| D | D | D | D | D | D | D | 28 |
| G | G | G | G | G | G | G | 29 |
| D | D | D | D | D | D | D | 30 |
| S | S | S | S | S | S | S | 31 |
| F | F | F | Y | F | F | Y | 32 |
| M | M | M | M | L | M | L | 33 |
| N | N | N | N | N | A | A | 34 |
| 67 | 68 | 69 | 70 | 71 | 72 | 73 | Seq ID No |

TABLE 5D
FR2

| A | RESIDUE |
|---|---|
| W | 35 |
| Y | 36 |
| Q | 37 |
| Q | 38 |
| K | 39 |
| P | 40 |
| G | 41 |
| Q | 42 |
| P | 43 |
| P | 44 |
| K | 45 |
| L | 46 |
| L | 47 |
| I | 48 |
| Y | 49 |
| 74 | Seq ID No |

TABLE 5E
CDR2

| A | B | C | D | E | F | G | H | I | J | K | RESIDUE |
|---|---|---|---|---|---|---|---|---|---|---|---|
| T | D | W | T | D | D | S | S | S | T | T | 50 |
| T | A | A | T | A | A | A | T | T | T | T | 51 |
| S | S | S | S | S | S | S | S | S | S | S | 52 |
| N | N | N | N | N | N | N | N | N | N | S | 53 |
| L | L | L | L | L | L | L | L | L | L | L | 54 |
| E | E | E | E | E | A | Q | E | Q | Q | Q | 55 |
| S | S | S | T | T | T | S | S | S | S | S | 56 |
| 75 | 76 | 77 | 78 | 79 | 80 | 81 | 82 | 83 | 84 | 85 | Seq ID No |

TABLE 5F
FR3

| A | B | RESIDUE |
|---|---|---|
| G | G | 57 |
| I | V | 58 |
| P | P | 59 |
| A | D | 60 |
| R | R | 61 |
| F | F | 62 |
| S | S | 63 |
| A | G | 64 |
| S | S | 65 |
| G | G | 66 |
| S | S | 67 |
| G | G | 68 |
| T | T | 69 |
| D | D | 70 |
| F | F | 71 |
| T | T | 72 |
| L | L | 73 |
| N | T | 74 |
| I | I | 75 |
| H | S | 76 |
| P | S | 77 |
| V | L | 78 |
| E | Q | 79 |
| E | A | 80 |
| E | E | 81 |
| D | D | 82 |
| T | V | 83 |
| A | A | 84 |
| T | V | 85 |
| Y | Y | 86 |
| Y | Y | 87 |
| C | C | 88 |
| 86 | 87 | Seq ID No |

TABLE 5G
CDR3

| A | B | C | D | E | RESIDUE |
|---|---|---|---|---|---|
| Q | Q | Q | Q | Q | 89 |
| Q | Q | Q | Q | Q | 90 |
| S | S | S | S | S | 91 |
| N | Y | Y | N | N | 92 |
| E | S | E | S | E | 93 |
| D | T | D | D | T | 94 |
| P | P | P | P | P | 95 |
| Y | Y | Y | Y | Y | 96 |
| T | T | T | T | T | 97 |
| 88 | 89 | 90 | 91 | 92 | Seq ID No |

TABLE 5H
FR4

| A | B | RESIDUE |
|---|---|---|
| F | F | 98 |
| G | G | 99 |
| G | Q | 100 |
| G | G | 101 |
| T | T | 102 |
| K | K | 103 |
| L | L | 104 |
| E | E | 105 |
| I | I | 106 |
| K | K | 107 |
| 93 | 94 | Seq ID No |

TABLE 6
Hu3G8VL-1

(SEQ ID NO:95)

CGAGCTAGCTGAGATCACAGTTCTCTCTACAGTTACTGAGCACACAGGAC
CTCACCATGGGATGGAGCTGTATCATCCTCTTCTTGGTAGCAACAGCTAC
AGGTAAGGGGCTCACAGTAGCAGGCTTGAGGTCTGGACATATATATGGGT
GACAATGACATCCACTTTGCCTTTCTCTCCACAGGTGTCCACTCCGACAT
CGTGATGACCCAATCTCCAGACTCTTTGGCTGTGTCTCTAGGGGAGAGGG
CCACCATCAACTGCAAGGCCAGCCAAACTGTTGATTTTGATGGTGATAGT
TTTATGAACTGGTACCAACAGAAACCAGGACAGCCACCCAAACTCCTCAT
CTATACTACATCCAATCTAGAATCTGGGGTCCCAGACAGGTTTAGTGGCA
GTGGGTCTGGGACAGACTTCACCCTCACCATCAGCAGCCTGCAGGCTGAG
GATGTGGCAGTTTATTACTGTCAGCAAAGTAATGAGGATCCGTACACGTT
CGGACAGGGGACCAAGCTTGAgATcAAA
Hu3G8VL-1

(SEQ ID NO:96)

DIVMTQSPDSLAVSLGERATINCKASQSVDFDGDSFMNWYQQKPGQPPKL
LIYTTSNLESGVPDRFSGSGSGTDFTLTISSLQAEDVAVYYCQQSNEDPY
TFGQGTKLEIK
Hu3G8VL-1K

(SEQ ID NO:97)

CGAGCTAGCTGAGATCACAGTTCTCTCTACAGTTACTGAGCACACAGGAC
CTCACCATGGGATGGAGCTGTATCATCCTCTTCTTGGTAGCAACAGCTAC
AGGTAAGGGGCTCACAGTAGCAGGCTTGAGGTCTGGACATATATATGGGT
GACAATGACATCCACTTTGCCTTTCTCTCCACAGGTGTCCACTCCGACAT
CGTGATGACCCAATCTCCAGACTCTTTGGCTGTGTCTCTAGGGGAGAGGG
CCACCATCAACTGCAAGGCCAGCCAAAGTGTTGATTTTGATGGTGATAGT
TTTATGAACTGGTACCAACAGAAACCAGGACAGCCACCCAAACTCCTCAT
CTATACTACATCCAATCTAGAATCTGGGGTCCCAGACAGGTTTAGTGGCA
GTGGGTCTGGGACAGACTTCACCCTCACCATCAGCAGCCTGCAGGCTGAG
GATGTGGCAGTTTATTACTGTCAGCAAAGTAATGAGGATCCGTACACGTT
CGGACAGGGGACCAAGCTTGAgATcAAACGaACTGTGGCTGCACCATCGG
TCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAG
AGCAGGACAGCAAGGACAGCACCTACAGCCTCAGCAGCACCCTGACGCTG
AGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTT
AGTTCTAGAGTCGACTCTAGAGGATCCCCGGGTACCGAGCTCGAATTC
Hu3G8VL-1K

(SEQ ID NO:98)

DIVMTQSPDSLAVSLGERATINCKASQSVDFDGDSFMNWYQQKPGQPPKL
LIYTTSNLESGVPDRFSGSGSGTDFTLTISSLQAEDVAVYYCQQSNEDPY
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
Hu3G8VL-43

(SEQ ID NO:99)

CGAGCTAGCTGAGATCACAGTTCTCTCTACAGTTACTGAGCACACAGGAC
CTCACCATGGGATGGAGCTGTATCATCCTCTTCTTGGTAGCAACAGCTAC
AGGTAAGGGGCTCACAGTAGCAGGCTTGAGGTCTGGACATATATATGGGT
GACAATGACATCCACTTTGCCTTTCTCTCCACAGGTGTCCACTCCGACAT
CGTGATGACCCAATCTCCAGACTCTTTGGCTGTGTCTCTAGGGGAGAGGG
CCACCATCAACTGCAAGtCCAGCCAAAGTGTTGATTTTGATGGTGATAGT
TTTATGAACTGGTACCAACAGAAACCAGGACAGCCACCCAAACTCCTCAT
CTATACTACATCCAgTCTAGAATCTGGGGTCCCAGACAGGTTTAGTGGCA
GTGGGTCTGGGACAGACTTCACCCTCACCATCAGCAGCCTGCAGGCTGAG
GATGTGGCAGTTTATTACTGTCAGCAAAGTAATtcGGATCCGTACACGTT
CGGACAGGGGACCAAGCTTGAgATcAAA
Hu3G8VL-43

(SEQ ID NO:100)

DIVMTQSPDSLAVSLGERATINCKSSQSVDFDGDSFMNWYQQKPGQPPKL
LIYTTSSLESGVPDRFSGSGSGTDFTLTISSLQAEDVAVYYCQQSNSDPY
TFGQGTKLEIK
Hu3G8VL-43 + Kappa

(SEQ ID NO:101)

CGAGCTAGCTGAGATCACAGTTCTCTCTACAGTTACTGAGCACACAGGAC
CTCACCATGGGATGGAGCTGTATCATCCTCTTCTTGGTAGCAACAGCTAC
AGGTAAGGGGCTCACAGTAGCAGGCTTGAGGTCTGGACATATATATGGGT
GACAATGACATCCACTTTGCCTTTCTCTCCACAGGTGTCCACTCCGACAT
CGTGATGACCCAATCTCCAGACTCTTTGGCTGTGTCTCTAGGGGAGAGGG
CCACCATCAACTGCAAGtCCAGCCAAAGTGTTGATTTTGATGGTGATAGT
TTTATGAACTGGTACCAACAGAAACCAGGACAGCCACCCAAACTCCTCAT
CTATACTACATCCAgTCTAGAATCTGGGGTCCCAGACAGGTTTAGTGGCA
GTGGGTCTGGGACAGACTTCACCCTCACCATCAGCAGCCTGCAGGCTGAG
GATGTGGCAGTTTATTACTGTCAGCAAAGTAATtcGGATCCGTACACGTT
CGGACAGGGGACCAAGCTTGAgATcAAACGaACTGTGGCTGCACCATCGG
TCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAG
AGCAGGACAGCAAGGACAGCACCTACAGCCTCAGCAGCACCCTGACGCTG
AGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTT
AGTTCTAGAGTCGACTCTAGAGGATCCCCGGGTACCGAGCTCGAATTC
Hu3G8VL-43K

(SEQ ID NO:102)

DIVMTQSPDSLAVSLGERATINCKSSQSVDFDGDSFMNWYQQKPGQPPKL
LIYTTSSLESGVPDRFSGSGSGTDFTLTISSLQAEDVAVYYCQQSNSDPY
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
Hu3G8VH-1

(SEQ ID NO:103)

GCTAGCgtttaaacttaagcttGTTGACTAGTGAGATCACAGTTCTCTCT
ACAGTTACTGAGCACACAGGACCTCACCATGGGATGGAGCTGTATCATCC
TCTTCTTGGTAGCAACAGCTACAGGTAAGGGGCTCACAGTAGCAGGCTTG
AGGTCTGGACATATATATGGGTGACAATGACATCCACTTTGCCTTTCTCT
CCACAGGTGTCCACTCCCAGGTTACCCTGAGAGAGTCTGGCCCTGCGCTG
GTGAAGCCCACACAGACCCTCACACTGACTTGTACCTTCTCTGGGTTTTC
ACTGAGCACTTCTGGTATGGGTGTAGGCTGGATTCGTCAGCCTCCCGGGA
AGGCTCTAGAGTGGCTGGCACACATTTGGTGGGATGATGACAAGCGCTAT
AATCCAGCCCTGAAGAGCCGACTGACAATCTCCAAGGATACCTCCAAAAA
CCAGGTAGTCCTCACAATGACCAACATGGACCCTGTGGATACTGCCACAT
ACTACTGTGCTCGGATAAACCCCGCCTGGTTTGCTTACTGGGGCCAAGGG
ACTCTGGTCACTGTGAGCTCA
Hu3G8VH-1

(SEQ ID NO:104)

QVTLRESGPALVKPTQTLTLTCTFSGFSLSTSGMGVGWIRQPPGKALEWL
AHIWWDDDKRYNPALKSRLTISKDTSKNQVVLTMTNMDPVDTATYYCART
NPAWFAYWGQGTLVTVSS
Hu3G8VH-1G1

(SEQ ID NO:105)

GCTAGCgtttaaacttaagcttGTTGACTAGTGAGATCACAGTTCTCTCT
ACAGTTACTGAGCACACAGGACCTCACCATGGGATGGAGCTGTATCATCC
TCTTCTTGGTAGCAACAGCTACAGGTAAGGGGCTCACAGTAGCAGGCTTG
AGGTCTGGACATATATATGGGTGACAATGACATCCACTTTGCCTTTCTCT
CCACAGGTGTCCACTCCCAGGTTACCCTGAGAGAGTCTGGCCCTGCGCTG
GTGAAGCCCACACAGACCCTCACACTGACTTGTACCTTCTCTGGGTTTTC
ACTGAGCACTTCTGGTATGGGTGTAGGCTGGATTCGTCAGCCTCCCGGGA
AGGCTCTAGAGTGGCTGGCACACATTTGGTGGGATGATGACAAGCGCTAT
AATCCAGCCCTGAAGAGCCGACTGACAATCTCCAAGGATACCTCCAAAAA
CCAGGTAGTCCTCACAATGACCAACATGGACCCTGTGGATACTGCCACAT
ACTACTGTGCTCGGATAAACCCCGCCTGGTTTGCTTACTGGGGCCAAGGG
ACTCTGGTCACTGTGAGCTCAgcctccaccaagggcccatcggtcttccc
cctggcaccctcctccaagagcacctctgggggcacagcggccctgggct
gcctggtcaaggactacttccccgaaccggtgacggtgtcgtggaactca
ggcgccctgaccagcggcgtgcacaccttcccggctgtcctacagtcctc
aggactctactccctcagcagcgtggtgaccgtgccctccagcagcttgg
gcacccagacctacatctgcaacgtgaatcacaagcccagcaacaccaag
gtggacaagagagttgagcccaaatcttgtgacaaaactcacacatgccc
accgtgcccagcacctgaactcctggggggaccgtcagtcttcctcttcc
ccccaaaacccaaggacaccctcatgatctcccggacccctgaggtcaca
tgcgtggtggtggacgtgagccacgaagaccctgaggtcaagttcaactg
gtacgtggacggcgtggaggtgcataatgccaagacaaagccgcgggagg
agcagtacaacagcacgtaccgtgtggtcagcgtcctcaccgtcctgcac
caggactggctgaatggcaaggagtacaagtgcaaggtctccaacaaagc
cctcccagcccccatcgagaaaaccatctccaaagccaaagggcagcccc
gagaaccacaggtgtacaccctgcccccatcccgggatgagctgaccaag
aaccaggtcagcctgacctgcctggtcaaaggcttctatcccagcgacat
cgccgtggagtgggagagcaatgggcagccggagaacaactacaagacca
cgcctcccgtgctggactccgacggctccttcttcctctacagcaagctc
accgtggacaagagcaggtggcagcaggggaacgtcttctcatgctccgt
gatgcatgaggctctgcacaaccactacacgcagaagagcctctccctgt
ctccgggtaaatgagtgcggccgcGAATTC
Hu3G8VH-1G1

(SEQ ID NO:107)

QVTLRESGPALVKPTQTLTLTCTFSGFSLSTSGMGVGWIRQPPGKALEWL
AHIWWDDDKRYNPALKSRLTISKDTSKNQVVLTMTNMDPVDTATYYCARI
NPAWFAYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
Hu3G8VH-5

(SEQ ID NO:108)

GCTAGCgtttaaacttaagcttGTTGACTAGTGAGATCACAGTTCTCTCT
ACAGTTACTGAGCACACAGGACCTCACCATGGGATGGAGCTGTATCATCC
TCTTCTTGGTAGCAACAGCTACAGGTAAGGGGCTCACAGTAGCAGGCTTG
AGGTCTGGACATATATATGGGTGACAATGACATCCACTTTGCCTTTCTCT
CCACAGGTGTCCACTCCCAGGTTACCCTGAGAGAGTCTGGCCCTGCGCTG
GTGAAGCCCACACAGACCCTCACACTGACTTGTACCTTCTCTGGGTTTTC
ACTGAGCACTTCTGGTATGGGTGTAGGCTGGATTCGTCAGCCTCCCGGGA
AGGCTCTAGAGTGGCTGGCACACATTTGGTGGGATGATGACAAGCGCTAT
AATCCAGCCCTGAAGAGCCGACTGACAATCTCCAAGGATACCTCCAAAAA
CCAGGTAGTCCTCACAATGACCAACATGGACCCTGTGGATACTGCCACAT
ACTACTGTGCTCaaATAAACCCCGCCTGGTTTGCTTACTGGGGCCAAGGG
ACTCTGGTCACTGTGAGCTCA
Hu3G8VH-5

(SEQ ID NO:109)

QVTLRESGPALVKPTQTLTLTCTFSGFSLSTSGMGVGWIRQPPGKALEWL
AHIWWDDDKRYNPALKSRLTISKDTSKNQVVLTMTNMDPVDTATYYCAQI
NPAWFAYWGQGTLVTVSS
Hu3G8VH-5G1Ag

(SEQ ID NO:110)

GCTAGCgtttaaacttaagcttGTTGACTAGTGAGATCACAGTTCTCTCT
ACAGTTACTGAGCACACAGGACCTCACCATGGGATGGAGCTGTATCATCC
TCTTCTTGGTAGCAACAGCTACAGGTAAGGGGCTCACAGTAGCAGGCTTG
AGGTCTGGACATATATATGGGTGACAATGACATCCACTTTGCCTTTCTCT
CCACAGGTGTCCACTCCCAGGTTACCCTGAGAGAGTCTGGCCCTGCGCTG
GTGAAGCCCACACAGACCCTCACACTGACTTGTACCTTCTCTGGGTTTTC
ACTGAGCACTTCTGGTATGGGTGTAGGCTGGATTCGTCAGCCTCCCGGGA
AGGCTCTAGAGTGGCTGGCACACATTTGGTGGGATGATGACAAGCGCTAT
AATCCAGCCCTGAAGAGCCGACTGACAATCTCCAAGGATACCTCCAAAAA
CCAGGTAGTCCTCACAATGACCAACATGGACCCTGTGGATACTGCCACAT
ACTACTGTGCTCaaATAAACCCCGCCTGGTTTGCTTACTGGGGCCAAGGG
ACTCTGGTCACTGTGAGCTCAgcctccaccaagggcccatcggtcttccc
cctggcaccctcctccaagagcacctctgggggcacagcggccctgggct
gcctggtcaaggactacttccccgaaccggtgacggtgtcgtggaactca
ggcgccctgaccagcggcgtgcacaccttcccggctgtcctacagtcctc
aggactctactccctcagcagcgtggtgaccgtgccctccagcagcttgg
gcacccagacctacatctgcaacgtgaatcacaagcccagcaacaccaag
gtggacaagagagttgagcccaaatcttgtgacaaaactcacacatgccc
accgtgcccagcacctgaactcctggggggaccgtcagtcttcctcttcc
ccccaaaacccaaggacaccctcatgatctcccggacccctgaggtcaca
tgcgtggtggtggacgtgagccacgaagaccctgaggtcaagttcaactg
gtacgtggacggcgtggaggtgcataatgccaagacaaagccgcgggagg
agcagtacCaGagcacgtaccgtgtggtcagcgtcctcaccgtcctgcac
caggactggctgaatggcaaggagtacaagtgcaaggtctccaacaaagc
cctcccagcccccatcgagaaaaccatctccaaagccaaagggcagcccc
gagaaccacaggtgtacaccctgcccccatcccgggatgagctgaccaag
aaccaggtcagcctgacctgcctggtcaaaggcttctatcccagcgacat
cgccgtggagtgggagagcaatgggcagccggagaacaactacaagacca
cgcctcccgtgctggactccgacggctccttcttcctctacagcaagctc
accgtggacaagagcaggtggcagcaggggaacgtcttctcatgctccgt
gatgcatgaggctctgcacaaccactacacgcagaagagcctctccctgt
ctccgggtaaatgagtgcggccgcGAATTC
Hu3G8VH-5G1Ag

(SEQ ID NO:111)

QVTLRESGPALVKPTQTLTLTCTFSGFSLSTSGMGVGWIRQPPGKALEWL
AHIWWDDDKRYNPALKSRLTISKDTSKNQVVLTMTNMDPVDTATYYCAQI
NPAWFAYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYQST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
Hu3G8VH-22

(SEQ ID NO:112)

GCTAGCgtttaaacttaagcttGTTGACTAGTGAGATCACAGTTCTCTCT
ACAGTTACTGAGCACACAGGACCTCACCATGGGATGGAGCTGTATCATCC
TCTTCTTGGTAGCAACAGCTACAGGTAAGGGGCTCACAGTAGCAGGCTTG
AGGTCTGGACATATATATGGGTGACAATGACATCCACTTTGCCTTTCTCT
CCACAGGTGTCCACTCCCAGGTTACCCTGAGAGAGTCTGGCCCTGCGCTG
GTGAAGCCCACACAGACCCTCACACTGACTTGTACCTTCTCTGGGTTTTC
ACTGAGCACTTCTGGTgTGGGTGTAGGCTGGATTCGTCAGCCTCCCGGGA
AGGCTCTAGAGTGGCTGGCACACATTTGGTGGGATGATGACAAGCGCTAT
tcTCCAtCCCTGAAGAGCCGACTGACAATCTCCAAGGATACCTCCAAAAA
CCAGGTAGTCCTCACAATGACCAACATGGACCCTGTGGATACTGCCACAT
ACTACTGTGCTCGGATAAACCCCGCCTacTTTGCTTACTGGGGCCAAGGG
ACTCTGGTCACTGTGAGCTCA
Hu3G8VH-22

(SEQ ID NO:113)

QVTLRESGPALVKPTQTLTLTCTFSGFSLSTSGVGVGWIRQPPGKALEWL
AHIWWDDDKRYSPSLKSRLTISKDTSKNQVVLTMTNMDPVDTATYYCARI
NPAYFAYWGQGTLVTVS
Hu3G8VH-22G1Ag

(SEQ ID NO:114)

GCTAGCgtttaaacttaagcttGTTGACTAGTGAGATCACAGTTCTCTCT
ACAGTTACTGAGCACACAGGACCTCACCATGGGATGGAGCTGTATCATCC
TCTTCTTGGTAGCAACAGCTACAGGTAAGGGGCTCACAGTAGCAGGCTTG
AGGTCTGGACATATATATGGGTGACAATGACATCCACTTTGCCTTTCTCT
CCACAGGTGTCCACTCCCAGGTTACCCTGAGAGAGTCTGGCCCTGCGCTG
GTGAAGCCCACACAGACCCTCACACTGACTTGTACCTTCTCTGGGTTTTC
ACTGAGCACTTCTGGTgTGGGTGTAGGCTGGATTCGTCAGCCTCCCGGGA
AGGCTCTAGAGTGGCTGGCACACATTTGGTGGGATGATGACAAGCGCTAT
tcTCCAtCCCTGAAGAGCCGACTGACAATCTCCAAGGATACCTCCAAAAA
CCAGGTAGTCCTCACAATGACCAACATGGACCCTGTGGATACTGCCACAT
ACTACTGTGCTCGGATAAACCCCGCCTacTTTGCTTACTGGGGCCAAGGG
ACTCTGGTCACTGTGAGCTCAgcctccaccaagggcccatcggtcttccc
cctggcaccctcctccaagagcacctctgggggcacagcggccctgggct
gcctggtcaaggactacttccccgaaccggtgacggtgtcgtggaactca
ggcgccctgaccagcggcgtgcacaccttcccggctgtcctacagtcctc
aggactctactccctcagcagcgtggtgaccgtgccctccagcagcttgg
gcacccagacctacatctgcaacgtgaatcacaagcccagcaacaccaag
gtggacaagagagttgagcccaaatcttgtgacaaaactcacacatgccc
accgtgcccagcacctgaactcctggggggaccgtcagtcttcctcttcc
ccccaaaacccaaggacaccctcatgatctcccggacccctgaggtcaca
tgcgtggtggtggacgtgagccacgaagaccctgaggtcaagttcaactg
gtacgtggacggcgtggaggtgcataatgccaagacaaagccgcgggagg
agcagtacCaGagcacgtaccgtgtggtcagcgtcctcaccgtcctgcac
caggactggctgaatggcaaggagtacaagtgcaaggtctccaacaaagc
cctcccagcccccatcgagaaaaccatctccaaagccaaagggcagcccc
gagaaccacaggtgtacaccctgcccccatcccgggatgagctgaccaag
aaccaggtcagcctgacctgcctggtcaaaggcttctatcccagcgacat
cgccgtggagtgggagagcaatgggcagccggagaacaactacaagacca
cgcctcccgtgctggactccgacggctccttcttcctctacagcaagctc
accgtggacaagagcaggtggcagcaggggaacgtcttctcatgctccgt
gatgcatgaggctctgcacaaccactacacgcagaagagcctctccctgt
ctccgggtaaatgagtgcggccgcGAATTC
Hu3G8VH-22G1Ag

(SEQ ID NO:115)

QVTLRESGPALVKPTQTLTLTCTFSGFSLSTSGVGVGWIRQPPGKALEWL
AHIWWDDDKRYSPSLKSRLTISKDTSKNQVVLTMTNMDPVDTATYYCARI
NPAYFAYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYQST
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK
Hu3G8VL-22

(SEQ ID NO:118)

DIVMTQSPDSLAVSLGERATINCKSSQSVDFDGDSFMNWYQQKPGQPPKL
LIYTTSNLETGVPDRFSGSGSGTDFTLTISSLQAEDVAVYYCQQSNSDPY
TFGQGTKLEIK
Hu3G8VL-22K

(SEQ ID NO:119)

DIVMTQSPDSLAVSLGERATINCKSSQSVDFDGDSFMNWYQQKPGQPPKL
LIYTTSNLETGVPDRFSGSGSGTDFTLTISSLQAEDVAVYYCQQSNSDPY
TFGQGTKLEIKRTVAAPSVFTFPPSDEQLKSGTASVVCLLNNEYPREAKV
QWKVDNALQSGNSQESVTEQDSKDSTYSLSSTLTLSKADYEKHKVYACEV
THQGLSSPVTKSFNRGEC
Hu3G8VL-22

(SEQ ID NO:106)

CGAGCTAGCTGAGATCACAGTTCTCTCTACAGTTACTGAGCACACAGGAC
CTCACCATGGGATGGAGCTGTATCATCCTCTTCTTGGTAGCAACAGCTAC
AGGTAAGGGGCTCACAGTAGCAGGCTTGAGGTCTGGACATATATATGGGT
GACAATGACATCCACTTTGCCTTTCTCTCCACAGGTGTCCACTCCGACAT
CGTGATGACCCAATCTCCAGACTCTTTGGCTGTGTCTCTAGGGGAGAGGG
CCACCATCAACTGCAAGTCCAGCCAAAGTGTTGATTTTGATGGTGATAGT
TTTATGAACTGGTACCAACAGAAACCAGGACAGCCACCCAAACTCCTCAT
CTATACTACATCCAATCTAGAAACTGGGGTCCCAGACAGGTTTAGTGGCA
GTGGGTCTGGGACAGACTTCACCCTCACCATCAGCAGCCTGCAGGCTGAG
GATGTGGCAGTTTATTACTGTCAGCAAAGTAATTCGGATCCGTACACGTT
CGGACAGGGGACCAAGCTTGAgATcAAA
Hu3G8VL-22K

(SEQ ID NO:24)

CGAGCTAGCTGAGATCACAGTTCTCTCTACAGTTACTGAGCACACAGGAC
CTCACCATGGGATGGAGCTGTATCATCCTCTTCTTGGTAGCAACAGCTAC
AGGTAAGGGGCTCACAGTAGCAGGCTTGAGGTCTGGACATATATATGGGT
GACAATGACATCCACTTTGCCTTTCTCTCCACAGGTGTCCACTCCGACAT
CGTGATGACCCAATCTCCAGACTCTTTGGCTGTGTCTCTAGGGGAGAGGG
CCACCATCAACTGCAAGTCCAGCCAAAGTGTTGATTTTGATGGTGATAGT
TTTATGAACTGGTACCAACAGAAACCAGGACAGCCACCCAAACTCCTCAT
CTATACTACATCCAATCTAGAAACTGGGGTCCCAGACAGGTTTAGTGGCA
GTGGGTCTGGGACAGACTTCACCCTCACCATCAGCAGCCTGCAGGCTGAG
GATGTGGCAGTTTATTACTGTCAGCAAAGTAATTCGGATCCGTACACGTT
CGGACAGGGGACCAAGCTTGAgATcAAACGaACTGTGGCTGCACCATCGG
TCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAG
AGCAGGACAGCAAGGACAGCACCTACAGCCTCAGCAGCACCCTGACGCTG
AGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTT
AGTTCTAGAGTCGACTCTAGAGGATCCCCGGGTACCGAGCTCGAATTC

### Example 18

### A Phase I, Open-Label, Two-Center, Single-Dose, Dose-Escalating, Safety, Tolerability, Immunogenicity Study of GMA-161 in Patients with Idiopathic Thrombocytopenic Purpura (ITP)

GMA-161, a humanized antibody to the Fcγ receptor III (FcγRIII, CD16A), is being developed to block the phagocytosis by splenic and hepatic macrophages of antibody-coated platelets. Several clinical studies have been conducted using a murine anti-CD16A mAb 3G8 in patients with refractory ITP. Although a rise in platelet counts was observed in the majority of patients, cytokine release syndrome, transient neutropenia, and human anti-murine antibody (HAMA) responses precluded further clinical development. In addition to being ‘humanized’, GMA-161 has been modified from 3G8 to decrease the ability of its Fc domain to fix complement or bind to receptors on lymphocytes, neutrophils or NK cells.

Patients: The patients will have been diagnosed with ITP for at least 6 months. Preferably the patient will have a platelet count of <50,000/mm³ on 2 determinations at least 6 weeks apart, including 1 determination within 7 days prior to initiating study treatment.

Administration of GAM-161: The patient will receive a single IV infusion of GMA-161 on Day 0 and will be monitored for seven days with collection of blood samples and for safety observation. The patient will receive treatment with GMA-161 dosed at 0.1 mg/kg body weight. GMA-161 is a liquid solution with a protein concentration of 5 mg/mL (25 mg/vial). GMA-161 is in a buffer composed of 5 mM sodium phosphate and 1.7 mM potassium phosphate at pH 7.2, containing 154 mM sodium chloride.

Immunological and Platelet count Assessments: Samples for immunology assessments will be collected various time points specified below: Platelet counts will also be monitored. Blood samples will be collected 2 hrs, 5 hrs, 24 hrs and 48 hrs after administration of GMA-161 and plasma platelet counts will be determined using a particle count and size analyzer Z2™ COULTER COUNTER® equipped with a 70 μm aperture. Data is analyzed by plotting the relative platelet level (the actual platelet count divided by the time 0 platelet count) versus time for each concentration.

Day 0 (pre-infusion), Day 7, Day 21 and Day 28: Anti-platelet assay, antinuclear antibodies (ANA), anti-double stranded DNA antibodies, anticardiolipin antibodies (ACA), lymphocyte subsets (including natural killer cells), and serum immunoglobulin (IgG, IgA, and IgM) concentrations, and serum cytokine assays (e.g., interleukin-6 [IL-6], IL-8, and tumor necrosis factor alpha [TNFα]). If a patient exhibits signs or symptoms of cytokine release syndrome, blood will be collected at 30 minutes post infusion for cytokine assays.

Day 0 (pre-infusion) and Day 7: Serum CD16A and C3 and C4 complement. Pre-infusion anti-GMA-161 antibody serum will be collected at Day 0. Post-infusion anti-GMA-161 antibody assay will start when the blood GMA-161 level no longer interferes with the assay, as determined by the Genzyme Immunology Laboratory.

Successful treatment of the patient will exhibit a marked increase in platelet levels after GMA-161 administration and no signs of platelet depletion, with minimal side effects, and preferably no cytokine release syndrome. The maximum tolerated dose can thus be determined in the dose escalation study.

### Example 19

### A Phase I, Open-Label, Two-Center, Single-Dose, Dose-Escalating, Safety, Tolerability, Immunogenicity Study of GMA-161 in Patients with Idiopathic Thrombocytopenic Purpura (ITP)

GMA-161, a humanized antibody to the Fcγ receptor III (FcγRIII, CD16A), is being developed to block the phagocytosis by splenic and hepatic macrophages of antibody-coated platelets. Several clinical studies have been conducted using a murine anti-CD16A mAb 3G8 in patients with refractory ITP. Although a rise in platelet counts was observed in the majority of patients, cytokine release syndrome, transient neutropenia, and human anti-murine antibody (HAMA) responses precluded further clinical development. In addition to being ‘humanized’, GMA-161 has been modified from 3G8 to decrease the ability of its Fc domain to fix complement or bind to receptors on lymphocytes, neutrophils or NK cells.

Patients: The patients will have been diagnosed with ITP for at least 6 months. Preferably the patient will have a platelet count of <50,000/mm³ on 2 determinations at least 6 weeks apart, including 1 determination within 7 days prior to initiating study treatment.

Administration of GAM-161: The patient will receive a single IV infusion of GMA-161 on Day 0 and will be monitored for seven days with collection of blood samples and for safety observation. The patient will receive treatment with GMA-161 dosed at 0.3 mg/kg body weight. GMA-161 is a liquid solution with a protein concentration of 5 mg/mL (25 mg/vial). GMA-161 is in a buffer composed of 5 mM sodium phosphate and 1.7 mM potassium phosphate at pH 7.2, containing 154 mM sodium chloride.

Immunological and Platelet count Assessments: Samples for immunology assessments will be collected various time points specified below: Platelet counts will also be monitored. Blood samples will be collected 2 hrs, 5 hrs, 24 hrs and 48 hrs after administration of GMA-161 and plasma platelet counts will be determined using a particle count and size analyzer Z2™ COULTER COUNTER® (Coulter) equipped with a 70 μm aperture. Data is analyzed by plotting the relative platelet level (the actual platelet count divided by the time 0 platelet count) versus time for each concentration.

Day 0 (pre-infusion), Day 7, Day 21 and Day 28: Anti-platelet assay, antinuclear antibodies (ANA), anti-double stranded DNA antibodies, anticardiolipin antibodies (ACA), lymphocyte subsets (including natural killer cells), and serum immunoglobulin (IgG, IgA, and IgM) concentrations, and serum cytokine assays (e.g., interleukin-6 [IL-6], IL-8, and tumor necrosis factor alpha [TNFα]). If a patient exhibits signs or symptoms of cytokine release syndrome, blood will be collected at 30 minutes post infusion for cytokine assays.

Day 0 (pre-infusion) and Day 7: Serum CD16A and C3 and C4 complement. Pre-infusion anti-GMA-161 antibody serum will be collected at Day 0. Post-infusion anti-GMA-161 antibody assay will start when the blood GMA-161 level no longer interferes with the assay, as determined by the Genzyme Immunology Laboratory.

Successful treatment of the patient will exhibit a marked increase in platelet levels after GMA-161 administration and no signs of platelet depletion, with minimal side effects, and preferably no cytokine release syndrome. The maximum tolerated dose can thus be determined in the dose escalation study.

### Example 20

### A Phase I, Open-Label, Two-Center, Single-Dose, Dose-Escalating, Safety, Tolerability, Immunogenicity Study of GMA-161 in Patients with Idiopathic Thrombocytopenic Purpura (ITP)

GMA-161, a humanized antibody to the Fcγ receptor III (FcγRIII, CD16A), is being developed to block the phagocytosis by splenic and hepatic macrophages of antibody-coated platelets. Several clinical studies have been conducted using a murine anti-CD16A mAb 3G8 in patients with refractory ITP. Although a rise in platelet counts was observed in the majority of patients, cytokine release syndrome, transient neutropenia, and human anti-murine antibody (HAMA) responses precluded further clinical development. In addition to being ‘humanized’, GMA-161 has been modified from 3G8 to decrease the ability of its Fc domain to fix complement or bind to receptors on lymphocytes, neutrophils or NK cells.

Patients: The patients will have been diagnosed with ITP for at least 6 months. Preferably the patient will have a platelet count of <50,000/mm³ on 2 determinations at least 6 weeks apart, including 1 determination within 7 days prior to initiating study treatment.

Administration of GMA-161: The patient will receive a single IV infusion of GMA-161 on Day 0 and will be monitored for seven days with collection of blood samples and for safety observation. The patient will receive treatment with GMA-161 dosed at 1.0 mg/kg body weight. GMA-161 is a liquid solution with a protein concentration of 5 mg/mL (25 mg/vial). GMA-161 is in a buffer composed of 5 mM sodium phosphate and 1.7 mM potassium phosphate at pH 7.2, containing 154 mM sodium chloride.

Immunological and Platelet count Assessments: Samples for immunology assessments will be collected various time points specified below: Platelet counts will also be monitored. Blood samples will be collected 2 hrs, 5 hrs, 24 hrs and 48 hrs after administration of GMA-161 and plasma platelet counts will be determined using a particle count and size analyzer Z2™ COULTER COUNTER® (Coulter) equipped with a 70 μm aperture. Data is analyzed by plotting the relative platelet level (the actual platelet count divided by the time 0 platelet count) versus time for each concentration.

Day 0 (pre-infusion), Day 7, Day 21 and Day 28: Anti-platelet assay, antinuclear antibodies (ANA), anti-double stranded DNA antibodies, anticardiolipin antibodies (ACA), lymphocyte subsets (including natural killer cells), and serum immunoglobulin (IgG, IgA, and IgM) concentrations, and serum cytokine assays (e.g., interleukin-6 [IL-6], IL-8, and tumor necrosis factor alpha [TNFα]). If a patient exhibits signs or symptoms of cytokine release syndrome, blood will be collected at 30 minutes post infusion for cytokine assays.

Day 0 (pre-infusion) and Day 7: Serum CD16A and C3 and C4 complement. Pre-infusion anti-GMA-161 antibody serum will be collected at Day 0. Post-infusion anti-GMA-161 antibody assay will start when the blood GMA-161 level no longer interferes with the assay, as determined by the Genzyme Immunology Laboratory.

Successful treatment of the patient will exhibit a marked increase in platelet levels after GMA-161 administration and no signs of platelet depletion, with minimal side effects, and preferably no cytokine release syndrome. The maximum tolerated dose can thus be determined in the dose escalation study.

### Example 21

### A Phase I, Open-Label, Two-Center, Single-Dose, Dose-Escalating, Safety, Tolerability, Immunogenicity Study of GMA-161 in Patients with Idiopathic Thrombocytopenic Purpura (ITP)

GMA-161, a humanized antibody to the Fcγ receptor III (FcγRIII, CD16A), is being developed to block the phagocytosis by splenic and hepatic macrophages of antibody-coated platelets. Several clinical studies have been conducted using a murine anti-CD16A mAb 3G8 in patients with refractory ITP. Although a rise in platelet counts was observed in the majority of patients, cytokine release syndrome, transient neutropenia, and human anti-murine antibody (HAMA) responses precluded further clinical development. In addition to being ‘humanized’, GMA-161 has been modified from 3G8 to decrease the ability of its Fc domain to fix complement or bind to receptors on lymphocytes, neutrophils or NK cells.

Patients: The patients will have been diagnosed with ITP for at least 6 months. Preferably the patient will have a platelet count of <50,000/mm³ on 2 determinations at least 6 weeks apart, including 1 determination within 7 days prior to initiating study treatment.

Administration of GMA-161: The patient will receive a single IV infusion of GMA-161 on Day 0 and will be monitored for seven days with collection of blood samples and for safety observation. The patient will receive treatment with GMA-161 dosed at 3.0 mg/kg body weight. GMA-161 is a liquid solution with a protein concentration of 5 mg/mL (25 mg/vial). GMA-161 is in a buffer composed of 5 mM sodium phosphate and 1.7 mM potassium phosphate at pH 7.2, containing 154 mM sodium chloride.

Immunological and Platelet count Assessments: Samples for immunology assessments will be collected various time points specified below: Platelet counts will also be monitored. Blood samples will be collected 2 hrs, 5 hrs, 24 hrs and 48 hrs after administration of GMA-161 and plasma platelet counts will be determined using a particle count and size analyzer Z2™ COULTER COUNTER® (Coulter) equipped with a 70 μm aperture. Data is analyzed by plotting the relative platelet level (the actual platelet count divided by the time 0 platelet count) versus time for each concentration.

Day 0 (pre-infusion), Day 7, Day 21 and Day 28: Anti-platelet assay, antinuclear antibodies (ANA), anti-double stranded DNA antibodies, anticardiolipin antibodies (ACA), lymphocyte subsets (including natural killer cells), and serum immunoglobulin (IgG, IgA, and IgM) concentrations, and serum cytokine assays (e.g., interleukin-6 [IL-6], IL-8, and tumor necrosis factor alpha [TNFα]). If a patient exhibits signs or symptoms of cytokine release syndrome, blood will be collected at 30 minutes post infusion for cytokine assays.

Day 0 (pre-infusion) and Day 7: Serum CD16A and C3 and C4 complement. Pre-infusion anti-GMA-161 antibody serum will be collected at Day 0. Post-infusion anti-GMA-161 antibody assay will start when the blood GMA-161 level no longer interferes with the assay, as determined by the Genzyme Immunology Laboratory.

Successful treatment of the patient will exhibit a marked increase in platelet levels after GMA-161 administration and no signs of platelet depletion, with minimal side effects, and preferably no cytokine release syndrome. The maximum tolerated dose can thus be determined in the dose escalation study.

It is understood that the examples and embodiments described herein are for illustrative purposes only and that various modifications or changes in light thereof will be suggested to persons skilled in the art and are to be included within the spirit and purview of this application and scope of the appended claims. All publications (including sequence accession numbers and corresponding annotations), patents and patent applications cited herein are hereby incorporated by reference in their entirety for all purposes to the same extent as if each individual publication, patent or patent application were specifically and individually indicated to be so incorporated by reference.

What is claimed is:

1. A method of treating a deleterious immune response in a mammal without inducing severe neutropenia in the mammal, optionally without inducing moderate neutropenia in the mammal, wherein the method comprises administering to the mammal a humanized anti-CD16A antibody that has reduced effector function and comprises all six complementarity determining regions of mouse antibody 3G8, and wherein the antibody is administered at a dose of approximately 0.1-30.0 mg/kg of the mammal's body weight.

2. A method of treating a deleterious immune response in a mammal without inducing severe neutropenia in the mammal, optionally without inducing moderate neutropenia in the mammal, wherein the method comprises administering to the mammal an anti-CD16A antibody comprising a VH domain comprising complementarity determining regions (CDRs) derived from the mouse 3G8 antibody heavy chain and a VL domain comprising CDRs derived from the mouse 3G8 antibody light chain, wherein at least one of said CDRs differs from the corresponding mouse CDR at at least one position selected from the group consisting of, in the VH domain, Val at position 34 in CDR1, Leu at position 50 in CDR2, Phe at position 52 in CDR2, Asn at position 54 in CDR2, Ser at position 60 in CDR2, Ser at position 62 in CDR2, Tyr at position 99 in CDR3, Asp at position 101 of CDR3, and, in the VL domain, Arg at position 24 in CDR1, Ser at position 25 in CDR1, Tyr at position 32 in CDR1, Leu at position 33 in CDR1, Ala at position 34 in CDR1, Asp, Trp or Ser at position 50 in CDR2, Ala at position 51 in CDR2, Ser at position 53 in CDR2, Ala or Gln at position 55 in CDR2, Thr at position 56 in CDR2, Tyr at position 92 in CDR3, Ser at position 93 in CDR3, and Thr at position 94 in CDR3, wherein the anti-CD16A antibody has an Fc region that has reduced effector function, and wherein the antibody is administered at a dose of approximately 0.1-30.0 mg/kg of the mammal's body weight.

3. The method of claim 1, wherein the deleterious immune response is an inflammatory response caused by an autoimmune disease.

4. The method of claim 3, wherein treating a deleterious immune response comprises protecting against antibody-mediated platelet depletion.

5. A method of reducing a deleterious immune response in a mammal in need of such reduction, comprising administering to the mammal a CD16A binding protein comprising an Fc region derived from a human IgG heavy chain, wherein the Fc region has reduced effector function or is modified to reduce binding to an Fc receptor, and wherein the CD16A binding protein is administered at a dose of approximately 0.1-30.0 mg/kg of the mammal's body weight.

6. The method of claim 5 wherein the CD16A binding protein is a humanized monoclonal antibody, and wherein the antibody is administered at a dose of approximately 0.1-30.0 mg/kg of the mammal's body weight.

7. The method of claim 6 wherein an amino acid residue at position 297 of the Fc region is not glycosylated or is not asparagine.

8. The method of claim 6 wherein the antibody is a humanized form of the 3G8 antibody or inhibits CD16A binding by the 3G8 antibody.

9. The method of claim 6 wherein the antibody comprises a VH domain comprising complementarity determining regions (CDRs) derived from the mouse 3G8 antibody heavy chain and a VL domain comprising CDRs derived from the mouse 3G8 antibody light chain, wherein at least one of said CDRs differs from the corresponding mouse CDR at at least one position selected from the group consisting of, in the VH domain, Val at position 34 in CDR1, Leu at position 50 in CDR2, Phe at position 52 in CDR2, Asn at position 54 in CDR2, Ser at position 60 in CDR2, Ser at position 62 in CDR2, Tyr at position 99 in CDR3, Asp at position 101 of CDR3, and, in the VL domain, Arg at position 24 in CDR1, Ser at position 25 in CDR1, Tyr at position 32 in CDR1, Leu at position 33 in CDR1, Ala at position 34 in CDR1, Asp, Tip or Ser at position 50 in CDR2, Ala at position 51 in CDR2, Ser at position 53 in CDR2, Ala or Gln at position 55 in CDR2, Thr at position 56 in CDR2, Tyr at position 92 in CDR3, Ser at position 93 in CDR3, and Thr at position 94 in CDR3.

10. The method of claim 6 wherein the deleterious immune response is idiopathic thrombocytopenic purpura, autoimmune hemolytic anemia, or an inflammatory response caused by an autoimmune disease.

11. A method of reducing a deleterious immune response in a mammal, comprising administering to the mammal an effective amount of an anti-CD16A antibody which specifically binds CD16A via an interaction with its VL and/or VH domains, and wherein the anti-CD16A antibody comprises an Fc region with reduced effector function or one or more amino acid modifications in the Fc region so that binding to an Fc receptor is reduced, and wherein the anti-CD16A antibody is administered at a dose of approximately 0.1-30.0 mg/kg of the mammal's body weight.

12. The method of claim 11 wherein the anti-CD16A antibody comprises a VH domain comprising complementarity determining regions (CDRs) derived from the mouse 3G8 antibody heavy chain and a VL domain comprising CDRs derived from the mouse 3G8 antibody light chain, wherein the antibody comprises an Fc region with reduced effector function.

13. The method of claim 11 wherein the anti-CD16A antibody comprises a VH domain comprising complementarity determining regions (CDRs) derived from the mouse 3G8 antibody heavy chain and a VL domain comprising CDRs derived from the mouse 3G8 antibody light chain, wherein the antibody comprises one or more amino acid modifications in the Fc region so that binding to an Fc receptor is reduced.

14. The method of claim 11 or 13, wherein the anti-CD16A antibody comprises an Fc region derived from human IgG1, wherein the amino acid corresponding to position 297 of the CH2 domain of the Fc region is not an asparagine or is not glycosylated.

15. The method of claim 1 or 12, wherein the anti-CD16A antibody is administered at a dose of approximately 0.1-20.0, 0.1-10.0, or 0.1-5.0 mg/kg of the mammal's body weight.

16. The method of claim 1 or 12, wherein the anti-CD16A antibody is administered at a dose of approximately 1.0-30.0, 1.0-20.0, 1.0-10.0 or 5.0-20.0 mg/kg of the mammal's body weight.

17. The method of claim 1 or 12, wherein the anti-CD16A antibody is administered at a dose of approximately 0.1, 0.3, 1.0, or 3.0 mg/kg of the mammal's body weight.

18. The method of claim 1 or 12, wherein the anti-CD16A antibody is administered at least once a day.

19. The method of claim 1 or 12, wherein the anti-CD16A antibody is administered once a week, twice a week, once every two weeks, once a month, once every six weeks, once every two months, twice a year or once a year.

20. The method of claim 1 or 12, wherein the anti-CD16A antibody is administered to the mammal topically, orally, intravenously, intradermally, or subcutaneously.

21. The method of claim 1 or 12, wherein the anti-CD16A antibody is administered to the mammal intravenously over at least 15 minutes.
